(19) United States
(12) Patent Application Publication
(10) Pub. No.: US 2022/0241294 A1
(43) Pub. Date: Aug. 4, 2022

(54) BISFLUOROALKYL-1,4-BENZODIAZEPINONE COMPOUNDS FOR TREATING NOTCH-ACTIVATED BREAST CANCER

(71) Applicant: Ayala Pharmaceuticals Inc., Wilmington, DE (US)

(72) Inventor: Matti DAVIS, Modiin (IL)

(73) Assignee: Ayala Pharmaceuticals Inc., Wilmington, DE (US)

(21) Appl. No.: 17/611,185

(22) PCT Filed: May 14, 2020
(86) PCT No.: PCT/US2020/032786

§ 371 Date: Nov. 15, 2021

Related U.S. Application Data
(60) Provisional application No. 62/847,969, filed on May 15, 2019.

(51) Int. Cl. A61K 31/5513; A61K 45/06; A61K 31/573; A61P 35/00

(52) U.S. Cl. CPC A61K 31/5513; A61P 35/00; A61K 31/573; A61K 45/06

(57) ABSTRACT

The present invention provides methods of reducing tumor size, suppressing or inhibiting tumor growth, or prolonging progression-free survival or overall survival in subjects having Notch-activated breast cancer by administering compositions comprising bisfluoroalkyl-1,4-benzodiazepinone compounds, including compounds of Formula (III): or prodrugs thereof, alone or in combination with a composition comprising a cytotoxic agent. Notch-activated breast cancer may be determined by a) Notch-activating genetic alterations in one or more Notch genes, b) overexpression of one or more Notch-regulated genes, c) overexpression of one or more Notch proteins or Notch-regulated proteins, or a combination thereof.

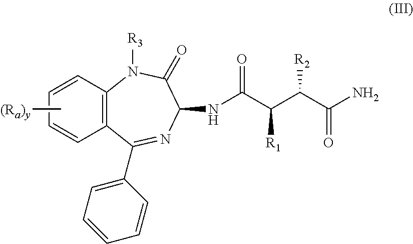

### FIELD OF THE INVENTION

The present invention provides methods of reducing tumor size, suppressing or inhibiting tumor growth, or prolonging progression-free survival or overall survival in subjects having Notch-activated breast cancer by administering compositions comprising bisfluoroalkyl-1,4-benzodiazepinone compounds, including compounds of Formula (III):

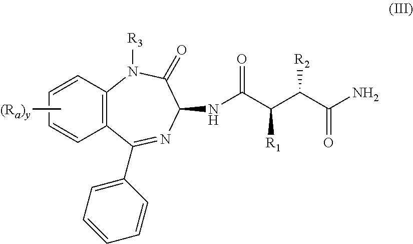

or prodrugs thereof, alone or in combination with a composition comprising a cytotoxic agent. Notch-activated breast cancer may be determined by a) Notch-activating genetic alterations in one or more Notch genes, b) overexpression of one or more Notch-regulated genes, c) overexpression of one or more Notch proteins or Notch-regulated proteins, or a combination thereof.

### BACKGROUND OF THE INVENTION

The Notch pathway is activated during normal breast development and has been implicated as a key driver in breast cancer. Within breast cancer, triple-negative breast cancer (TNBC) is associated with a poor prognosis and lack of available targeted therapies. Expression of estrogen receptor (ER), progesterone receptor, and HER2 is lacking in TNBC cells. TNBC accounts for 15% to 20% of the cases of invasive breast cancer. These tumors have a more aggressive phenotype and a poorer prognosis due to the high propensity for metastatic progression and absence of specific targeted treatments. Patients with TNBC do not benefit from hormonal or trastuzumab-based targeted therapies because of the loss of target receptors. Although these patients respond to chemotherapeutic agents such as taxanes and anthracyclines better than other subtypes of breast cancer, prognosis remains poor. TNBC is usually aggressive, with higher undifferentiated cell morphology or frequent nodal metastasis, and usually develops at a higher rate in young patients. Patients with TNBC tend to experience an increased likelihood of distant metastasis and early recurrence within 2 or 3 years after treatment, compared with patients with other subtypes of breast cancer; patients with TNBC also tend to have shorter survival. Notch genetic alterations are potential tumor drivers and have been identified in ˜10% of TNBC. Identifying new therapeutic strategies for triple-negative breast cancer (TNBC) would satisfy an unmet need.

### SUMMARY OF THE INVENTION

The present invention provides a method of reducing tumor size, suppressing tumor growth, or inhibiting tumor growth having breast cancer characterized by an activated Notch pathway, comprising the step of administering to said subject a composition comprising one or more compounds represented by the structure of Formula (III):

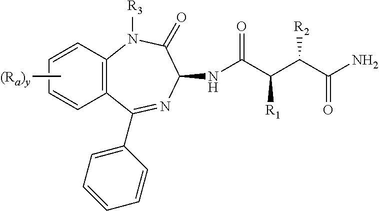

or prodrugs or salts thereof; wherein:
R₁ is —CH₂CF₃ or —CH₂CH₂CF₃;
R₂ is —CH₂CF₃, —CH₂CH₂CF₃, or —CH₂CH₂CH₂CF₃;
R₃ is H or —CH₃;
each Rₐ is independently F, Cl, —CN, —OCH₃, and/or —NHCH₂CH₂OCH₃; and
y is zero, 1, or 2.

The present invention also provides a method of reducing tumor size, suppressing tumor growth, or inhibiting tumor growth in a subject having breast cancer characterized by an activated Notch pathway, comprising the step of administering to said subject a first composition comprising a cytotoxic agent and a second composition comprising one or more compounds represented by the structure of Formula (III):

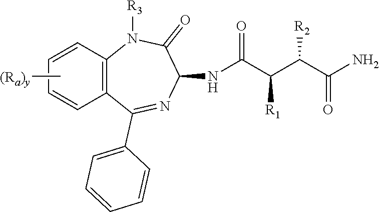

or prodrugs or salts thereof; wherein:
R₁ is —CH₂CF₃ or —CH₂CH₂CF₃;
R₂ is —CH₂CF₃, —CH₂CH₂CF₃, or —CH₂CH₂CH₂CF₃;
R₃ is H or —CH₃;
each Rₐ is independently F, Cl, —CN, —OCH₃, and/or —NHCH₂CH₂OCH₃; and
y is zero, 1, or 2.

### BRIEF DESCRIPTION OF THE DRAWINGS

The subject matter regarded as the invention is particularly pointed out and distinctly claimed in the concluding portion of the specification. The invention, however, both as to organization and method of operation, together with objects, features, and advantages thereof, may best be understood by reference to the following detailed description when read with the accompanying drawings in which:

FIGS. 1A-J: Correlation between Notch activation signature and Compound (1) efficacy. Tumor volume as a function of days post treatment with vehicle (circles) or Compound (1) (squares) in Patient Derived Xenograft (PDX) models which harbor a Notch-on gene expression signature, CTG-1374 (FIG. 1A), CTG-1408 (FIG. 1B), CTG-2010 (FIG. 1C), CTG-1340 (FIG. 1D), and CTG-2488 (FIG. 1E). Tumor volume as a function of days post treatment with vehicle (circles) or Compound (1) (squares) in PDX models which do NOT harbor a Notch activating signature, CTG-1646 (FIG. 1F), CTG-1167 (FIG. 1G), CTG-1941 (FIG. 1H), CTG-0017 (FIG. 1I) and CTG-1520 (FIG. 1J). ** Data shown as mean±SEM, n=5 animals/group except dotted line where n=4.

FIGS. 2A-B: Heat map showing Notch-on gene expression signature and Notch gene alterations in Breast Cancer Patient-Derived Xenograft (PDX) models. The differential expression of 21 Notch-regulated genes (HEY1, NOTCH1, HEYL, NOTCH2, OLFM4, MYC, CDK6, HEY2, KIT, NRARP, MVP, HES6, CDKN2D, NOTCH4, NOTCH3, HES4, HES5, CCND1, HES1, CDKN1B, HES2) and identified Notch genetic alterations (NRR/PEST, Loss of Function (LOF), wild-type, gene fusion or internal deletion, or variant of unknown significance (VUS)) in 65 Patient-Derived Xenograft (PDX) breast cancer models from Champions' TumorGraft® database. The activated Notch-on cluster is shown on the left and is enriched with Notch genetic alterations such as fusions, internal deletions and NRR/PEST mutations. FIG. 2B is an enlargement of the activated Notch cluster from FIG. 2A.

FIG. 3: Effect of prior treatment with Compound (1) alone and in combination with Eribulin on tumor re-growth after treatment withdrawal. A Notch-activated TNBC PDX tumor (CTG-1374) was implanted subcutaneously (flank) into Nude Mice. Treatment with either vehicle, Compound (1) (3 mg/kg PO 4on/3off)), Eribulin (0.5 mg/kg IV QW) or the combination of Compound (1) & Eribulin was initiated when the average tumor volume was ˜200 mm³. On Day 40, the initial treatments were stopped. Tumor volume is expressed in mm³. Treatment was discontinued after 4 weeks and tumors were followed for potential regrowth.

FIGS. 4A-B: Effect of second-round treatment with Compound (1) in combination with Eribulin on tumor growth. Notch-activated TNBC PDX tumors (CTG-1374) were implanted into mice. Treatment with Eribulin (0.5 mg/kg IV QW) (FIG. 4A) or the combination of Compound (1) & Eribulin (FIG. 4B) was initiated when the average tumor volume was ˜200 mm³. Treatment was halted on day 28, and tumors began to regrow. Once tumors reached an average tumor volume of ˜650 mm³ (FIGS. 4A-4B), the mice were re-randomized into 2 treatment arms with Eribulin alone (0.25 mg/kg IV QW), or Compound (1) (3 mg/kg PO 4on/3off) in combination with Eribulin.

It will be appreciated that for simplicity and clarity of illustration, elements shown in the figures have not necessarily been drawn to scale. For example, the dimensions of some of the elements may be exaggerated relative to other elements for clarity. Further, where considered appropriate, reference numerals may be repeated among the figures to indicate corresponding or analogous elements.

### DETAILED DESCRIPTION OF THE PRESENT INVENTION

In the following detailed description, numerous specific details are set forth in order to provide a thorough understanding of the invention. However, it will be understood by those skilled in the art that the present invention may be practiced without these specific details. In other instances, well-known methods, procedures, and components have not been described in detail so as not to obscure the present invention.

In one embodiment, compositions of the present invention or for use in the methods of the present invention comprise one or more gamma secretase inhibitors, one or more Notch inhibitors, or a combination thereof. In one embodiment, the gamma secretase inhibitor comprises a bisfluoroalkyl-1,4-benzodiazepinone compound.

Bisfluoroalkyl-1,4-benzodiazepinone Compounds

In one embodiment, the present invention provides compositions comprising compounds represented by the structure of Formula (I):

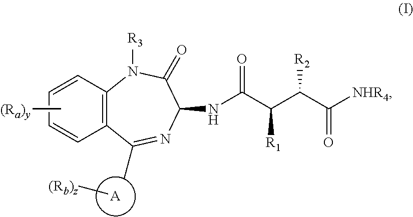

and/or at least one salt thereof, wherein:
R₁ is —CH₂CF₃ or —CH₂CH₂CF₃;
R₂ is —CH₂CF₃, —CH₂CH₂CF₃, or —CH₂CH₂CH₂CF₃;
R₃ is H, —CH₃ or Rx;
R₄ is H or R_{y};
Rₓ
is: —CH₂OC(O)CH(CH₃)NH₂, —CH₂OC(O)CH(NH₂)CH(CH₃)₂, —CH₂OC(O)CH((CH(CH₃)₂)NHC(O)CH(NH₂)CH(CH₃)₂,

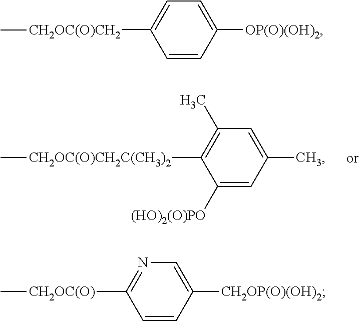

R_{y} is: —SCH₂CH(NH₂)C(O)OH, —SCH₂CH(NH₂)C(O)OH₃,
or —SCH₂CH(NH₂)C(O)OC(CH₃)₃;
Ring A is phenyl or pyridinyl;
each Rₐ is independently F, Cl, —CN, —OCH₃, C₁₋₃ alkyl, —CH₂OH, —CF₃, cyclopropyl, —OCH₃, —O(cyclopropyl) and/or —NHCH₂CH₂OCH₃;
each R_{b} is independently F, Cl, —CH₃, —CH₂OH, —CF₃, cyclopropyl, and/or —OCH₃;
y is zero, 1 or 2; and
z is zero, 1, or 2.

In one embodiment, the present invention provides compositions comprising compounds as described herein formulated at a dose of 4 mg. In one embodiment, the present invention provides compositions comprising compounds as described herein formulated for intravenous administration.

In one embodiment, the present invention provides compositions comprising compounds represented by the structure of Formula (II):

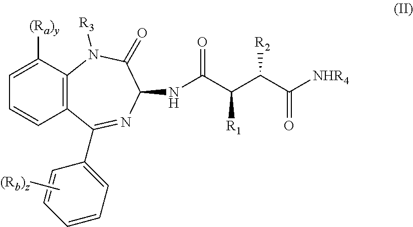

wherein R₃ is H or —CH₃; and y is zero or 1.

In one embodiment, the present invention provides compositions comprising compounds of Formula (III):

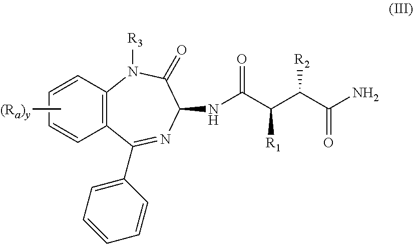

or prodrugs or salts thereof; wherein:
R₁ is —CH₂CF₃ or —CH₂CH₂CF₃;
R₂ is —CH₂CF₃, —CH₂CH₂CF₃, or —CH₂CH₂CH₂CF₃;
R₃ is H or —CH₃;
each Rₐ is independently F, Cl, —CN, —OCH₃, and/or —NHCH₂CH₂OCH₃; and
y is zero, 1, or 2.

In one embodiment, R₁ is —CH₂CF₃ or —CH₂CH₂CF₃ and R₂ is —CH₂CF₃ or —CH₂CH₂CF₃. In another embodiment, R₁ is —CH₂CH₂CF₃ and R₂ is —CH₂CH₂CF₃. In one embodiment, y is 1 or 2. In another embodiment, y is zero or 1. In one embodiment, y is zero.

In one embodiment, the compound of Formula (III) comprises: (2R,3S)-N-((3S)-1-methyl-2-oxo-5-phenyl-2,3-dihydro-1H-1,4-benzodiazepin-3-yl)-2,3-bis(3,3,3-trifluoropropyl)succinamide (1)

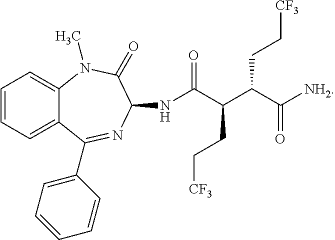 [SMILES: CN1C(=O)[C@@H](NC(=O)[C@H](CCC(F)(F)F)[C@H](CCC(F)(F)F)C(N)=O)N=C(c2ccccc2)c2ccccc21]

In another embodiment, the compound of Formula (III) comprises: (2R,3S)-N-((3S)-2-oxo-5-phenyl-2,3-dihydro-1H-1,4-benzodiazepin-3-yl)-2,3-bis(3,3,3-trifluoropropyl)succinamide (2)

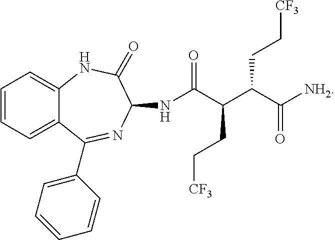 [SMILES: NC(=O)[C@@H](CCC(F)(F)F)[C@@H](CCC(F)(F)F)C(=O)N[C@H]1N=C(c2ccccc2)c2ccccc2NC1=O]

In another embodiment, the compound of Formula (III) comprises: (2R,3S)-N-((3S)-1-methyl-2-oxo-5-phenyl-2,3-dihydro-1H-1,4-benzodiazepin-3-yl)-2-(2,2,2-trifluoroethyl)-3-(3,3,3-trifluoropropyl)succinamide (3);

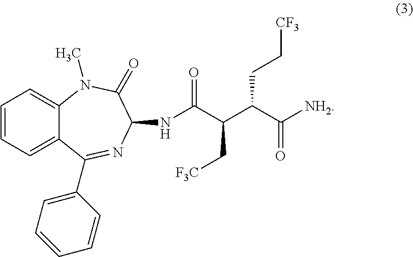 [SMILES: CN1C(=O)[C@@H](NC(=O)[C@H](CC(F)(F)F)[C@H](CCC(F)(F)F)C(N)=O)N=C(c2ccccc2)c2ccccc21]

In another embodiment, the compound of Formula (III) comprises: (2R,3S)-N-((3S)-1-methyl-2-oxo-5-phenyl-2,3-dihydro-1H-1,4-benzodiazepin-3-yl)-3-(2,2,2-trifluoroethyl)-2-(3,3,3-trifluoropropyl)succinamide (4);

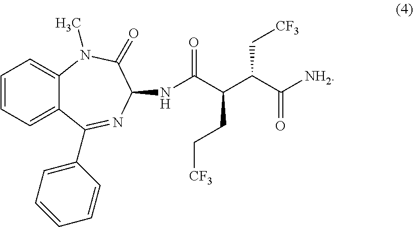 [SMILES: CN1C(=O)[C@@H](NC(=O)[C@H](CCC(F)(F)F)[C@H](CC(F)(F)F)C(N)=O)N=C(c2ccccc2)c2ccccc21]

In another embodiment, the compound of Formula (III) comprises: (2R,3S)-N-((3S)-1-(²H3)methyl-2-oxo-5-phenyl-2,3-dihydro-1H-1,4-benzodiazepin-3-yl)-2,3-bis(3,3,3-trifluoropropyl)succinamide (5);

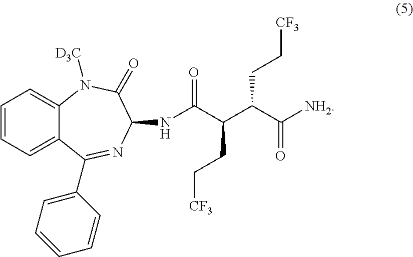 [SMILES: [2H]C([2H])([2H])N1C(=O)[C@@H](NC(=O)[C@H](CCC(F)(F)F)[C@H](CCC(F)(F)F)C(N)=O)N=C(c2ccccc2)c2ccccc21]

In another embodiment, the compound of Formula (III) comprises a compound of Formula (VI):

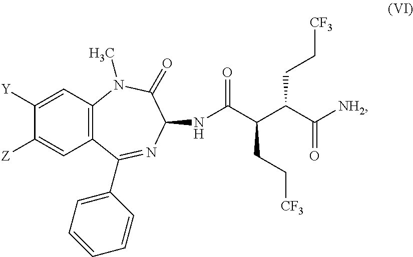

which in one embodiment, comprises (2R,3S)-N-((3S)-7-chloro-1-methyl-2-oxo-5-phenyl-2,3-dihydro-1H-1,4-benzodiazepin-3-yl)-2,3-bis(3,3,3-trifluoropropyl)succinamide (6), i.e. Y═H and Z═Cl; (2R,3S)-N-((3S)-8-methoxy-1-methyl-2-oxo-5-phenyl-2,3-dihydro-1H-1,4-benzodiazepin-3-yl)-2,3-bis(3,3,3-trifluoropropyl)succinamide (7), i.e. Y═OCH₃ and Z═H; (2R,3S)-N-((3S)-8-fluoro-1-methyl-2-oxo-5-phenyl-2,3-dihydro-1H-1,4-benzodiazepin-3-yl)-2,3-bis(3,3,3-trifluoropropyl)succinamide (8), i.e. Y═F and Z═H; (2R,3S)-N-((3S)-7-methoxy-1-methyl-2-oxo-5-phenyl-2,3-dihydro-1H-1,4-benzodiazepin-3-yl)-2,3-bis(3,3,3-trifluoropropyl)succinamide (9), Y═H and Z═OCH₃; (2R,3S)-N-((3S)-7-fluoro-1-methyl-2-oxo-5-phenyl-2,3-dihydro-1H-1,4-benzodiazepin-3-yl)-2,3-bis(3,3,3-trifluoropropyl)succinamide (10), i.e. Y═H and Z═F; or (2R,3S)-N-((3S)-8-chloro-1-methyl-2-oxo-5-phenyl-2,3-dihydro-1H-1,4-benzodiazepin-3-yl)-2,3-bis(3,3,3-trifluoropropyl)succinamide (11), i.e. Y═Cl and Z═H.

In another embodiment, the compound of Formula (III) comprises a compound of Formula (VII):

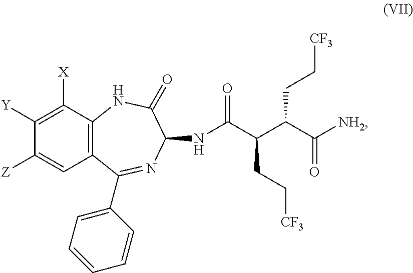

which in one embodiment, comprises (2R,3S)-N-((3S)-9-methoxy-2-oxo-5-phenyl-2,3-dihydro-1H-1,4-benzodiazepin-3-yl)-2,3-bis(3,3,3-trifluoropropyl)succinamide (12), i.e. X═OCH₃, Y═H and Z═H; (2R,3S)-N-((3S)-8-methoxy-2-oxo-5-phenyl-2,3-dihydro-1H-1,4-benzodiazepin-3-yl)-2,3-bis(3,3,3-trifluoropropyl)succinamide (13), i.e. X═H, Y═OCH₃ and Z═H; (2R,3S)-N-((3S)-7-methoxy-2-oxo-5-phenyl-2,3-dihydro-1H-1,4-benzodiazepin-3-yl)-2,3-bis(3,3,3-trifluoropropyl)succinamide (14), i.e. X═H, Y═H and Z═OCH₃; (2R,3S)-N-((3S)-8-cyano-9-methoxy-2-oxo-5-phenyl-2,3-dihydro-1H-1,4-benzodiazepin-3-yl)-2,3-bis(3,3,3-trifluoropropyl)succinamide (15), i.e. X═OCH₃, Y═CN and Z═H; (2R,3S)-N-((3S)-8,9-dichloro-2-oxo-5-phenyl-2,3-dihydro-1H-1,4-benzodiazepin-3-yl)-2,3-bis(3,3,3-trifluoropropyl)succinamide (16), i.e. X═Cl, Y═Cl and Z═H; (2R,3S)-N-((3S)-9-fluoro-2-oxo-5-phenyl-2,3-dihydro-1H-1,4-benzodiazepin-3-yl)-2,3-bis(3,3,3-trifluoropropyl)succinamide (17), i.e. X═F, Y═H and Z═H; or (2R,3S)-N-((3S)-9-chloro-2-oxo-5-phenyl-2,3-dihydro-1H-1,4-benzodiazepin-3-yl)-2,3-bis(3,3,3-trifluoropropyl)succinamide (18), i.e. X═Cl, Y═H and Z═H.

In another embodiment, the compound of Formula (III) comprises: (2R,3S)-N-((3S)-2-oxo-5-phenyl-2,3-dihydro-1H-1,4-benzodiazepin-3-yl)-3-(4,4,4-trifluorobutyl)-2-(3,3,3-trifluoropropyl)succinamide (19);

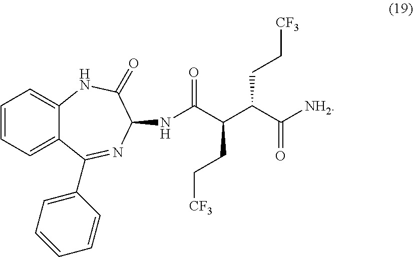 [SMILES: NC(=O)[C@@H](CCC(F)(F)F)[C@@H](CCC(F)(F)F)C(=O)N[C@H]1N=C(c2ccccc2)c2ccccc2NC1=O]

In another embodiment, the compound of Formula (III) comprises: (2R,3S)-N-((3S)-8-methoxy-2-oxo-5-phenyl-2,3-dihydro-1H-1,4-benzodiazepin-3-yl)-3-(4,4,4-trifluorobutyl)-2-(3,3,3-trifluoropropyl)succinamide (20)

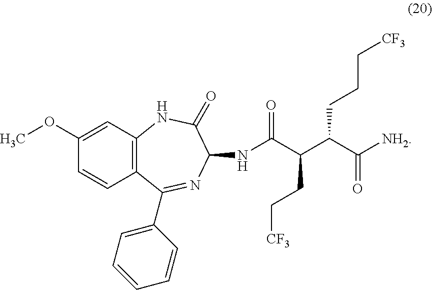 [SMILES: COc1ccc2c(c1)NC(=O)[C@@H](NC(=O)[C@H](CCC(F)(F)F)[C@H](CCCC(F)(F)F)C(N)=O)N=C2c1ccccc1]

In another embodiment, the compound of Formula (III) comprises: (2R,3S)-N-((3S)-9-((2-methoxyethyl)amino)-2-oxo-5-phenyl-2,3-dihydro-1H-1,4-benzodiazepin-3-yl)-2,3-bis(3,3,3-trifluoropropyl)succinamide (21)

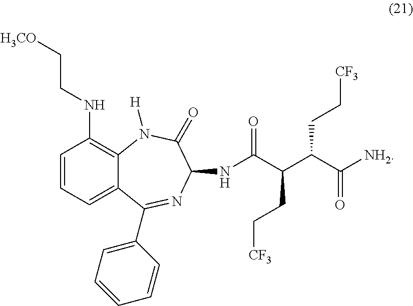 [SMILES: COCCNc1cccc2c1NC(=O)[C@@H](NC(=O)[C@H](CCC(F)(F)F)[C@H](CCC(F)(F)F)C(N)=O)N=C2c1ccccc1]

In another embodiment, the present invention provides compositions comprising compounds represented by the structure of Formula (I):

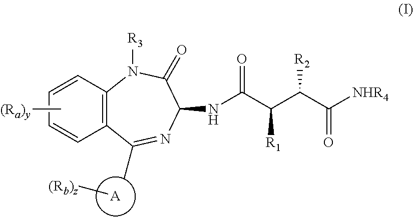

and/or at least one salt thereof, wherein:
R₁ is —CH₂CF₃;
R₂ is —CH₂CH₂CF₃, or —CH₂CH₂CH₂CF₃;
R₃ is H, —CH₃ or Rₓ;
R₄ is H or R_{y};
Rₓ
is: —CH₂OC(O)CH(CH₃)NH₂, —CH₂OC(O)CH(NH₂)CH(CH₃)₂, —CH₂OC(O)CH((CH(CH₃)₂)NHC(O)CH(NH₂)CH(CH₃)₂,

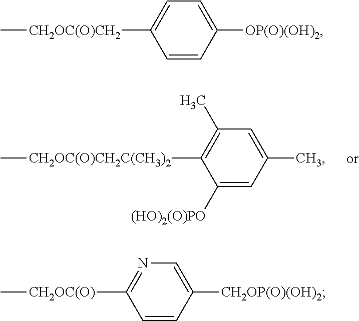

R_{y} is: —SCH₂CH(NH₂)C(O)OH, —SCH₂CH(NH₂)C(O)OH₃,
or —SCH₂CH(NH₂)C(O)OC(CH₃)₃;
Ring A is phenyl or pyridinyl;
each Rₐ is independently Cl, C₁₋₃ alkyl, —CH₂OH, —CF₃, cyclopropyl, —OCH₃,
and/or —O(cyclopropyl);
each R_{b} is independently F, Cl, —CH₃, —CH₂OH, —CF₃, cyclopropyl, and/or —OCH₃;
y is zero, 1 or 2; and
is 1 or 2.

In another embodiment, Ring A is phenyl; and R₃ is H. In another embodiment, R₂ is —CH₂CH₂CF₃; and Ring A is phenyl. In another embodiment, R₂ is —CH₂CH₂CF₃; Ring A is phenyl; Rₐ is C₁₋₃ alkyl or —CH₂OH; each R_{b} is independently F and/or Cl; and y is 1.

In another embodiment, the present invention provides compositions comprising compounds represented by the structure of Formula (IV):

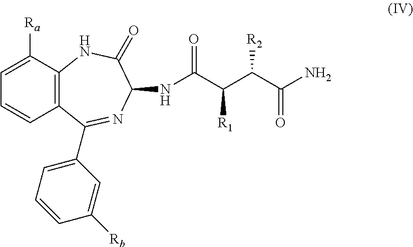

In another embodiment, the present invention provides compositions comprising compounds represented by the structure of Formula (V):

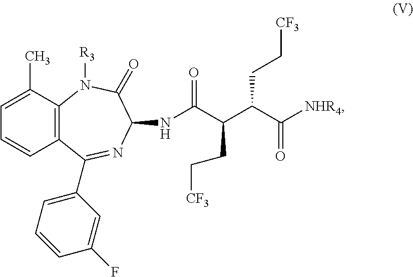

wherein R₃ is H or Rₓ.

In another embodiment, the present invention provides compositions comprising (2R,3S)-N-((3S)-5-(3-fluorophenyl)-9-methyl-2-oxo-2,3-dihydro-1H-1,4-benzodiazepin-3-yl)-2,3-bis(3,3,3-trifluoropropyl)succinamide (22); (2R,3S)-N-((3S)-5-(3-chlorophenyl)-9-ethyl-2-oxo-2,3-dihydro-1H-1,4-benzodiazepin-3-yl)-2,3-bis(3,3,3-trifluoropropyl)succinamide (23); (2R,3S)-N-((3S)-5-(3-chlorophenyl)-9-isopropyl-2-oxo-2,3-dihydro-1H-1,4-benzodiazepin-3-yl)-2,3-bis(3,3,3-trifluoropropyl)succinamide (24); (2R,3S)-N-(9-chloro-5-(3,4-dimethylphenyl)-2-oxo-2,3-dihydro-1H-1,4-benzodiazepin-3-yl)-3-(4,4,4-trifluorobutyl)-2-(3,3,3-trifluoropropyl)succinamide (25); (2R,3S)-N-(9-chloro-5-(3,5-dimethylphenyl)-2-oxo-2,3-dihydro-1H-1,4-benzodiazepin-3-yl)-3-(4,4,4-trifluorobutyl)-2-(3,3,3-trifluoropropyl)succinamide (26); (2R,3S)-N-((3S)-9-ethyl-5-(3-methylphenyl)-2-oxo-2,3-dihydro-1H-1,4-benzodiazepin-3-yl)-2,3-bis(3,3,3-trifluoropropyl)succinamide (27); (2R,3S)-N-((3S)-5-(3-chlorophenyl)-9-methyl-2-oxo-2,3-dihydro-1H-1,4-benzodiazepin-3-yl)-2,3-bis(3,3,3-trifluoropropyl)succinamide (28); (2R,3S)-N-((3S)-5-(3-chlorophenyl)-9-methyl-2-oxo-2,3-dihydro-1H-1,4-benzodiazepin-3-yl)-3-(4,4,4-trifluorobutyl)-2-(3,3,3-trifluoropropyl)succinamide (29); (2R,3S)-N-((3S)-5-(3-methylphenyl)-2-oxo-9-(trifluoromethyl)-2,3-dihydro-1H-1,4-benzodiazepin-3-yl)-2,3-bis(3,3,3-trifluoropropyl)succinamide (30); (2R,3S)-N-((3S)-9-chloro-5-(3,5-dimethylphenyl)-2-oxo-2,3-dihydro-1H-1,4-benzodiazepin-3-yl)-2,3-bis(3,3,3-trifluoropropyl)succinamide (31); (2R,3S)-N-((3S)-5-(3-methylphenyl)-2-oxo-9-(trifluoromethyl)-2,3-dihydro-1H-1,4-benzodiazepin-3-yl)-3-(4,4,4-trifluorobutyl)-2-(3,3,3-trifluoropropyl)succinamide (32); (2R,3S)-N-((3S)-9-isopropyl-5-(3-methylphenyl)-2-oxo-2,3-dihydro-1H-1,4-benzodiazepin-3-yl)-2,3-bis(3,3,3-trifluoropropyl)succinamide (33); (2R,3S)-N-((3S)-9-(cyclopropyloxy)-5-(3-methylphenyl)-2-oxo-2,3-dihydro-1H-1,4-benzodiazepin-3-yl)-3-(4,4,4-trifluorobutyl)-2-(3,3,3-trifluoropropyl)succinamide (34); (2R,3S)-N-((3S)-9-(cyclopropyloxy)-5-(3-methylphenyl)-2-oxo-2,3-dihydro-1H-1,4-benzodiazepin-3-yl)-2,3-bis(3,3,3-trifluoropropyl)succinamide (35); (2R,3S)-N-((3S)-9-chloro-5-(3-methylphenyl)-2-oxo-2,3-dihydro-1H-1,4-benzodiazepin-3-yl)-3-(4,4,4-trifluorobutyl)-2-(3,3,3-trifluoropropyl) succinamide (36); (2R,3S)-N-((3S)-9-methyl-2-oxo-5-(3-(trifluoromethyl)phenyl)-2,3-dihydro-1H-1,4-benzodiazepin-3-yl)-3-(4,4,4-trifluorobutyl)-2-(3,3,3-trifluoropropyl) succinamide (37); (2R,3S)-N-((3S)-9-methyl-2-oxo-5-(3-(trifluoromethyl) phenyl)-2,3-dihydro-1H-1,4-benzodiazepin-3-yl)-2,3-bis(3,3,3-trifluoropropyl) succinamide (38); (2R,3S)-N-((3S)-9-chloro-5-(2-methylphenyl)-2-oxo-2,3-dihydro-1H-1,4-benzodiazepin-3-yl)-2,3-bis(3,3,3-trifluoropropyl)succinamide (39); (2R,3S)-N-((3S)-5-(4-fluorophenyl)-9-methyl-2-oxo-2,3-dihydro-1H-1,4-benzodiazepin-3-yl)-2,3-bis(3,3,3-trifluoropropyl)succinamide (40); (2R,3S)-N-((3S)-9-chloro-5-(3-cyclopropylphenyl)-2-oxo-2,3-dihydro-1H-1,4-benzodiazepin-3-yl)-2,3-bis(3,3,3-trifluoropropyl)succinamide (41); (2R,3S)-N-((3S)-5-(3-chlorophenyl)-9-methoxy-2-oxo-2,3-dihydro-1H-1,4-benzodiazepin-3-yl)-2,3-bis(3,3,3-trifluoropropyl)succinamide (42); (2R,3S)-N-((3S)-5-(4-chlorophenyl)-9-methoxy-2-oxo-2,3-dihydro-1H-1,4-benzodiazepin-3-yl)-2,3-bis(3,3,3-trifluoropropyl)succinamide (43); (2R,3S)-N-((3S)-9-chloro-5-(3-methylphenyl)-2-oxo-2,3-dihydro-1H-1,4-benzodiazepin-3-yl)-2,3-bis(3,3,3-trifluoropropyl)succinamide (44); (2R,3S)-N-((3S)-5-(3-methylphenyl)-9-methoxy-2-oxo-2,3-dihydro-1H-1,4-benzodiazepin-3-yl)-2,3-bis(3,3,3-trifluoropropyl)succinamide (45); (2R,3S)-N-((3S)-5-(4-(hydroxymethyl)phenyl)-2-oxo-2,3-dihydro-1H-1,4-benzodiazepin-3-yl)-2,3-bis(3,3,3-trifluoropropyl)succinamide (46); (2R,3S)-N-((3S)-5-(2-methylphenyl)-2-oxo-2,3-dihydro-1H-1,4-benzodiazepin-3-yl)-2,3-bis(3,3,3-trifluoropropyl)succinamide (47); (2R,3S)-N-((3S)-5-(3-methylphenyl)-2-oxo-2,3-dihydro-1H-1,4-benzodiazepin-3-yl)-2,3-bis(3,3,3-trifluoropropyl)succinamide (48); (2R,3S)-N-((3S)-9-methoxy-2-oxo-5-(5-(trifluoromethyl)-2-pyridinyl)-2,3-dihydro-1H-1,4-benzodiazepin-3-yl)-2,3-bis(3,3,3-trifluoropropyl)succinamide (49); (2R,3S)-N-((3S)-5-(5-chloro-2-pyridinyl)-9-methoxy-2-oxo-2,3-dihydro-1H-1,4-benzodiazepin-3-yl)-2,3-bis(3,3,3-trifluoropropyl)succinamide (50); (2R,3S)-N-((3S)-5-(4-methoxyphenyl)-2-oxo-2,3-dihydro-1H-1,4-benzodiazepin-3-yl)-2,3-bis(3,3,3-trifluoropropyl)succinamide (51); (2R,3S)-N-((3S)-5-(4-methylphenyl)-2-oxo-2,3-dihydro-1H-1,4-benzodiazepin-3-yl)-2,3-bis(3,3,3-trifluoropropyl)succinamide (52); (2R,3S)-N-((3S)-5-(3-fluorophenyl)-9-(hydroxymethyl)-2-oxo-2,3-dihydro-1H-1,4-benzodiazepin-3-yl)-2,3-bis(3,3,3-trifluoropropyl)succinamide (53); ((3S)-3-(((2R,3S)-3-carbamoyl-6,6,6-trifluoro-2-(3,3,3-trifluoropropyl)hexanoyl)amino)-5-(3-fluorophenyl)-9-methyl-2-oxo-2,3-dihydro-1H-1,4-benzodiazepin-1-yl)methyl L-valinate (54); ((3S)-3-(((2R,3S)-3-carbamoyl-6,6,6-trifluoro-2-(3,3,3-trifluoropropyl)hexanoyl)amino)-5-(3-fluorophenyl)-9-methyl-2-oxo-2,3-dihydro-1H-1,4-benzodiazepin-1-yl)methyl L-alaninate (55); S-(((2S,3R)-6,6,6-trifluoro-3-(((3S)-5-(3-fluorophenyl)-9-methyl-2-oxo-2,3-dihydro-1H-1,4-benzodiazepin-3-yl)carbamoyl)-2-(3,3,3-trifluoropropyl)hexanoyl)amino)-L-cysteine (56); tert-butyl S-(((2S,3R)-6,6,6-trifluoro-3-(((3S)-5-(3-fluorophenyl)-9-methyl-2-oxo-2,3-dihydro-1H-1,4-benzodiazepin-3-yl)carbamoyl)-2-(3,3,3-trifluoropropyl)hexanoyl)amino)-L-cysteinate (57); methyl S-(((2S,3R)-6,6,6-trifluoro-3-(((3S)-5-(3-fluorophenyl)-9-methyl-2-oxo-2,3-dihydro-1H-1,4-benzodiazepin-3-yl)carbamoyl)-2-(3,3,3-trifluoropropyl) hexanoyl)amino)-L-cysteinate (58); ((3S)-3-(((2R,3S)-3-carbamoyl-6,6,6-trifluoro-2-(3,3,3-trifluoropropyl)hexanoyl)amino)-5-(3-fluorophenyl)-9-methyl-2-oxo-2,3-dihydro-1H-1,4-benzodiazepin-1-yl)methyl (4-(phosphonooxy)phenyl)acetate (59); and ((3S)-3-(((2R,3S)-3-carbamoyl-6,6,6-trifluoro-2-(3,3,3-trifluoropropyl)hexanoyl)amino)-5-(3-fluorophenyl)-9-methyl-2-oxo-2,3-dihydro-1H-1,4-benzodiazepin-1-yl)methyl L-valyl-L-valinate (60); and salts thereof.

In another embodiment, the present invention provides compositions comprising compounds represented by the structure of Formula (I):

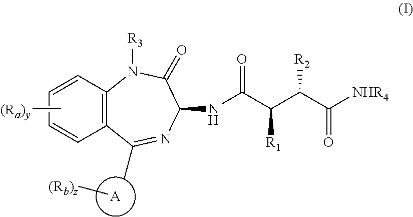

and/or at least one salt thereof, wherein:
R₁ is —CH₂CF₃ or —CH₂CH₂CF₃;
R₂ is —CH₂CF₃, —CH₂CH₂CF₃, or —CH₂CH₂CH₂CF₃;
R₃ is H, —CH₃ or Rₓ;
R₄ is H or R_{y};
Rₓ
is: —CH₂OC(O)CH(CH₃)NH₂, —CH₂OC(O)CH(NH₂)CH(CH₃)₂, —CH₂OC(O)CH((CH(CH₃)₂)NHC(O)CH(NH₂)CH(CH₃)₂,

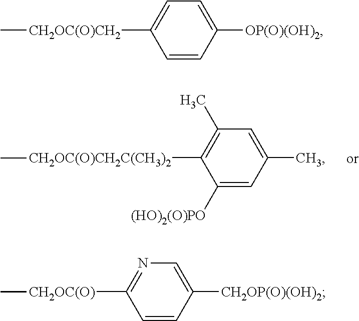

R_{y} is: —SCH₂CH(NH₂)C(O)OH, —SCH₂CH(NH₂)C(O)OH₃,
or —SCH₂CH(NH₂)C(O)OC(CH₃)₃;
Ring A is phenyl or pyridinyl;
each Rₐ is independently F, Cl, —CN, —OCH₃, C₁₋₃ alkyl, —CH₂OH, —CF₃, cyclopropyl, —OCH₃, —O(cyclopropyl) and/or —NHCH₂CH₂OCH₃;
each R_{b} is independently F, Cl, —CH₃, —CH₂OH, —CF₃, cyclopropyl, and/or —OCH₃;
y is zero, 1 or 2; and
z is zero, 1, or 2
provided that if Ring A is phenyl, z is zero, and y is 1 or 2 then at least one Rₐ is C₁₋₃ alkyl, —CH₂OH, —CF₃, cyclopropyl, or —O(cyclopropyl);
provided that if R₃ is Rₓ then R₄ is H; and
provided that if R₄ is R_{y} then R₃ is H or —CH₃.

In another embodiment, the structure as described hereinabove comprises one or more of the following provisos: provided that if Ring A is phenyl, z is zero, and y is 1 or 2 then at least one Rₐ is C₁₋₃ alkyl, —CH₂OH, —CF₃, cyclopropyl, or —O(cyclopropyl); provided that if R₃ is Rₓ then R₄ is H; and provided that if R₄ is R_{y} then R₃ is H or —CH₃.

In another embodiment, the present invention provides compositions comprising compounds represented by the following structure:

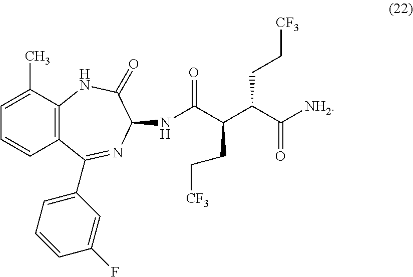 [SMILES: Cc1cccc2c1NC(=O)[C@@H](NC(=O)[C@H](CCC(F)(F)F)[C@H](CCC(F)(F)F)C(N)=O)N=C2c1cccc(F)c1]

In another embodiment, the compounds as described herein comprise prodrugs of one or more of the compounds.

U.S. Pat. No. 9,273,014, which is incorporated by reference herein in its entirety, discloses various compounds of Formula (I):

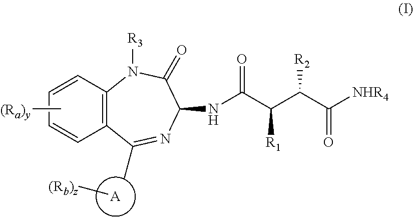

and/or at least one salt thereof, wherein:
R₁ is —CH₂CH₂CF₃;
R₂ is —CH₂CH₂CF₃ or —CH₂CH₂CH₂CF₃;
R₃ is H, —CH₃, or Rₓ;
R₄ is H or R_{y};
Rₓ
is: —CH₂OC(O)CH(CH₃)NH₂, —CH₂OC(O)CH(NH₂)CH(CH₃)₂, —CH₂OC(O)CH((CH(CH₃)₂)NHC(O)CH(NH₂)CH(CH₃)₂,

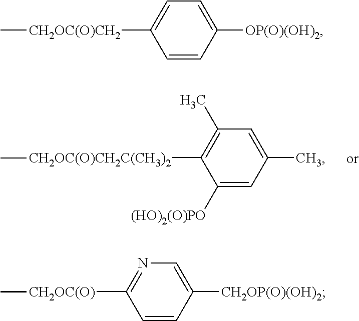

R_{y} is: —SCH₂CH(NH₂)C(O)OH, —SCH₂CH(NH₂)C(O)OCH₃,
or —SCH₂CH(NH₂)C(O)OC(CH₃)₃;
Ring A is phenyl or pyridinyl;
each Rₐ is independently Cl, C₁₋₃ alkyl, —CH₂OH, —CF₃, cyclopropyl, —OCH₃, and/or —O(cyclopropyl);
each R_{b} is independently F, Cl, —CH₃, —CH₂OH, —CF₃, cyclopropyl, and/or —OCH₃;
y is zero, 1, or 2; and
z is 1 or 2.

U.S. Pat. No. 9,273,014 also discloses the compound of Formula (22):

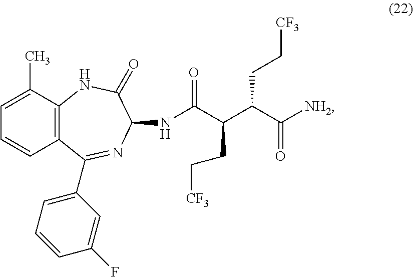 [SMILES: Cc1cccc2c1NC(=O)[C@@H](NC(=O)[C@H](CCC(F)(F)F)[C@H](CCC(F)(F)F)C(N)=O)N=C2c1cccc(F)c1]

which, in one embodiment, has the chemical name (2R,3S)-N-((3S)-5-(3-fluorophenyl)-9-methyl-2-oxo-2,3-dihydro-1H-1,4-benzodiazepin-3-yl)-2,3-bis(3,3,3-trifluoropropyl)succinamide. U.S. Pat. No. 9,273,014 also discloses a process for synthesizing the compounds as well as other compounds of Formula (I), which are to be considered as part of the present invention.

U.S. Pat. No. 8,629,136, which is incorporated by reference herein in its entirety, discloses compounds of Formula (III):

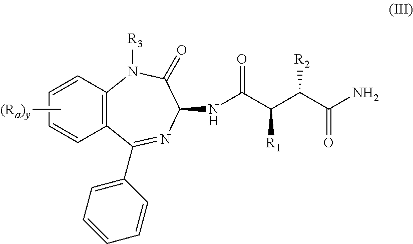

and/or at least one salt thereof, wherein:
R₃ is H or —CH₃; and
each Rₐ is independently F, Cl, —CN, —OCH₃ and/or —NHCH₂CH₂OCH₃.

U.S. Pat. No. 8,629,136 also discloses the structure of Compound (1):

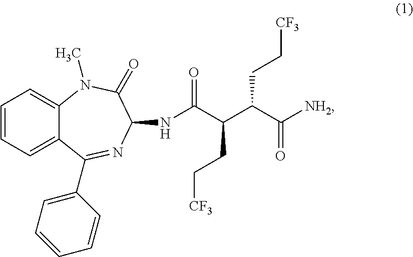 [SMILES: CN1C(=O)[C@@H](NC(=O)[C@H](CCC(F)(F)F)[C@H](CCC(F)(F)F)C(N)=O)N=C(c2ccccc2)c2ccccc21]

which, in one embodiment, has the chemical name (2R,3S)-N-((3S)-1-methyl-2-oxo-5-phenyl-2,3-dihydro-1H-1,4-benzodiazepin-3-yl)-2,3-bis(3,3,3-trifluoropropyl)succinamide. In one embodiment, the compounds are Notch inhibitors. U.S. Pat. No. 8,629,136 discloses a process for synthesizing the compounds as well as other compounds of Formula (I), which are to be considered as part of the present invention.

The present invention may be embodied in other specific forms without departing from the spirit or essential attributes thereof. This invention encompasses all combinations of the aspects and/or embodiments of the invention noted herein. It is understood that any and all embodiments of the present invention may be taken in conjunction with any other embodiment or embodiments to describe addition more embodiments. It is also to be understood that each individual element of the embodiments is meant to be combined with any and all other elements from any embodiment to describe an additional embodiment.

### Combined Treatments

In one embodiment, the present invention provides compositions comprising compounds represented by the structure of Formula (I) as described herein as monotherapy or in a combination therapy with one or more anti-cancer, cytotoxic, or therapeutic agents.

In another embodiment, the present invention provides compositions comprising compounds represented by the structure of Formula (I) as described herein as monotherapy or in a combination therapy with one or more chemotherapeutic agents.

In one embodiment, the present invention provides a composition comprising one or more compounds represented by the structure of Formula (III) as monotherapy or in a combination therapy with one or more anti-cancer, cytotoxic, or therapeutic agents:

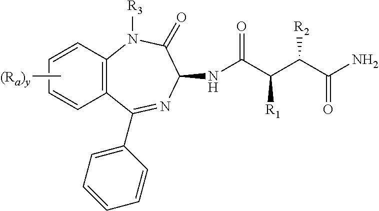

or prodrugs or salts thereof; wherein:
R₁ is —CH₂CF₃ or —CH₂CH₂CF₃;
R₂ is —CH₂CF₃, —CH₂CH₂CF₃, or —CH₂CH₂CH₂CF₃;
R₃ is H or —CH₃;
each Rₐ is independently F, Cl, —CN, —OCH₃, and/or —NHCH₂CH₂OCH₃; and
y is zero, 1, or 2.

In one embodiment, the present invention provides a composition comprising one or more compounds represented by the structure of Formula (III) as monotherapy or in a combination therapy with one or more chemotherapeutic agents:

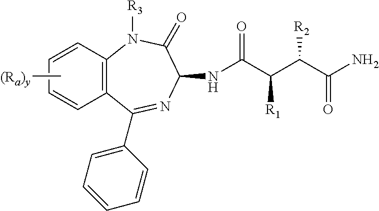

or prodrugs or salts thereof; wherein:
R₁ is —CH₂CF₃ or —CH₂CH₂CF₃;
R₂ is —CH₂CF₃, —CH₂CH₂CF₃, or —CH₂CH₂CH₂CF₃;
R₃ is H or —CH₃;
each Rₐ is independently F, Cl, —CN, —OCH₃, and/or —NHCH₂CH₂OCH₃; and y is zero, 1, or 2.

In one embodiment, the chemotherapeutic agent comprises eribulin. In one embodiment, eribulin is administered at a dose of approximately 1.4 mg/m², in one embodiment, over 2-5 minutes. In one embodiment, eribulin is administered on days 1 and 9 of a 21-day cycle. In another embodiment, eribulin is administered at a dose of approximately 1.1 mg/m². In another embodiment, eribulin is administered at a dose of approximately 0.7 mg/m². In another embodiment, eribulin is administered at a dose of 0.5-5 mg/m². In another embodiment, eribulin is administered at a dose of 1-4 mg/m². In another embodiment, eribulin is administered at a dose of 0.5-2.5 mg/m². In another embodiment, eribulin is administered at a dose of 1-1.5 mg/m². In another embodiment, eribulin is administered at a dose of 0.7-1.4 mg/m². In one embodiment, eribulin is administered intravenously.

In one embodiment, eribulin is a fully synthetic macrocyclic ketone analogue of the marine natural product halichondrin B. In one embodiment, eribulin is a mitotic inhibitor. In one embodiment, eribulin comprises cytotoxic and non-cytotoxic effects. In one embodiment, eribulin is an inhibitor of microtubule dynamics. Thus, in one embodiment, the methods described herein may include the step of administering a composition comprising a synthetic analogue of halichondrin B, a mitotic inhibitor, an inhibitor of microtubule dynamics, or a combination thereof.

Thus, in one embodiment, the present invention provides a method of inhibiting tumor growth in a subject having breast cancer, comprising the step of administering to said subject a first composition comprising a cytotoxic agent and a second composition comprising one or more compounds represented by the structure of Formula (I) or Formula (III) as described herein. In one embodiment, the cytotoxic agent comprises eribulin. In one embodiment, the breast cancer comprises TNBC.

In another embodiment, the present invention provides a method of reducing tumor size in a subject having breast cancer, comprising the step of administering to said subject a first composition comprising a cytotoxic agent and a second composition comprising one or more compounds represented by the structure of Formula (I) or Formula (III) as described herein. In one embodiment, the cytotoxic agent comprises eribulin. In one embodiment, the breast cancer comprises TNBC.

Thus, in one embodiment, the present invention provides a method of suppressing tumor growth in a subject having breast cancer, comprising the step of administering to said subject a first composition comprising a cytotoxic agent and a second composition comprising one or more compounds represented by the structure of Formula (I) or Formula (III) as described herein. In one embodiment, the cytotoxic agent comprises eribulin. In one embodiment, the breast cancer comprises TNBC.

In one embodiment, the present invention provides a method of inhibiting tumor outgrowth in a subject, which in one embodiment, occurs in the subject after around of treatment and subsequent treatment withdrawal. In one embodiment, the round of treatment is the first round of treatment. In another embodiment, the round of treatment is the second, third, fourth, or fifth round of treatment. In one embodiment, the tumor growth inhibition occurs in the 2nd treatment cycle for said subject.

In one embodiment, the subject is administered the same composition or combination of compositions in the first and second treatment cycles. In another embodiment, the subject is administered different compositions or combination of compositions in the first and second treatment cycles.

In one embodiment, compositions of the present invention or for use in the methods of the present invention comprise one or more cancer therapeutic agents in a combination therapy with one or more bisfluoroalkyl-1,4-benzodiazepinone compounds described hereinabove.

In treating cancer, a combination of chemotherapeutic agents and/or other treatments (e.g., radiation therapy) is often advantageous. An additional agent may have the same or different mechanism of action than the primary therapeutic agents. For example, drug combinations may be employed wherein the two or more drugs being administered act in different manners or in different phases of the cell cycle, and/or where the two or more drugs have nonoverlapping toxicities or side effects, and/or where the drugs being combined each has a demonstrated efficacy in treating the particular disease state manifested by the patient.

In another embodiment, the present invention provides a composition comprising one or more compounds represented by the structure of Formula (I) as described herein in combination with eribulin.

In another embodiment, the present invention provides a composition comprising one or more compounds represented by the structure of Formula (I) as described herein in combination with vinorelbine.

In another embodiment, the present invention provides a composition comprising one or more compounds represented by the structure of Formula (I) as described herein in combination with FOLFRI. In one embodiment, FOLFIRI comprises folinic acid (leucovorin), fluorouracil (5-FU) and irinotecan (Camptosar). In another embodiment, the present invention provides a composition comprising one or more compounds represented by the structure of Formula (I) as described herein and folinic acid (leucovorin), fluorouracil (5-FU), irinotecan (Camptosar), or a combination thereof.

In one embodiment, a composition of the present invention comprises one or more compounds represented by the structure of Formula (I) as described herein and one or more targeted therapeutics. In one embodiment, said targeted therapeutic comprises an inhibitor of mammalian target of rapamycin (mTOR). In one embodiment, the mTOR inhibitor comprises Everolimus. In another embodiment, the mTOR inhibitor comprises sirolimus (rapamycin). In another embodiment, the mTOR inhibitor comprises temsirolimus.

In another embodiment, the mTOR inhibitor comprises a dual mammalian target of rapamycin/phosphoinositide 3-kinase inhibitor, which in one embodiment, comprises NVP-BEZ235 (dactolisib), GSK2126458, XL765, or a combination thereof.

In another embodiment, the mTOR inhibitor comprises a second generation mTOR inhibitor, which, in one embodiment, comprises AZD8055, INK128/MLN0128, OSI027, or a combination thereof.

In another embodiment, the mTOR inhibitor comprises a third generation mTOR inhibitor, which, in one embodiment, comprises RapaLinks.

In one embodiment, a composition of the present invention comprises one or more compounds represented by the structure of Formula (I) as described herein in combination with an mTOR inhibitor and a chemotherapeutic drug. In one embodiment, the mTOR inhibitor comprises everolimus. In one embodiment, the chemotherapeutic drug comprises cisplatin.

In one embodiment, a composition of the present invention comprises one or more compounds represented by the structure of Formula (I) as described herein in combination with a PARP (poly ADP-ribose polymerase) inhibitor.

In another embodiment, a composition of the present invention comprises one or more compounds represented by the structure of Formula (I) as described herein and a polyfunctional alkylating agent. In one embodiment, the polyfunctional alkylating agent comprises a Nitrosourea, Mustard, Nitrogen Mustard, Methanesulphonate, Busulphan, Ethylenimine, or a combination thereof.

In another embodiment, a composition of the present invention comprises one or more compounds represented by the structure of Formula (I) as described herein in combination with steroids.

In another embodiment, a composition of the present invention comprises one or more compounds represented by the structure of Formula (I) as described herein in combination with bisphosphonates.

In another embodiment, a composition of the present invention comprises one or more compounds represented by the structure of Formula (I) as described herein in combination with cancer growth blockers.

In another embodiment, a composition of the present invention comprises one or more compounds represented by the structure of Formula (I) as described herein in combination with proteasome inhibitors.

In another embodiment, a composition of the present invention comprises one or more compounds represented by the structure of Formula (I) as described herein in combination with one or more interferons.

In another embodiment, a composition of the present invention comprises one or more compounds represented by the structure of Formula (I) as described herein in combination with one or more interleukins.

In another embodiment, the present invention provides a composition comprising one or more compounds represented by the structure of Formula (I) as described herein and an alkylating drug. In one embodiment, the alkylating drug comprises Procarbazine (Matulane), Dacarbazine (DTIC), Altretamine (Hexalen), or a combination thereof.

In another embodiment, the present invention provides a composition comprising one or more compounds represented by the structure of Formula (I) as described herein and an alkylating-like drug. In one embodiment, the alkylating-like drug comprises Cisplatin (Platinol).

In another embodiment, the present invention provides a composition comprising one or more compounds represented by the structure of Formula (I) as described herein and an antimetabolite. In one embodiment, the antimetabolite comprises an antifolic acid compound (Methotrexate), an amino acid antagonists (Azaserine), or a combination thereof.

In another embodiment, the present invention provides a composition comprising one or more compounds represented by the structure of Formula (I) as described herein and a purine antagonist. In one embodiment, the purine antagonist comprises Mercaptopurine (6-MP), Thioguanine (6-TG), Fludarabine Phosphate, Cladribine (Leustatin), Pentostatin (Nipent), or a combination thereof.

In another embodiment, the present invention provides a composition comprising one or more compounds represented by the structure of Formula (I) as described herein and a pyrimidine antagonist. In one embodiment, the pyrimidine antagonist comprises Fluorouracil (5-FU), Cytarabine (ARA-C), Azacitidine, or a combination thereof.

In another embodiment, the present invention provides a composition comprising one or more compounds represented by the structure of Formula (I) as described herein and a plant alkaloid. In one embodiment, the plant alkaloid comprises Vinblastine (Velban), Vincristine (Oncovin), Etoposide (VP-16, VePe-sid), Teniposide (Vumon), Topotecan (Hycamtin), Irinotecan (Camptosar), Paclitaxel (Taxol), Docetaxel (Taxotere), or a combination thereof.

In another embodiment, the present invention provides a composition comprising one or more compounds represented by the structure of Formula (I) as described herein and an antibiotic. In one embodiment, the antibiotic comprises Anthracyclines, Doxorubicin (Adriamycin, Rubex, Doxil), Daunorubicin (DaunoXome), Dactinomycin (Cosmegen), Idarubincin (Idamycin), Plicamycin (Mithramycin), Mitomycin (Mutamycin), Bleomycin (Blenoxane), or a combination thereof.

In another embodiment, the present invention provides a composition comprising one or more compounds represented by the structure of Formula (I) as described herein in combination with a cancer vaccine. In another embodiment, the present invention provides a composition comprising one or more compounds represented by the structure of Formula (I) as described herein and an immunotherapeutic. In one embodiment, the immunotherapeutic comprises a monoclonal antibody. In one embodiment, the monoclonal antibody comprises an anti-PD-1 antibody, which in one embodiment comprises nivolumab.

In another embodiment, the monoclonal antibody comprises alemtuzumab (Campath®), trastuzumab (Herceptin®), Bevacizumab (Avastin®), Cetuximab (Erbitux®), or a combination thereof. In another embodiment, the monocolonal antibody comprises a radiolabeled antibody, which, in one embodiment, comprises britumomab, tiuxetan (Zevalin®), or a combination thereof. In another embodiment, the monocolonal antibody comprises a chemolabeled antibody, which in one embodiment comprises Brentuximab vedotin (Adcetris®), Ado-trastuzumab entansine (Kadcyla®, also called TDM-1), denileukin diftitox (Ontak®), or a combination thereof. In another embodiment, the monocolonal antibody comprises a bispecific antibody, which in one embodiment, comprises blinatumomab (Blincyto).

In another embodiment, the present invention provides a composition comprising one or more compounds represented by the structure of Formula (I) as described herein in combination with an antibody-drug conjugate (ADC), which in one embodiment, comprises an antibody linked to a biologically active cytotoxic (in another embodiment, anticancer) drug (in another embodiment, payload). In one embodiment, the antibody is a monoclonal antibody. In one embodiment, the antibody is linked to the cytotoxic agent via a chemical linker. In one embodiment, the ADC is designed to selectively deliver the cytotoxic agent directly to the target cancer cells.

In one embodiment, the ADC comprises Sacituzumab govitecan. In another embodiment, the ADC comprises Gemtuzumab ozogamicin, Brentuximab vedotin, Trastuzumab emtansine, Inotuzumab ozogamicin, Polatuzumab vedotin-piiq, Enfortumab vedotin, Trastuzumab deruxtecan, Sacituzumab govitecan, or a combination thereof. In another embodiment, the ADS comprises a Trop-2 antibody, a topoisomerase inhibitor, or a combination thereof.

In another embodiment, the present invention provides a composition comprising one or more compounds represented by the structure of Formula (I) as described herein in combination with a hormonal therapy. In another embodiment, the present invention provides a composition comprising one or more compounds represented by the structure of Formula (I) as described herein and a hormonal agent. In one embodiment, the hormonal agent comprises Tamoxifen (Nolvadex), Flutamide (Eulexin), Gonadotropin-Releasing Hormone Agonists, (Leuprolide and Goserelin (Zoladex)), Aromatase Inhibitors, Aminoglutethimide, Anastrozole (Arimidex), or a combination thereof.

In another embodiment, the present invention provides a composition comprising one or more compounds represented by the structure of Formula (I) as described herein and Amsacrine, Hydroxyurea (Hydrea), Asparaginase (El-spar), Mitoxantrone (Novantrone), Mitotane, Retinoic Acid Derivatives, Bone Marrow Growth Factors, Amifostine, or a combination thereof.

In another embodiment, the present invention provides a composition comprising one or more compounds represented by the structure of Formula (I) as described herein in combination with an agent that inhibits one or more cancer stem cell pathways. In one embodiment, such agent comprises an inhibitor of Hedgehog, WNT, BMP, or a combination thereof.

In one embodiment, said anti-cancer, cytotoxic, or therapeutic agent comprises a BCMA-targeted chimeric antigen receptor T-cell immunotherapeutic, p53-HDM2 inhibitor, c-MET inhibitor, BCR-ABL inhibitor, Anti-interleukin-1 beta monoclonal antibody, EGFR mutation modulator, PI3K-alpha inhibitor, JAK1/2 inhibitor, Cortisol synthesis inhibitor, Thrombopoietin, P-selectin inhibitor receptor agonist, Anti-CD20 monoclonal antibody, Anti-PD-1 monoclonal antibody, Signal transduction inhibitor, CDK4/6 inhibitor, BRAF inhibitor+MEK inhibitor, CD19-targeted chimeric antigen receptor T-cell immunotherapeutic, Somatostatin analogue, or a combination thereof. In one embodiment, said anti-cancer agent comprises capmatinib, asciminib, canakinumab, alpelisib, ruxolitinib, osilodrostat, eltrombopag, crizanlizumab, ofatumumab, spartalizumab, midostaurin, ribociclib, dabrafenib+trametinib, tisagenlecleucel, everolimus, pasireotide, or a combination thereof.

In another embodiment, the present invention provides a composition comprising one or more compounds represented by the structure of Formula (I) as described herein in combination with a hematopoietic stem cell transplant approach.

In another embodiment, the present invention provides a composition comprising one or more compounds represented by the structure of Formula (I) as described herein in combination with isolated infusion approaches. In one embodiment, the isolated infusion approach comprises infusion of chemotherapy into a specific tissue in order to deliver a very high dose of chemotherapy to tumor sites without causing overwhelming systemic damage.

In another embodiment, the present invention provides a composition comprising one or more compounds represented by the structure of Formula (I) as described herein in combination with targeted delivery mechanisms. In one embodiment, the targeted delivery mechanism increases effective levels of chemotherapy for tumor cells while reducing effective levels for other cells for increased tumor specificity and/or reduced toxicity. In one embodiment, targeted delivery mechanisms comprise a traditional chemotherapeutic agent, or a radioisotope or an immune stimulating factor.

In another embodiment, the present invention provides a composition comprising one or more compounds represented by the structure of Formula (I) as described herein in combination with nanoparticles. In one embodiment, nanoparticles are used as a vehicle for poorly-soluble agents such as paclitaxel. In one embodiment, nanoparticles made of magnetic material can also be used to concentrate agents at tumour sites using an externally applied magnetic field.

In another embodiment, the present invention provides a composition comprising one or more compounds represented by the structure of Formula (I) as described herein in combination with an agent for treating Triple Negative Brest Cancer (TNBC). In one embodiment, said agent for treating TNBC comprises Axitinib, Bortezomib (Velcade), Bortezomib+doxorubicin, Cetuximab, Cetuximab+Intensity modulated radiation therapy (IMRT), Cetuximab+RT+cisplatin, Cetuximab+cisplatin+5-FU, Chidamide (CS055/HBI-8000), Cetuximab & Carbon Ion, Cisplatin, cisplatin & 5-FU, Cisplatin & Doxorubicin & Bleomycin, Cisplatin & Doxorubicin & Cyclophosphamide, Dasatinib, Dovitinib, Epirubicin, Gefitinib, Gemcitabine, Gemcitabine & Cisplatin, Imatinib, Imatinib+cisplatin, Lapatinib, Mitoxanthrone, MK 2206, Nelfinavir, Paclitaxel, Paclitaxel & Carboplatin, Panitumumab & Radiotherapy, PF-00562271, PF-00299804 & Figitumumab PX-478, PX-866, Regorafenib, Sonepcizumab, Sorafenib, Sunitinib, Vinorelbine, Vinorelbine & Cisplatin, Vorinostat, XL147 & Erlotinib, XL647, or combinations thereof.

In another embodiment, the present invention provides a composition comprising one or more compounds represented by the structure of Formula (I) as described herein in combination with pembrolizumab, docetaxel, nivolumab and ipilimumab, PSMA-PET Imaging, chidamide, APG-115, HDM201, DS-3032b, LY3039478, or a combination thereof.

In another embodiment, said agent for treating triple-negative breast cancer comprises PARP (poly ADP-ribose polymerase) inhibitors such as olaparib, VEGF (vascular endothelial growth factor) inhibitors such as bevacizumab, EGFR (epidermal growth factor receptor)-targeted therapies such as cetuximab, or a combination thereof.

In one embodiment, a method is provided for treating cancer comprising administering to a subject in need thereof a composition as described herein and administering one or more anti-cancer agents.

In one embodiment, the phrase “anti-cancer agent” refers to a drug selected from any one or more of the following: alkylating agents (including mustard, nitrogen mustards, methanesulphonate, busulphan, alkyl sulfonates, nitrosoureas, ethylenimine derivatives, and triazenes or combinations thereof); anti-angiogenics (including matrix metalloproteinase inhibitors); antimetabolites (including adenosine deaminase inhibitors, folic acid antagonists, purine analogues, and pyrimidine analogues); antibiotics or antibodies (including monoclonal antibodies, CTLA-4 antibodies, anthracyclines); aromatase inhibitors; cell-cycle response modifiers; enzymes; farnesyl-protein transferase inhibitors; hormonal and antihormonal agents and steroids (including synthetic analogs, glucocorticoids, estrogens/anti-estrogens [e.g., SERMs], androgens/anti-androgens, progestins, progesterone receptor agonists, and luteinizing hormone-releasing [LHRH] agonists and antagonists); insulin-like growth factor (IGF)/insulin-like growth factor receptor (IGFR) system modulators (including IGFR1 inhibitors); integrin-signaling inhibitors; kinase inhibitors (including multi-kinase inhibitors and/or inhibitors of Src kinase or Src/ab1, cyclin dependent kinase [CDK] inhibitors, panHer, Her-1 and Her-2 antibodies, VEGF inhibitors, including anti-VEGF antibodies, EGFR inhibitors, PARP (poly ADP-ribose polymerase) inhibitors, mitogen-activated protein [MAP] inhibitors, MET inhibitors, MEK inhibitors, Aurora kinase inhibitors, PDGF inhibitors, and other tyrosine kinase inhibitors or serine/threonine kinase inhibitors; microtubule-disruptor agents, such as ecteinascidins or their analogs and derivatives; microtubule-stabilizing agents such as taxanes, Platinum-based antineoplastic drugs (platins) such as cisplatin, carboplatin, oxaliplatin, nedaplatin, triplatin tetranitrate, phenanthriplatin, picoplatin and satraplatin and the naturally-occurring epothilones and their synthetic and semi-synthetic analogs; microtubule-binding, destabilizing agents (including vinca alkaloids); topoisomerase inhibitors; prenyl-protein transferase inhibitors; platinum coordination complexes; signal transduction inhibitors; and other agents used as anti-cancer and cytotoxic agents such as biological response modifiers, growth factors, and immune modulators. In another embodiment, “anti-cancer agent” comprises taxanes, platins, or a combination thereof.

In another embodiment, the present invention provides a composition comprising one or more compounds represented by the structure of Formula (I) as described herein in combination with any one or more of the following: Revlimid, Avastin, Herceptin, Rituxan, Opdivo, Gleevec, Imbruvica, Velcade, Zytiga, Xtandi, Alimta, Gadasil, Ibrance, Perjeta, Tasigna, Xgeva, Afinitor, Jakafi, Tarceva, Keytruda, Sutent, Yervoy, Nexavar, Zoladex, Erbitux, Dazalex, Xeloda, Gazyva, Venclexta, and Tecentriq.

In another embodiment, the present invention provides a composition comprising one or more compounds represented by the structure of Formula (I) as described herein in combination with any one or more of the following: abemaciclib, epacadostat, apalutamide, Carfilzomib, Crizotinib (PF-02341066), GDC-0449 (vismodegib), OncoVex, PLX4032 (RG7204), Ponatinib, SGN-35 (brentuximab vedotin), Tivozanib (AV-951), T-DM1 (Trastuzumab-DM1), and XL184 (cabozantinib).

Accordingly, the compositions of the present invention may be administered in combination with other anti-cancer treatments useful in the treatment of cancer or other proliferative diseases. The invention herein further comprises use of the compositions of the present invention in preparing medicaments for the treatment of cancer, and/or it comprises the packaging of the compositions of the present invention together with instructions that the compositions be used in combination with other anti-cancer or cytotoxic agents and treatments for the treatment of cancer.

In one embodiment, any of the methods as described herein comprises the step of administering to a subject a composition comprising compounds represented by the structure of Formula (I) as described herein as monotherapy or in a combination therapy with one or more anti-cancer agents. In another embodiment, any of the methods as described herein comprises the step of administering to a subject a composition comprising compounds represented by the structure of Formula (I) as described herein as monotherapy or in a combination therapy with one or more chemotherapeutic agents.

In another embodiment, any of the methods as described herein comprises the step of administering to a subject a composition comprising compounds represented by the structure of Formula (III) as described herein as monotherapy or in a combination therapy with one or more anti-cancer agents. In another embodiment, any of the methods as described herein comprises the step of administering to a subject a composition comprising compounds represented by the structure of Formula (III) as described herein as monotherapy or in a combination therapy with one or more chemotherapeutic agents.

In one embodiment, the anti-cancer or chemotherapeutic agent(s) in the methods of the present invention are administered to the subject in a single composition with a compound represented by the structure of Formula (I) or a compound represented by the structure of Formula (III). In another embodiment, the anti-cancer or chemotherapeutic agent(s) are administered to the subject in separate compositions from the composition comprising a compound represented by the structure of Formula (I) or a compound represented by the structure of Formula (III). In one embodiment, the separate compositions are administered to the subject at the same time. In another embodiment, the separate compositions are administered to the subject at separate times, at separate sites of administration, or a combination thereof.

In one embodiment, a method is provided for treating cancer comprising administering to a subject in need thereof a compound of Formula (I), administering a glucocorticoid; and optionally, administering one or more additional anti-cancer agents. An example of a suitable glucocorticoid is dexamethasone.

In another embodiment, a method is provided herein comprising administering to a subject a composition comprising a compound of Formula (I) and a composition comprising a corticosteroid.

In one embodiment, the corticosteroid comprises a glucocorticoid. In one embodiment, the glucocorticoid comprises dexamethasone. In one embodiment, the dexamethasone is administered prophylactically. In one embodiment, the dexamethasone is administered every 4-6 hours for up to 72 hours. In one embodiment, the dexamethasone is administered at a dose of 4-8 mg. In one embodiment, the dexamethasone is administered orally or intravenously.

In one embodiment, a method is provided for treating cancer comprising administering to a subject in need thereof a compound of Formula (I); administering cisplatin; and optionally, administering one or more additional anti-cancer agents.

In one embodiment, a method is provided for treating cancer comprising administering to a subject in need thereof a compound of Formula (I); administering dasatinib; and optionally, administering one or more additional anti-cancer agents.

In one embodiment, a method is provided for treating cancer comprising administering to a subject in need thereof a compound of Formula (I); administering paclitaxel; and optionally, administering one or more additional anti-cancer agents.

In one embodiment, a method is provided for treating cancer comprising administering to a subject in need thereof a compound of Formula (I); administering tamoxifen; and optionally, administering one or more additional anti-cancer agents.

In one embodiment, a method is provided for treating cancer comprising administering to a subject in need thereof a compound of Formula (I), administering carboplatin; and optionally, administering one or more additional anti-cancer agents.

The compounds of the present invention can be formulated or co-administered with other therapeutic agents that are selected for their particular usefulness in addressing side effects associated with the aforementioned conditions. For example, compounds of the invention may be formulated with agents to prevent nausea, hypersensitivity and gastric irritation, such as antiemetics, and H₁ and H₂ antihistaminics.

In one embodiment, pharmaceutical compositions are provided comprising a compound of Formula (I) or prodrug thereof; one or more additional agents selected from a kinase inhibitory agent (small molecule, polypeptide, and antibody), an immunosuppressant, an anti-cancer agent, an anti-viral agent, anti-inflammatory agent, antifungal agent, antibiotic, or an anti-vascular hyperproliferation compound; and any pharmaceutically acceptable carrier, adjuvant or vehicle.

In one embodiment, a combined treatment is administered to a subject having a TNBC tumor which lacks a GOF mutation. In one embodiment, a combined treatment is administered to a subject having a TNBC tumor which does not comprise a known GOF mutation.

The above other therapeutic agents, when employed in combination with the compounds of the present invention, may be used, for example, in those amounts indicated in the Physicians' Desk Reference (PDR) or as otherwise determined by one of ordinary skill in the art.

### Pharmaceutical Compositions

### Formulations

Also embraced within this invention is a class of pharmaceutical compositions comprising the compound of Formula (I) and one or more non-toxic, pharmaceutically-acceptable carriers and/or diluents and/or adjuvants (collectively referred to herein as “carrier” materials) and, if desired, other active ingredients.

The compounds of Formula (I) may be administered by any suitable route, preferably in the form of a pharmaceutical composition adapted to such a route, and in a dose effective for the treatment intended. The compounds and compositions of the present invention may, for example, be administered in dosage unit formulations containing conventional pharmaceutically acceptable carriers, adjuvants, and vehicles. For example, the pharmaceutical carrier may contain a mixture of mannitol or lactose and microcrystalline cellulose. The mixture may contain additional components such as a lubricating agent, e.g., magnesium stearate and a disintegrating agent such as crospovidone. The carrier mixture may be filled into a gelatin capsule or compressed as a tablet. The pharmaceutical composition may be administered as an oral dosage form or an infusion, for example.

For oral administration, the pharmaceutical composition may be in the form of, for example, a tablet, capsule, liquid capsule, suspension, or liquid. The pharmaceutical composition is preferably made in the form of a dosage unit containing a particular amount of the active ingredient. For example, the pharmaceutical composition may be provided as a tablet or capsule comprising an amount of active ingredient in the range of from about 1 to 2000 mg, preferably from about 1 to 500 mg, and more preferably from about 5 to 150 mg. A suitable daily dose for a human or other mammal may vary widely depending on the condition of the patient and other factors, but can be determined using routine methods.

Any pharmaceutical composition contemplated herein can, for example, be delivered orally via any acceptable and suitable oral preparations. Exemplary oral preparations, include, but are not limited to, for example, tablets, troches, lozenges, aqueous and oily suspensions, dispersible powders or granules, emulsions, hard and soft capsules, liquid capsules, syrups, and elixirs. Pharmaceutical compositions intended for oral administration can be prepared according to any methods known in the art for manufacturing pharmaceutical compositions intended for oral administration. In order to provide pharmaceutically palatable preparations, a pharmaceutical composition in accordance with the invention can contain at least one agent selected from sweetening agents, flavoring agents, coloring agents, demulcents, antioxidants, and preserving agents.

A tablet can, for example, be prepared by admixing at least one compound of Formula (I) with at least one non-toxic pharmaceutically acceptable excipient suitable for the manufacture of tablets. Exemplary excipients include, but are not limited to, for example, inert diluents, such as, for example, calcium carbonate, sodium carbonate, lactose, calcium phosphate, and sodium phosphate; granulating and disintegrating agents, such as, for example, microcrystalline cellulose, sodium croscarmellose, corn starch, and alginic acid; binding agents, such as, for example, starch, gelatin, polyvinyl-pyrrolidone, and acacia; and lubricating agents, such as, for example, magnesium stearate, stearic acid, and talc. Additionally, a tablet can either be uncoated, or coated by known techniques to either mask the bad taste of an unpleasant tasting drug, or delay disintegration and absorption of the active ingredient in the gastrointestinal tract thereby sustaining the effects of the active ingredient for a longer period. Exemplary water soluble taste masking materials, include, but are not limited to, hydroxypropyl-methylcellulose and hydroxypropyl-cellulose. Exemplary time delay materials, include, but are not limited to, ethyl cellulose and cellulose acetate butyrate.

Hard gelatin capsules can, for example, be prepared by mixing at least one compound of Formula (I) with at least one inert solid diluent, such as, for example, calcium carbonate; calcium phosphate; and kaolin.

Soft gelatin capsules can, for example, be prepared by mixing at least one compound of Formula (I) with at least one water soluble carrier, such as, for example, polyethylene glycol; and at least one oil medium, such as, for example, peanut oil, liquid paraffin, and olive oil.

An aqueous suspension can be prepared, for example, by admixing at least one compound of Formula (I) with at least one excipient suitable for the manufacture of an aqueous suspension. Exemplary excipients suitable for the manufacture of an aqueous suspension, include, but are not limited to, for example, suspending agents, such as, for example, sodium carboxymethylcellulose, methylcellulose, hydroxypropylmethyl-cellulose, sodium alginate, alginic acid, polyvinyl-pyrrolidone, gum tragacanth, and gum acacia; dispersing or wetting agents, such as, for example, a naturally-occurring phosphatide, e.g., lecithin; condensation products of alkylene oxide with fatty acids, such as, for example, polyoxyethylene stearate; condensation products of ethylene oxide with long chain aliphatic alcohols, such as, for example heptadecaethylene-oxycetanol; condensation products of ethylene oxide with partial esters derived from fatty acids and hexitol, such as, for example, polyoxyethylene sorbitol monooleate; and condensation products of ethylene oxide with partial esters derived from fatty acids and hexitol anhydrides, such as, for example, polyethylene sorbitan monooleate. An aqueous suspension can also contain at least one preservative, such as, for example, ethyl and n-propyl p-hydroxybenzoate; at least one coloring agent; at least one flavoring agent; and/or at least one sweetening agent, including but not limited to, for example, sucrose, saccharin, and aspartame.

Oily suspensions can, for example, be prepared by suspending at least one compound of Formula (I) in either a vegetable oil, such as, for example, arachis oil; olive oil; sesame oil; and coconut oil; or in mineral oil, such as, for example, liquid paraffin. An oily suspension can also contain at least one thickening agent, such as, for example, beeswax; hard paraffin; and cetyl alcohol. In order to provide a palatable oily suspension, at least one of the sweetening agents already described hereinabove, and/or at least one flavoring agent can be added to the oily suspension. An oily suspension can further contain at least one preservative, including, but not limited to, for example, an antioxidant, such as, for example, butylated hydroxyanisol, and alpha-tocopherol.

Dispersible powders and granules can, for example, be prepared by admixing at least one compound of Formula (I) with at least one dispersing and/or wetting agent; at least one suspending agent; and/or at least one preservative. Suitable dispersing agents, wetting agents, and suspending agents are as already described above. Exemplary preservatives include, but are not limited to, for example, anti-oxidants, e.g., ascorbic acid. In addition, dispersible powders and granules can also contain at least one excipient, including, but not limited to, for example, sweetening agents; flavoring agents; and coloring agents.

An emulsion of at least one compound of Formula (I) can, for example, be prepared as an oil-in-water emulsion. The oily phase of the emulsions comprising compounds of Formula (I) may be constituted from known ingredients in a known manner. The oil phase can be provided by, but is not limited to, for example, a vegetable oil, such as, for example, olive oil and arachis oil; a mineral oil, such as, for example, liquid paraffin; and mixtures thereof. While the phase may comprise merely an emulsifier, it may comprise a mixture of at least one emulsifier with a fat or an oil or with both a fat and an oil. Suitable emulsifying agents include, but are not limited to, for example, naturally-occurring phosphatides, e.g., soy bean lecithin; esters or partial esters derived from fatty acids and hexitol anhydrides, such as, for example, sorbitan monooleate; and condensation products of partial esters with ethylene oxide, such as, for example, polyoxyethylene sorbitan monooleate. Preferably, a hydrophilic emulsifier is included together with a lipophilic emulsifier which acts as a stabilizer. It is also preferred to include both an oil and a fat. Together, the emulsifier(s) with or without stabilizer(s) make-up the so-called emulsifying wax, and the wax together with the oil and fat make up the so-called emulsifying ointment base which forms the oily dispersed phase of the cream formulations. An emulsion can also contain a sweetening agent, a flavoring agent, apreservative, and/or an antioxidant. Emulsifiers and emulsion stabilizers suitable for use in the formulation of the present invention include Tween 60, Span 80, cetostearyl alcohol, myristyl alcohol, glyceryl monostearate, sodium lauryl sulfate, glyceryl distearate alone or with a wax, or other materials well known in the art.

In another embodiment, the compounds of Formula (I) can be formulated as a nanoparticle, lipid nanoparticle, microparticle or liposome.

The compounds of Formula (I) can, for example, also be delivered intravenously, subcutaneously, and/or intramuscularly via any pharmaceutically acceptable and suitable injectable form. Exemplary injectable forms include, but are not limited to, for example, sterile aqueous solutions comprising acceptable vehicles and solvents, such as, for example, water, Ringer's solution, and isotonic sodium chloride solution; sterile oil-in-water microemulsions; and aqueous or oleaginous suspensions. For example, the composition may be provided for intravenous administration comprising an amount of active ingredient in the range of from about 0.2 to 150 mg. In another embodiment, the active ingredient is present in the range of from about 0.3 to 10 mg. In another embodiment, the active ingredient is present in the range of from about 4 to 8.4 mg. In one embodiment, the active ingredient is administered at a dose of about 4 mg. In another embodiment, the active ingredient is administered at a dose of about 6 mg. In another embodiment, the active ingredient is administered at a dose of about 8.4 mg.

In another embodiment, the active ingredient is administered at a dose of about 0.3 mg. In another embodiment, the active ingredient is administered at a dose of about 0.6 mg. In another embodiment, the active ingredient is administered at a dose of about 1.2 mg. In another embodiment, the active ingredient is administered at a dose of about 2.4 mg.

In one embodiment, the composition comprising one or more compounds represented by the structure of Formula (I) or Formula (III) as described herein may be provided for intravenous administration. In one embodiment, the composition for use as described herein comprises 0.2 to 150 mg of one or more compounds represented by the structure of Formula (III), as described herein. In another embodiment, the composition for use as described herein comprises one or more compounds represented by the structure of Formula (III) in the range of from about 0.3 to 10 mg. In another embodiment, one or more compounds represented by the structure of Formula (III) is present in the range of from about 4 to 8.4 mg. In one embodiment, one or more compounds represented by the structure of Formula (III) is administered at a dose of about 2.4 mg. In another embodiment, one or more compounds represented by the structure of Formula (III) is administered at a dose of about 4 mg. In another embodiment, one or more compounds represented by the structure of Formula (III) is administered at a dose of about 6 mg. In another embodiment, one or more compounds represented by the structure of Formula (III) is administered at a dose of about 8.4 mg.

In one embodiment, the first dose comprises 6 mg of Compound (1) or another compound of Formula (III), and the second dose comprises a lower dosage of Compound (1) or another compound of Formula (III), which in one embodiment, comprises 4 mg of Compound (1) or another compound of Formula (III), and, in another embodiment, comprises 2.4 mg of Compound (1) or another compound of Formula (III). In another embodiment, the third does of Compound (1) or another compound of Formula (III) comprises a lower dosage of Compound (1) or another compound of Formula (III), which in one embodiment, comprises 2.4 mg of Compound (1) or another compound of Formula (III). In one embodiment, the dose of Compound (1) or related compounds is lowered if the subject experiences Grade 4 neutropenia lasting ≥7 days, Grade 3 or 4 febrile neutropenia lasting >24 hours, or Grade 4 thrombocytopenia or ≥Grade 3 thrombocytopenia with significant bleeding

Formulations for parenteral administration may be in the form of aqueous or non-aqueous isotonic sterile injection solutions or suspensions. These solutions and suspensions may be prepared from sterile powders or granules using one or more of the carriers or diluents mentioned for use in the formulations for oral administration or by using other suitable dispersing or wetting agents and suspending agents. The compounds may be dissolved in water, polyethylene glycol, propylene glycol, ethanol, corn oil, cottonseed oil, peanut oil, sesame oil, benzyl alcohol, sodium chloride, tragacanth gum, and/or various buffers. Other adjuvants and modes of administration are well and widely known in the pharmaceutical art. The active ingredient may also be administered by injection as a composition with suitable carriers including saline, dextrose, or water, or with cyclodextrin (i.e., CAPTISOL®), cosolvent solubilization (i.e., propylene glycol) or micellar solubilization (i.e., Tween 80).

The sterile injectable preparation may also be a sterile injectable solution or suspension in a non-toxic parenterally acceptable diluent or solvent, for example as a solution in 1,3-butanediol. Among the acceptable vehicles and solvents that may be employed are water, Ringer's solution, and isotonic sodium chloride solution. In addition, sterile, fixed oils are conventionally employed as a solvent or suspending medium. For this purpose, any bland fixed oil may be employed, including synthetic mono- or diglycerides. In addition, fatty acids such as oleic acid find use in the preparation of injectables.

A sterile injectable oil-in-water microemulsion can, for example, be prepared by 1) dissolving at least one compound of Formula (I) in an oily phase, such as, for example, a mixture of soybean oil and lecithin; 2) combining the Formula (I) containing oil phase with a water and glycerol mixture; and 3) processing the combination to form a microemulsion.

A sterile aqueous or oleaginous suspension can be prepared in accordance with methods already known in the art. For example, a sterile aqueous solution or suspension can be prepared with a non-toxic parenterally-acceptable diluent or solvent, such as, for example, 1,3-butane diol; and a sterile oleaginous suspension can be prepared with a sterile non-toxic acceptable solvent or suspending medium, such as, for example, sterile fixed oils, e.g., synthetic mono- or diglycerides; and fatty acids, such as, for example, oleic acid.

Pharmaceutically acceptable carriers, adjuvants, and vehicles that may be used in the pharmaceutical compositions of this invention include, but are not limited to, ion exchangers, alumina, aluminum stearate, lecithin, self-emulsifying drug delivery systems (SEDDS) such as d-alpha-tocopherol polyethyleneglycol 1000 succinate, surfactants used in pharmaceutical dosage forms such as Tweens, polyethoxylated castor oil such as CREMOPHOR® surfactant (BASF), or other similar polymeric delivery matrices, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat. Cyclodextrins such as alpha-, beta-, and gamma-cyclodextrin, or chemically modified derivatives such as hydroxyalkylcyclodextrins, including 2- and 3-hydroxypropyl-cyclodextrins, or other solubilized derivatives may also be advantageously used to enhance delivery of compounds of the formulae described herein.

The pharmaceutically active compounds of this invention can be processed in accordance with conventional methods of pharmacy to produce medicinal agents for administration to patients, including humans and other mammals. The pharmaceutical compositions may be subjected to conventional pharmaceutical operations such as sterilization and/or may contain conventional adjuvants, such as preservatives, stabilizers, wetting agents, emulsifiers, buffers etc. Tablets and pills can additionally be prepared with enteric coatings. Such compositions may also comprise adjuvants, such as wetting, sweetening, flavoring, and perfuming agents.

The amounts of compounds that are administered and the dosage regimen for treating a disease condition with the compounds and/or compositions of this invention depends on a variety of factors, including the age, weight, gender, the medical condition of the subject, the type of disease, the severity of the disease, the route and frequency of administration, and the particular compound employed. Thus, the dosage regimen may vary widely, but can be determined routinely using standard methods. A daily dose of about 0.001 to 100 mg/kg body weight, preferably between about 0.005 and about 50 mg/kg body weight and most preferably between about 0.01 to 10 mg/kg body weight, may be appropriate.

For therapeutic purposes, the active compounds of this invention are ordinarily combined with one or more adjuvants appropriate to the indicated route of administration. If administered orally, the compounds may be admixed with lactose, sucrose, starch powder, cellulose esters of alkanoic acids, cellulose alkyl esters, talc, stearic acid, magnesium stearate, magnesium oxide, sodium and calcium salts of phosphoric and sulfuric acids, gelatin, acacia gum, sodium alginate, polyvinylpyrrolidone, and/or polyvinyl alcohol, and then tableted or encapsulated for convenient administration. Such capsules or tablets may contain a controlled-release formulation as may be provided in a dispersion of active compound in hydroxypropylmethyl cellulose.

Pharmaceutical compositions of this invention comprise at least one compound of Formula (I) and/or at least one salt thereof, and optionally an additional agent selected from any pharmaceutically acceptable carrier, adjuvant, and vehicle. Alternate compositions of this invention comprise a compound of the Formula (I) described herein, or a prodrug thereof, and a pharmaceutically acceptable carrier, adjuvant, or vehicle.

The compound in accordance with Formula (I) can be administered by any means suitable for the condition to be treated, which can depend on the need for site-specific treatment or quantity of Formula (I) compound to be delivered. The compounds and compositions of the present invention may, for example, be administered orally, mucosally, or parentally including intravascularly, intraperitoneally, subcutaneously, intramuscularly, and intrasternally. In one embodiment, the compounds and compositions of the present invention are administered intravenously.

### Methods of Use

In one embodiment, the present invention provides the use of the described compounds or compositions for treating, suppressing or inhibiting a proliferative disease in a subject.

In another embodiment, the present invention provides a method of treating, suppressing or inhibiting a proliferative disease in a subject, comprising the step of administering to said subject a composition comprising one or more compounds of Formula (I): and/or at least one salt thereof,

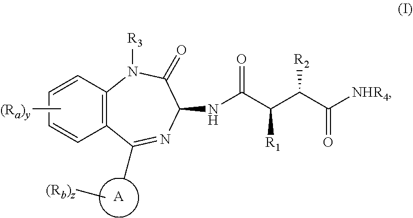

wherein:
R₁ is —CH₂CF₃ or —CH₂CH₂CF₃;
R₂ is —CH₂CF₃, —CH₂CH₂CF₃, or —CH₂CH₂CH₂CF₃;
R₃ is H, —CH₃ or Rₓ;
R₄ is H or R_{y};
Rₓ
is: —CH₂OC(O)CH(CH₃)NH₂, —CH₂OC(O)CH(NH₂)CH(CH₃)₂, —CH₂OC(O)CH((CH(CH₃)₂)NHC(O)CH(NH₂)CH(CH₃)₂,

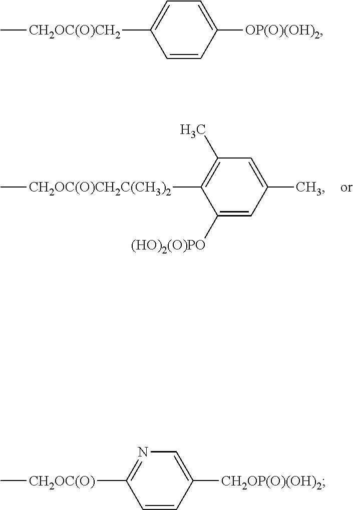

R_{y} is: —SCH₂CH(NH₂)C(O)OH, —SCH₂CH(NH₂)C(O)OH₃,
or —SCH₂CH(NH₂)C(O)OC(CH₃)₃;
Ring A is phenyl or pyridinyl;
each Rₐ is independently F, Cl, —CN, —OCH₃, C₁₋₃ alkyl, —CH₂OH, —CF₃, cyclopropyl, —OCH₃, —O(cyclopropyl) and/or —NHCH₂CH₂OCH₃;
each R_{b} is independently F, Cl, —CH₃, —CH₂OH, —CF₃, cyclopropyl, and/or —OCH₃;
y is zero, 1 or 2; and
z is zero, 1, or 2.

In another embodiment, the present invention provides a method of treating, suppressing or inhibiting a proliferative disease in a subject, comprising the step of administering to said subject a composition comprising one or more compounds of Formula (III):

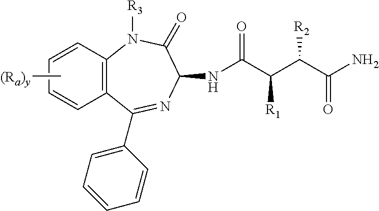

wherein:
R₁ is —CH₂CF₃ or —CH₂CH₂CF₃;
R₂ is —CH₂CF₃, —CH₂CH₂CF₃, or —CH₂CH₂CH₂CF₃;
R₃ is H or —CH₃;
each Rₐ is independently F, Cl, —CN, —OCH₃, and/or —NHCH₂CH₂OCH₃; and
y is zero, 1, or 2.

In one embodiment, the compound is administered at a dose of approximately 0.3, 0.6, 1.2, 2.4, 4, 6, or 8.4 mg.

In one embodiment, the compound is administered intravenously at a dose of approximately 0.3, 0.6, 1.2, 2.4, 4, 6, or 8.4 mg. In another embodiment, the compound is administered weekly at a dose of approximately 0.3, 0.6, 1.2, 2.4, 4, 6, or 8.4 mg.

In another embodiment, the present invention provides a method of treating, suppressing or inhibiting a proliferative disease in a subject comprising the step of administering to said subject a composition comprising one or more compounds represented by the structure of Formula (I) as described hereinabove, wherein said compound is administered at a dose of about 6 mg. In one embodiment, the compound is administered intravenously at a dose of approximately 6 mg. In another embodiment, the compound is administered weekly at a dose of approximately 6 mg.

In another embodiment, the present invention provides a method of treating, suppressing or inhibiting a proliferative disease in a subject comprising the step of administering to said subject a composition consisting essentially of one or more compounds represented by the structure of Formula (I) as described hereinabove. In another embodiment, the present invention provides a method of treating, suppressing or inhibiting a proliferative disease in a subject comprising the step of administering to said subject a composition consisting of one or more compounds represented by the structure of Formula (I) as described hereinabove.

In one embodiment, the present invention provides the use of a therapeutically acceptable amount of one or more compounds or compositions as described herein for treating, suppressing or inhibiting a proliferative disease in a subject. In another embodiment, the present invention provides the use of a therapeutically effective amount of one or more compounds or compositions as described herein for treating, suppressing or inhibiting a proliferative disease in a subject. In another embodiment, the present invention provides the use of a synergistically effective amount of one or more compounds or compositions as described herein for treating, suppressing or inhibiting a proliferative disease in a subject. In another embodiment, the present invention provides the use of a synergistically therapeutically effective amount of one or more compounds or compositions as described herein for treating, suppressing or inhibiting a proliferative disease in a subject.

In one embodiment, the proliferative disease comprises a Desmoid tumor.

In one embodiment, the proliferative disease comprises a pre-cancerous condition or a benign proliferative disorder.

In one embodiment, the term “pre-cancerous” or, alternatively, “pre-malignant” as used herein interchangeably refers to diseases, syndromes or other conditions associated with an increased risk of cancer. Pre-cancerous conditions in the context of the present invention include, but are not limited to: breast calcifications, vaginal intra-epithelial neoplasia, Barrett's esophagus, atrophic gastritis, dyskeratosis congenital, sideropenic dysphagia, lichen planus, oral submucous fibrosis, actinic keratosis, solar elastosis, cervical dysplasia, leukoplakia and erythroplakia.

In one embodiment, the term “benign hyperproliferative disorder” as used herein refers to a condition in which there is an abnormal growth and differentiation of cells and an increase in the amount of organic tissue that results from cell proliferation. The benign hyperproliferative disorder may be attributed to lack of response or inappropriate response to regulating factors, or alternatively to dysfunctional regulating factors. Non-limiting examples of benign hyperproliferative disorder are psoriasis and benign prostatic hyperplasia (BPH).

In another embodiment, the proliferative disease comprises a cancer.

In one embodiment, the cancer comprises a solid tumor. In another embodiment, the cancer comprises a hematological malignancy.

In one embodiment, a subject as described herein has cancer. In one embodiment, the term “cancer” in the context of the present invention includes all types of neoplasm whether in the form of solid or non-solid tumors and includes both malignant and premalignant conditions as well as their metastasis.

In one embodiment, the cancer is a carcinoma, sarcoma, myeloma, leukemia, or lymphoma. In another embodiment, the cancer is a mixed type.

In one embodiment, mixed type cancers comprise several types of cells. The type components may be within one category or from different categories. Some examples are: adenosquamous carcinoma; mixed mesodermal tumor; carcinosarcoma; teratocarcinoma

In another embodiment, the carcinoma comprises Adenoid Cystic Carcinoma (ACC).

In another embodiment, the carcinoma comprises Gastro-esophageal junction carcinoma.

In one embodiment, the carcinoma is an adenocarcinoma. In another embodiment, the carcinoma is a squamous cell carcinoma.

In one embodiment, the sarcoma comprises osteosarcoma or osteogenic sarcoma (bone); Chondrosarcoma (cartilage); Leiomyosarcoma (smooth muscle); Rhabdomyosarcoma (skeletal muscle); Mesothelial sarcoma or mesothelioma (membranous lining of body cavities); Fibrosarcoma (fibrous tissue); Angiosarcoma or hemangioendothelioma (blood vessels); Liposarcoma (adipose tissue); Glioma or astrocytoma (neurogenic connective tissue found in the brain); Myxosarcoma (primitive embryonic connective tissue); and Mesenchymous or mixed mesodermal tumor (mixed connective tissue types).

In one embodiment, the cancer comprises myeloma, which, in one embodiment, is cancer that originates in the plasma cells of bone marrow. The plasma cells produce some of the proteins found in blood. In one embodiment, the cancer comprises multiple myeloma.

In another embodiment, the cancer comprises leukemia (“non-solid tumor” or “blood cancer”), which in one embodiment, is a cancer of the bone marrow (the site of blood cell production). In one embodiment, leukemia comprises myelogenous or granulocytic leukemia (malignancy of the myeloid and granulocytic white blood cell series); Lymphatic, lymphocytic, or lymphoblastic leukemia (malignancy of the lymphoid and lymphocytic blood cell series); and Polycythemia vera or erythremia (malignancy of various blood cell products, but with red cells predominating).

In another embodiment, the cancer comprises T-cell acute lymphoblastic leukemia (T-ALL). In another embodiment, the cancer comprises T-lymphoblastic leukemia/lymphoma (TLL). In another embodiment, the cancer comprises Chronic Lymphocytic Leukemia (CLL).

In another embodiment, the cancer comprises a lymphoma. In one embodiment, the lymphoma comprises an extranodal lymphoma. In one embodiment, the lymphoma comprises a Hodgkin lymphoma. In another embodiment, the lymphoma comprises a Non-Hodgkin lymphoma.

In one embodiment, the lymphoma comprises a marginal zone B cell lymphoma, a diffuse large B cell lymphoma, or a mantle cell lymphoma.

In another embodiment, the cancer is dependent upon Notch activation. In another embodiment, the cancer comprises a Notch-activating genetic alteration. In another embodiment, the cancer comprises a Notch-activated IHC stain. In another embodiment, the cancer comprises a Notch-active gene expression profile.

In one embodiment, the present invention provides a method of treating cancer, wherein said cancer comprises one or more Notch-activating genetic alterations, comprising the step of administering to said subject a composition comprising one or more compounds represented by the structure of Formula (I): and/or at least one salt thereof,

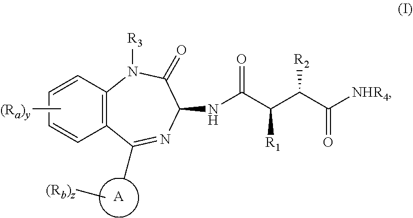

wherein:
R₁ is —CH₂CF₃ or —CH₂CH₂CF₃;
R₂ is —CH₂CF₃, —CH₂CH₂CF₃, or —CH₂CH₂CH₂CF₃;
R₃ is H, —CH₃ or Rₓ;
R₄ is H or R_{y};
Rₓ
is: —CH₂OC(O)CH(CH₃)NH₂, —CH₂OC(O)CH(NH₂)CH(CH₃)₂, —CH₂OC(O)CH((CH(CH₃)₂)NHC(O)CH(NH₂)CH(CH₃)₂,

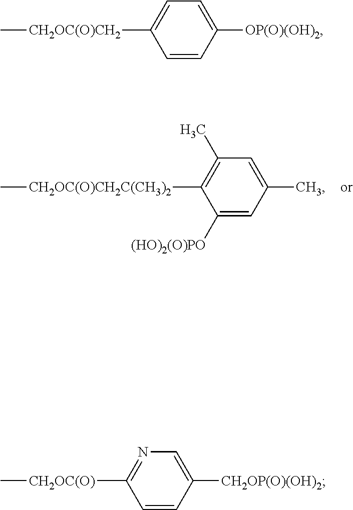

R_{y} is: —SCH₂CH(NH₂)C(O)OH, —SCH₂CH(NH₂)C(O)OH₃,
or —SCH₂CH(NH₂)C(O)OC(CH₃)₃;
Ring A is phenyl or pyridinyl;
each Rₐ is independently F, Cl, —CN, —OCH₃, C₁₋₃ alkyl, —CH₂OH, —CF₃, cyclopropyl, —OCH₃, —O(cyclopropyl) and/or —NHCH₂CH₂OCH₃;
each R_{b} is independently F, Cl, —CH₃, —CH₂OH, —CF₃, cyclopropyl, and/or —OCH₃;
y is zero, 1 or 2; and
z is zero, 1, or 2.

In another embodiment, the present invention provides a method of treating cancer, wherein said cancer comprises one or more Notch-activating genetic alterations, comprising the step of administering to said subject a composition comprising one or more compounds represented by the structure of Formula (III):

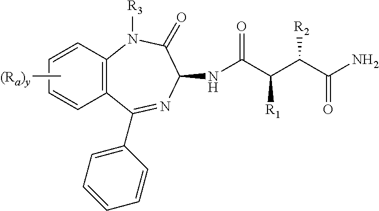

or prodrugs or salts thereof; wherein:
R₁ is —CH₂CF₃ or —CH₂CH₂CF₃;
R₂ is —CH₂CF₃, —CH₂CH₂CF₃, or —CH₂CH₂CH₂CF₃;
R₃ is H or —CH₃;
each Rₐ is independently F, Cl, —CN, —OCH₃, and/or —NHCH₂CH₂OCH₃; and
y is zero, 1, or 2.

In another embodiment, the present invention provides a method of treating cancer, wherein said cancer comprises one or more Notch-activating genetic alterations, comprising the step of administering to said subject a composition comprising:

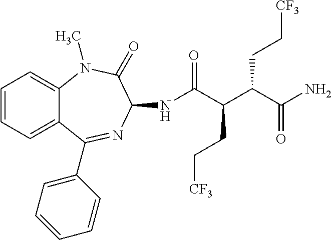 [SMILES: CN1C(=O)[C@@H](NC(=O)[C@H](CCC(F)(F)F)[C@H](CCC(F)(F)F)C(N)=O)N=C(c2ccccc2)c2ccccc21]

In another embodiment, the present invention provides a method of treating cancer, wherein said cancer comprises one or more Notch-activating genetic alterations, comprising the step of administering to said subject a composition comprising:

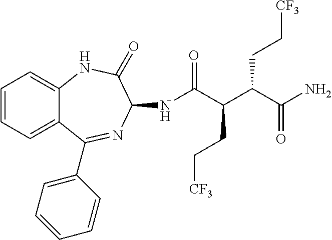 [SMILES: NC(=O)[C@@H](CCC(F)(F)F)[C@@H](CCC(F)(F)F)C(=O)N[C@H]1N=C(c2ccccc2)c2ccccc2NC1=O]

In one embodiment, the present invention provides a method of treating breast cancer, wherein said breast cancer comprises one or more Notch-activating genetic alterations, comprising the step of administering to said subject a composition comprising one or more compounds represented by the structure of Formula (I): and/or at least one salt thereof,

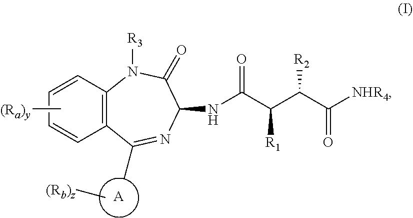

wherein:
R₁ is —CH₂CF₃ or —CH₂CH₂CF₃;
R₂ is —CH₂CF₃, —CH₂CH₂CF₃, or —CH₂CH₂CH₂CF₃;
R₃ is H, —CH₃ or Rₓ;
R₄ is H or R_{y};
Rₓ
is: —CH₂OC(O)CH(CH₃)NH₂, —CH₂OC(O)CH(NH₂)CH(CH₃)₂, —CH₂OC(O)CH((CH(CH₃)₂)NHC(O)CH(NH₂)CH(CH₃)₂,

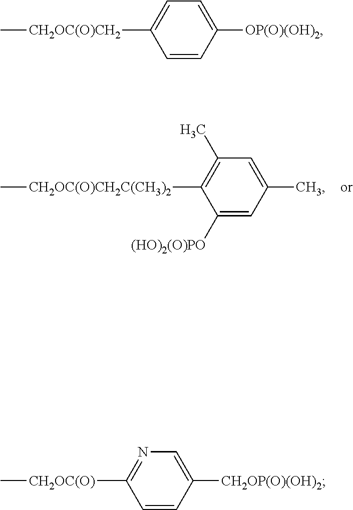

R_{y} is: —SCH₂CH(NH₂)C(O)OH, —SCH₂CH(NH₂)C(O)OH₃,
or —SCH₂CH(NH₂)C(O)OC(CH₃)₃;
Ring A is phenyl or pyridinyl;
each Rₐ is independently F, Cl, —CN, —OCH₃, C₁₋₃ alkyl, —CH₂OH, —CF₃, cyclopropyl, —OCH₃, —O(cyclopropyl) and/or —NHCH₂CH₂OCH₃;
each R_{b} is independently F, Cl, —CH₃, —CH₂OH, —CF₃, cyclopropyl, and/or —OCH₃;
y is zero, 1 or 2; and
z is zero, 1, or 2.

In another embodiment, the present invention provides a method of treating breast cancer, wherein said breast cancer comprises one or more Notch-activating genetic alterations, comprising the step of administering to said subject a composition comprising one or more compounds represented by the structure of Formula (III):

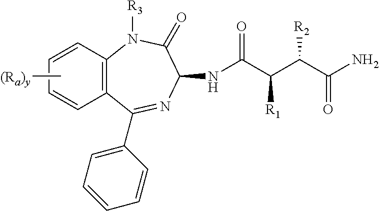

or prodrugs or salts thereof; wherein:
R₁ is —CH₂CF₃ or —CH₂CH₂CF₃;
R₂ is —CH₂CF₃, —CH₂CH₂CF₃, or —CH₂CH₂CH₂CF₃;
R₃ is H or —CH₃;
each Rₐ is independently F, Cl, —CN, —OCH₃, and/or —NHCH₂CH₂OCH₃; and
y is zero, 1, or 2.

In another embodiment, the present invention provides a method of treating breast cancer, wherein said breast cancer comprises one or more Notch-activating genetic alterations, comprising the step of administering to said subject a composition comprising:

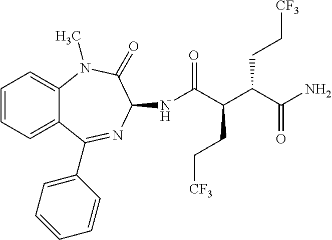 [SMILES: CN1C(=O)[C@@H](NC(=O)[C@H](CCC(F)(F)F)[C@H](CCC(F)(F)F)C(N)=O)N=C(c2ccccc2)c2ccccc21]

In another embodiment, the present invention provides a method of treating breast cancer, wherein said breast cancer comprises one or more Notch-activating genetic alterations, comprising the step of administering to said subject a composition comprising:

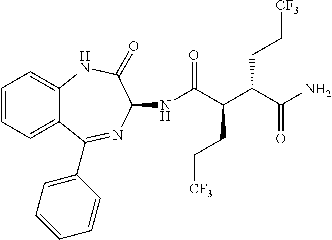 [SMILES: NC(=O)[C@@H](CCC(F)(F)F)[C@@H](CCC(F)(F)F)C(=O)N[C@H]1N=C(c2ccccc2)c2ccccc2NC1=O]

In one embodiment, the breast cancer comprises triple-negative breast cancer (TNBC). In one embodiment, triple-negative breast cancer cells lack receptors for estrogen (ER), progesterone (PR) or HER2. In one embodiment, hormone therapies or medications that work by blocking HER2, such as trastuzumab, are not effective in treating breast cancer that is ER, PR and HER2 negative. In one embodiment, TNBC constitutes 10%-20% of all breast cancers. In one embodiment, TNBC tumors are larger in size as compared with other breast cancer tumors. In another embodiment, cells from TNBC tumors have less differentiated cell morphology compared with cells from other breast cancer tumors. In another embodiment, TNBC tumors have more lymph node involvement at diagnosis as compared with other breast cancer tumors. In another embodiment, TNBC tumors are biologically more aggressive as compared with other breast cancer tumors. In one embodiment, TNBC patients have shorter post-relapse survival as compared with other breast cancer patients. In another embodiment, TNBC patients have poor overall survival rates as compared with other breast cancer patients.

In one embodiment, the present invention provides a method of treating TNBC, wherein said TNBC comprises one or more Notch-activating genetic alterations, comprising the step of administering to said subject a composition comprising one or more compounds represented by the structure of Formula (I): and/or at least one salt thereof,

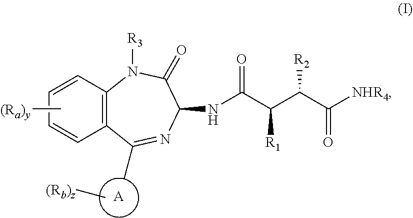

wherein:
R₁ is —CH₂CF₃ or —CH₂CH₂CF₃;
R₂ is —CH₂CF₃, —CH₂CH₂CF₃, or —CH₂CH₂CH₂CF₃;
R₃ is H, —CH₃ or Rₓ;
R₄ is H or R_{y};
Rₓ is: —CH₂OC(O)CH(CH₃)NH₂, —CH₂OC(O)CH(NH₂)CH(CH₃)₂, —CH₂OC(O)CH((CH(CH₃)₂)NHC(O)CH(NH₂)CH(CH₃)₂,

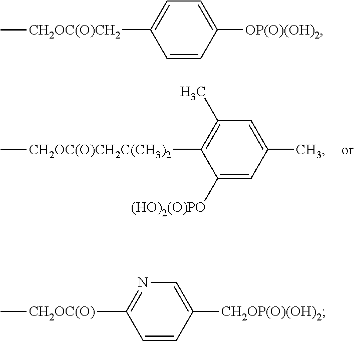

R_{y} is: —SCH₂CH(NH₂)C(O)OH, —SCH₂CH(NH₂)C(O)OH₃,
or —SCH₂CH(NH₂)C(O)OC(CH₃)₃;
Ring A is phenyl or pyridinyl;
each Rₐ is independently F, Cl, —CN, —OCH₃, C₁₋₃ alkyl, —CH₂OH, —CF₃, cyclopropyl, —OCH₃, —O(cyclopropyl) and/or —NHCH₂CH₂OCH₃;
each R_{b} is independently F, Cl, —CH₃, —CH₂OH, —CF₃, cyclopropyl, and/or —OCH₃;
y is zero, 1 or 2; and
z is zero, 1, or 2.

In another embodiment, the present invention provides a method of treating TNBC, wherein said TNBC comprises one or more Notch-activating genetic alterations, comprising the step of administering to said subject a composition comprising one or more compounds represented by the structure of Formula (III):

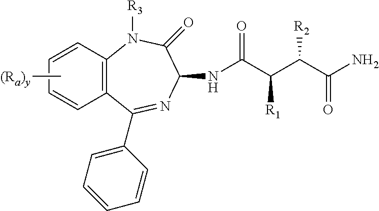

or prodrugs or salts thereof; wherein:
R₁ is —CH₂CF₃ or —CH₂CH₂CF₃;
R₂ is —CH₂CF₃, —CH₂CH₂CF₃, or —CH₂CH₂CH₂CF₃;
R₃ is H or —CH₃;
each Rₐ is independently F, Cl, —CN, —OCH₃, and/or —NHCH₂CH₂OCH₃; and
y is zero, 1, or 2.

In another embodiment, the present invention provides a method of treating TNBC, wherein said TNBC comprises one or more Notch-activating genetic alterations, comprising the step of administering to said subject a composition comprising:

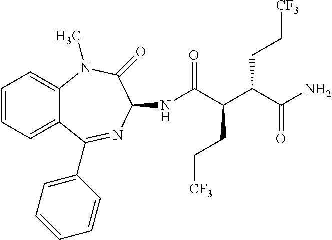 [SMILES: CN1C(=O)[C@@H](NC(=O)[C@H](CCC(F)(F)F)[C@H](CCC(F)(F)F)C(N)=O)N=C(c2ccccc2)c2ccccc21]

In another embodiment, the present invention provides a method of treating TNBC, wherein said TNBC comprises one or more Notch-activating genetic alterations, comprising the step of administering to said subject a composition comprising:

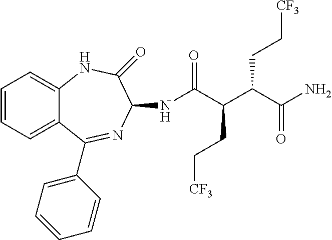 [SMILES: NC(=O)[C@@H](CCC(F)(F)F)[C@@H](CCC(F)(F)F)C(=O)N[C@H]1N=C(c2ccccc2)c2ccccc2NC1=O]

In one embodiment, the present invention provides a method of reducing tumor size in a subject having cancer, wherein said cancer comprises one or more Notch-activating genetic alterations, comprising the step of administering to said subject a composition comprising one or more compounds represented by the structure of Formula (I): and/or at least one salt thereof, as described herein.

In another embodiment, the present invention provides a method of reducing tumor size in a subject having a tumor characterized by an activated Notch pathway, comprising the steps as described herein. In another embodiment, one or more cells of the tumor comprise one or more Notch-activating genetic alterations and/or overexpression of one or more Notch-regulated genes and/or an activated Notch IHC stain.

In one embodiment, the present invention provides a method of reducing tumor size in a subject having breast cancer, wherein said breast cancer comprises one or more Notch-activating genetic alterations, comprising the step of administering to said subject a composition comprising one or more compounds represented by the structure of Formula (I): and/or at least one salt thereof, as described herein.

In one embodiment, the present invention provides a method of reducing tumor size in a subject having triple-negative breast cancer (TNBC), wherein one or more cells of the TNBC tumor comprises one or more Notch-activating genetic alterations, comprising the step of administering to said subject a composition comprising one or more compounds represented by the structure of Formula (I): and/or at least one salt thereof,

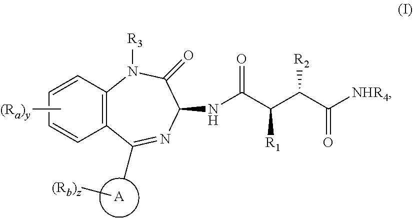

wherein:
R₁ is —CH₂CF₃ or —CH₂CH₂CF₃;
R₂ is —CH₂CF₃, —CH₂CH₂CF₃, or —CH₂CH₂CH₂CF₃;
R₃ is H, —CH₃ or Rₓ;
R₄ is H or R_{y};
Rₓ
is: —CH₂OC(O)CH(CH₃)NH₂, —CH₂OC(O)CH(NH₂)CH(CH₃)₂, —CH₂OC(O)CH((CH(CH₃)₂)NHC(O)CH(NH₂)CH(CH₃)₂,

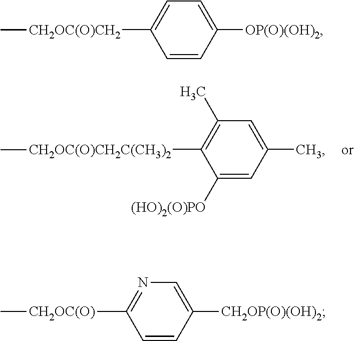

R_{y} is: —SCH₂CH(NH₂)C(O)OH, —SCH₂CH(NH₂)C(O)OH₃,
or —SCH₂CH(NH₂)C(O)OC(CH₃)₃;
Ring A is phenyl or pyridinyl;
each Rₐ is independently F, Cl, —CN, —OCH₃, C₁₋₃ alkyl, —CH₂OH, —CF₃, cyclopropyl, —OCH₃, —O(cyclopropyl) and/or —NHCH₂CH₂OCH₃;
each R_{b} is independently F, Cl, —CH₃, —CH₂OH, —CF₃, cyclopropyl, and/or —OCH₃;
y is zero, 1 or 2; and
z is zero, 1, or 2.

In another embodiment, the present invention provides a method of reducing tumor size in a subject having triple-negative breast cancer (TNBC), wherein one or more cells of the TNBC tumor comprises one or more Notch-activating genetic alterations, comprising the step of administering to said subject a composition comprising one or more compounds represented by the structure of Formula (III):

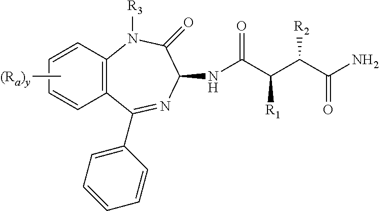

or prodrugs or salts thereof; wherein:
R₁ is —CH₂CF₃ or —CH₂CH₂CF₃;
R₂ is —CH₂CF₃, —CH₂CH₂CF₃, or —CH₂CH₂CH₂CF₃;
R₃ is H or —CH₃;
each Rₐ is independently F, Cl, —CN, —OCH₃, and/or —NHCH₂CH₂OCH₃; and
y is zero, 1, or 2.

In another embodiment, the present invention provides a method of reducing tumor size in a subject having triple-negative breast cancer (TNBC), wherein one or more cells of said TNBC comprises one or more Notch-activating genetic alterations, comprising the step of administering to said subject a composition comprising:

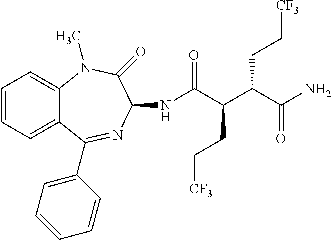 [SMILES: CN1C(=O)[C@@H](NC(=O)[C@H](CCC(F)(F)F)[C@H](CCC(F)(F)F)C(N)=O)N=C(c2ccccc2)c2ccccc21]

In another embodiment, the present invention provides a method of reducing tumor size in a subject having triple-negative breast cancer (TNBC), wherein one or more cells of said TNBC comprises one or more Notch-activating genetic alterations, comprising the step of administering to said subject a composition comprising:

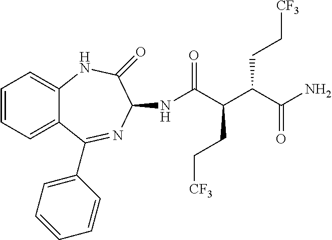 [SMILES: NC(=O)[C@@H](CCC(F)(F)F)[C@@H](CCC(F)(F)F)C(=O)N[C@H]1N=C(c2ccccc2)c2ccccc2NC1=O]

In one embodiment, reducing tumor size comprises decreasing tumor size by 25%-95%. In another embodiment, reducing tumor size comprises decreasing tumor size by 25%. In another embodiment, reducing tumor size comprises decreasing tumor size by 30%. In another embodiment, reducing tumor size comprises decreasing tumor size by 35%. In another embodiment, reducing tumor size comprises decreasing tumor size by 40%. In another embodiment, reducing tumor size comprises decreasing tumor size by 45%. In another embodiment, reducing tumor size comprises decreasing tumor size by 50%. In another embodiment, reducing tumor size comprises decreasing tumor size by 55%. In another embodiment, reducing tumor size comprises decreasing tumor size by 60%. In another embodiment, reducing tumor size comprises decreasing tumor size by 65%. In another embodiment, reducing tumor size comprises decreasing tumor size by 70%. In another embodiment, reducing tumor size comprises decreasing tumor size by 75%. In another embodiment, reducing tumor size comprises decreasing tumor size by 80%. In another embodiment, reducing tumor size comprises decreasing tumor size by 85%. In another embodiment, reducing tumor size comprises decreasing tumor size by 90%. In another embodiment, reducing tumor size comprises decreasing tumor size by 95%.

In one embodiment, the present invention provides a method of reducing tumor volume in a subject having cancer, wherein said cancer comprises one or more Notch-activating genetic alterations, comprising the step of administering to said subject a composition comprising one or more compounds represented by the structure of Formula (I): and/or at least one salt thereof, as described herein.

In one embodiment, the present invention provides a method of reducing tumor volume in a subject having breast cancer, wherein said breast cancer comprises one or more Notch-activating genetic alterations, comprising the step of administering to said subject a composition comprising one or more compounds represented by the structure of Formula (I): and/or at least one salt thereof, as described herein.

In one embodiment, the present invention provides a method of reducing tumor volume in a subject having triple-negative breast cancer (TNBC), wherein one or more cells of the TNBC tumor comprises one or more Notch-activating genetic alterations, comprising the step of administering to said subject a composition comprising one or more compounds represented by the structure of Formula (I): and/or at least one salt thereof,

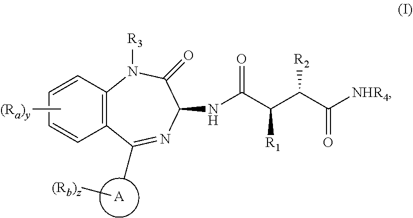

wherein:
R₁ is —CH₂CF₃ or —CH₂CH₂CF₃;
R₂ is —CH₂CF₃, —CH₂CH₂CF₃, or —CH₂CH₂CH₂CF₃;
R₃ is H, —CH₃ or Rₓ;
R₄ is H or R_{y};
Rₓ
is: —CH₂OC(O)CH(CH₃)NH₂, —CH₂OC(O)CH(NH₂)CH(CH₃)₂, —CH₂OC(O)CH((CH(CH₃)₂)NHC(O)CH(NH₂)CH(CH₃)₂,

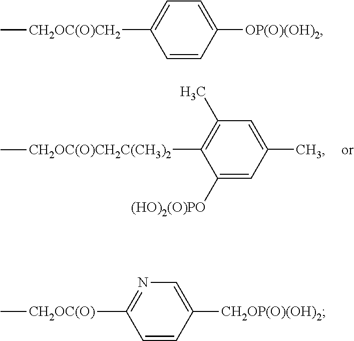

R_{y} is: —SCH₂CH(NH₂)C(O)OH, —SCH₂CH(NH₂)C(O)OH₃,
or —SCH₂CH(NH₂)C(O)OC(CH₃)₃;
Ring A is phenyl or pyridinyl;
each Rₐ is independently F, Cl, —CN, —OCH₃, C₁₋₃ alkyl, —CH₂OH, —CF₃, cyclopropyl, —OCH₃, —O(cyclopropyl) and/or —NHCH₂CH₂OCH₃;
each R_{b} is independently F, Cl, —CH₃, —CH₂OH, —CF₃, cyclopropyl, and/or —OCH₃;
y is zero, 1 or 2; and
z is zero, 1, or 2.

In another embodiment, the present invention provides a method of reducing tumor volume in a subject having triple-negative breast cancer (TNBC), wherein one or more cells of the TNBC tumor comprises one or more Notch-activating genetic alterations, comprising the step of administering to said subject a composition comprising one or more compounds represented by the structure of Formula (III):

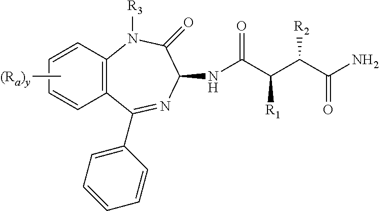

or prodrugs or salts thereof; wherein:
R₁ is —CH₂CF₃ or —CH₂CH₂CF₃;
R₂ is —CH₂CF₃, —CH₂CH₂CF₃, or —CH₂CH₂CH₂CF₃;
R₃ is H or —CH₃;
each Rₐ is independently F, Cl, —CN, —OCH₃, and/or —NHCH₂CH₂OCH₃; and
y is zero, 1, or 2.

In another embodiment, the present invention provides a method of reducing tumor volume in a subject having triple-negative breast cancer (TNBC), wherein one or more cells of said TNBC comprises one or more Notch-activating genetic alterations, comprising the step of administering to said subject a composition comprising:

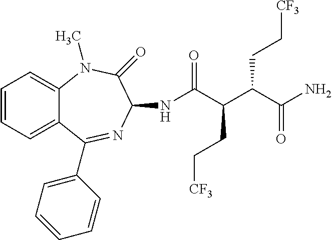 [SMILES: CN1C(=O)[C@@H](NC(=O)[C@H](CCC(F)(F)F)[C@H](CCC(F)(F)F)C(N)=O)N=C(c2ccccc2)c2ccccc21]

In another embodiment, the present invention provides a method of reducing tumor volume in a subject having triple-negative breast cancer (TNBC), wherein one or more cells of said TNBC comprises one or more Notch-activating genetic alterations, comprising the step of administering to said subject a composition comprising:

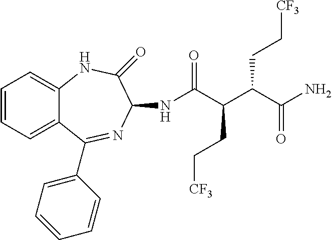 [SMILES: NC(=O)[C@@H](CCC(F)(F)F)[C@@H](CCC(F)(F)F)C(=O)N[C@H]1N=C(c2ccccc2)c2ccccc2NC1=O]

In one embodiment, reducing tumor volume comprises decreasing tumor volume by 25%-95%. In another embodiment, reducing tumor volume comprises decreasing tumor volume by 25%. In another embodiment, reducing tumor volume comprises decreasing tumor volume by 30%. In another embodiment, reducing tumor volume comprises decreasing tumor volume by 35%. In another embodiment, reducing tumor volume comprises decreasing tumor volume by 40%. In another embodiment, reducing tumor volume comprises decreasing tumor volume by 45%. In another embodiment, reducing tumor volume comprises decreasing tumor volume by 50%. In another embodiment, reducing tumor volume comprises decreasing tumor volume by 55%. In another embodiment, reducing tumor volume comprises decreasing tumor volume by 60%. In another embodiment, reducing tumor volume comprises decreasing tumor volume by 65%. In another embodiment, reducing tumor volume comprises decreasing tumor volume by 70%. In another embodiment, reducing tumor volume comprises decreasing tumor volume by 75%. In another embodiment, reducing tumor volume comprises decreasing tumor volume by 80%. In another embodiment, reducing tumor volume comprises decreasing tumor volume by 85%. In another embodiment, reducing tumor volume comprises decreasing tumor volume by 90%. In another embodiment, reducing tumor volume comprises decreasing tumor volume by 95%.

In one embodiment, the present invention provides a method of suppressing tumor growth in a subject having a tumor, wherein one or more cells of said tumor comprises one or more Notch-activating genetic alterations, comprising the step of administering to said subject a composition comprising one or more compounds represented by the structure of Formula (I): and/or at least one salt thereof, as described herein.

In another embodiment, the present invention provides a method of suppressing tumor growth in a subject having a tumor characterized by an activated Notch pathway, comprising the steps as described herein. In another embodiment, one or more cells of the tumor comprise one or more Notch-activating genetic alterations and/or overexpression of one or more Notch-regulated genes. Or presence of Notch activated IHC stain.

In one embodiment, the present invention provides a method of suppressing tumor growth in a subject having breast cancer, wherein one or more cells of said tumor comprises one or more Notch-activating genetic alterations, comprising the step of administering to said subject a composition comprising one or more compounds represented by the structure of Formula (I): and/or at least one salt thereof, as described herein.

In one embodiment, the present invention provides a method of suppressing tumor growth in a subject having triple-negative breast cancer (TNBC), wherein one or more cells of said TNBC comprises one or more Notch-activating genetic alterations, comprising the step of administering to said subject a composition comprising one or more compounds represented by the structure of Formula (I): and/or at least one salt thereof,

In one embodiment, the present invention provides a method of suppressing tumor growth in a subject having triple-negative breast cancer (TNBC) characterized by an activated Notch pathway, comprising the step of administering to said subject a composition comprising one or more compounds represented by the structure of Formula (I): and/or at least one salt thereof,

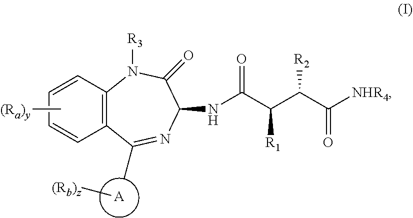

wherein:
R₁ is —CH₂CF₃ or —CH₂CH₂CF₃;
R₂ is —CH₂CF₃, —CH₂CH₂CF₃, or —CH₂CH₂CH₂CF₃;
R₃ is H, —CH₃ or Rₓ;
R₄ is H or R_{y};
Rₓ
is: —CH₂OC(O)CH(CH₃)NH₂, —CH₂OC(O)CH(NH₂)CH(CH₃)₂, —CH₂OC(O)CH((CH(CH₃)₂)NHC(O)CH(NH₂)CH(CH₃)₂,

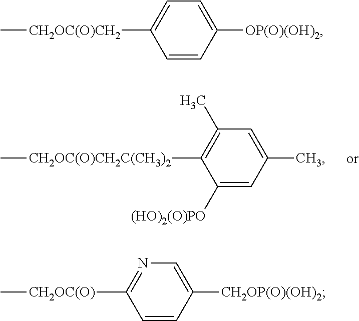

R_{y} is: —SCH₂CH(NH₂)C(O)OH, —SCH₂CH(NH₂)C(O)OH₃,
or —SCH₂CH(NH₂)C(O)OC(CH₃)₃;
Ring A is phenyl or pyridinyl;
each Rₐ is independently F, Cl, —CN, —OCH₃, C₁₋₃ alkyl, —CH₂OH, —CF₃, cyclopropyl, —OCH₃, —O(cyclopropyl) and/or —NHCH₂CH₂OCH₃;
each R_{b} is independently F, Cl, —CH₃, —CH₂OH, —CF₃, cyclopropyl, and/or —OCH₃;
y is zero, 1 or 2; and
z is zero, 1, or 2.

In another embodiment, the present invention provides a method of suppressing tumor growth in a subject having triple-negative breast cancer (TNBC), wherein one or more cells of the TNBC tumor comprises one or more Notch-activating genetic alterations, comprising the step of administering to said subject a composition comprising one or more compounds represented by the structure of Formula (III):

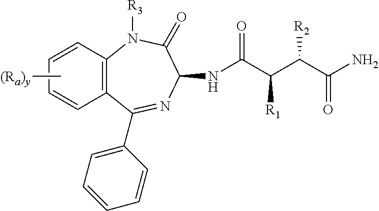

or prodrugs or salts thereof; wherein:
R₁ is —CH₂CF₃ or —CH₂CH₂CF₃;
R₂ is —CH₂CF₃, —CH₂CH₂CF₃, or —CH₂CH₂CH₂CF₃;
R₃ is H or —CH₃;
each Rₐ is independently F, Cl, —CN, —OCH₃, and/or —NHCH₂CH₂OCH₃; and
y is zero, 1, or 2.

In another embodiment, the present invention provides a method of suppressing tumor growth in a subject having triple-negative breast cancer (TNBC), wherein one or more cells of the TNBC tumor comprises one or more Notch-activating genetic alterations, comprising the step of administering to said subject a composition comprising:

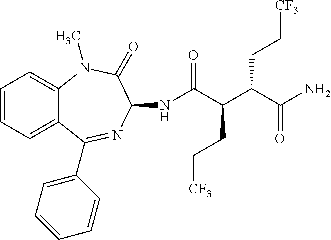 [SMILES: CN1C(=O)[C@@H](NC(=O)[C@H](CCC(F)(F)F)[C@H](CCC(F)(F)F)C(N)=O)N=C(c2ccccc2)c2ccccc21]

In another embodiment, the present invention provides a method of suppressing tumor growth in a subject having triple-negative breast cancer (TNBC), wherein one or more cells of the TNBC tumor comprises one or more Notch-activating genetic alterations, comprising the step of administering to said subject a composition comprising:

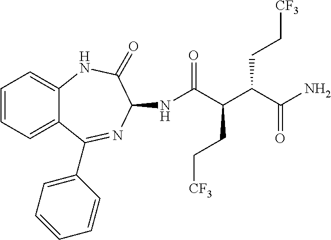 [SMILES: NC(=O)[C@@H](CCC(F)(F)F)[C@@H](CCC(F)(F)F)C(=O)N[C@H]1N=C(c2ccccc2)c2ccccc2NC1=O]

In one embodiment, administration of a composition as described herein suppresses tumor growth by 20-99% compared to untreated tumors, compared to vehicle-treated tumors, compared to placebo-treated tumors, or compared to tumors treated with another anti-cancer therapy. In another embodiment, tumor growth is suppressed by 20-35%. In another embodiment, tumor growth is suppressed by 35-50%. In another embodiment, tumor growth is suppressed by 50-75%. In another embodiment, tumor growth is suppressed by 75-90%. In another embodiment, tumor growth is suppressed by 90-99%.

In another embodiment, tumor growth is suppressed by 20%. In another embodiment, tumor growth is suppressed by 25%. In another embodiment, tumor growth is suppressed by 30%. In another embodiment, tumor growth is suppressed by 35%. In another embodiment, tumor growth is suppressed by 40%. In another embodiment, tumor growth is suppressed by 45%. In another embodiment, tumor growth is suppressed by 50%. In another embodiment, tumor growth is suppressed by 55%. In another embodiment, tumor growth is suppressed by 60%. In another embodiment, tumor growth is suppressed by 65%. In another embodiment, tumor growth is suppressed by 70%. In another embodiment, tumor growth is suppressed by 75%. In another embodiment, tumor growth is suppressed by 80%. In another embodiment, tumor growth is suppressed by 85%. In another embodiment, tumor growth is suppressed by 90%. In another embodiment, tumor growth is suppressed by 95%. In another embodiment, tumor growth is suppressed by 99%.

In one embodiment, the present invention provides a method of inhibiting tumor growth in a subject having a tumor, wherein one or more cells of said tumor comprises one or more Notch-activating genetic alterations, comprising the step of administering to said subject a composition comprising one or more compounds represented by the structure of Formula (I): and/or at least one salt thereof, as described herein.

In another embodiment, the present invention provides a method of inhibiting tumor growth in a subject having a tumor characterized by an activated Notch pathway, comprising the steps as described herein. In another embodiment, one or more cells of the tumor comprise one or more Notch-activating genetic alterations and/or overexpression of one or more Notch-regulated genes.

In one embodiment, the present invention provides a method of inhibiting tumor growth in a subject having breast cancer, wherein one or more cells of said tumor comprises one or more Notch-activating genetic alterations, comprising the step of administering to said subject a composition comprising one or more compounds represented by the structure of Formula (I): and/or at least one salt thereof, as described herein.

In one embodiment, the present invention provides a method of inhibiting tumor growth in a subject having triple-negative breast cancer (TNBC), wherein one or more cells of the TNBC tumor comprises one or more Notch-activating genetic alterations, comprising the step of administering to said subject a composition comprising one or more compounds represented by the structure of Formula (I): and/or at least one salt thereof,

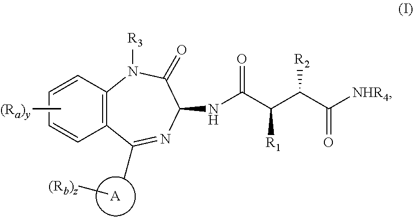

wherein:
R₁ is —CH₂CF₃ or —CH₂CH₂CF₃;
R₂ is —CH₂CF₃, —CH₂CH₂CF₃, or —CH₂CH₂CH₂CF₃;
R₃ is H, —CH₃ or Rₓ;
R₄ is H or R_{y};
Rₓ
is: —CH₂OC(O)CH(CH₃)NH₂, —CH₂OC(O)CH(NH₂)CH(CH₃)₂, —CH₂OC(O)CH((CH(CH₃)₂)NHC(O)CH(NH₂)CH(CH₃)₂,

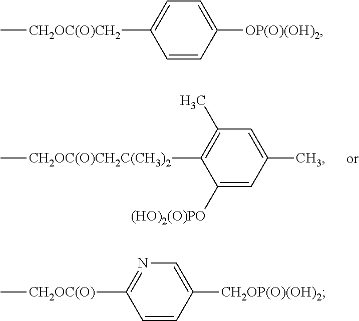

R_{y} is: —SCH₂CH(NH₂)C(O)OH, —SCH₂CH(NH₂)C(O)OH₃,
or —SCH₂CH(NH₂)C(O)OC(CH₃)₃;
Ring A is phenyl or pyridinyl;
each Rₐ is independently F, Cl, —CN, —OCH₃, C₁₋₃ alkyl, —CH₂OH, —CF₃, cyclopropyl, —OCH₃, —O(cyclopropyl) and/or —NHCH₂CH₂OCH₃;
each R_{b} is independently F, Cl, —CH₃, —CH₂OH, —CF₃, cyclopropyl, and/or —OCH₃;
y is zero, 1 or 2; and
z is zero, 1, or 2.

In one embodiment, the present invention provides a method of inhibiting tumor growth in a subject having triple-negative breast cancer (TNBC), wherein one or more cells of the TNBC tumor comprises one or more Notch-activating genetic alterations, comprising the step of administering to said subject a composition comprising one or more compounds represented by the structure of Formula (III):

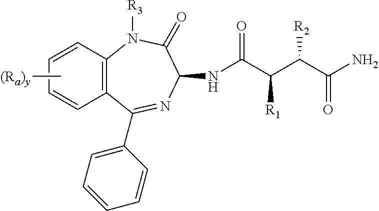

or prodrugs or salts thereof; wherein:
R₁ is —CH₂CF₃ or —CH₂CH₂CF₃;
R₂ is —CH₂CF₃, —CH₂CH₂CF₃, or —CH₂CH₂CH₂CF₃;
R₃ is H or —CH₃;
each Rₐ is independently F, Cl, —CN, —OCH₃, and/or —NHCH₂CH₂OCH₃; and
y is zero, 1, or 2.

In one embodiment, the present invention provides a method of inhibiting tumor growth in a subject having triple-negative breast cancer (TNBC), wherein one or more cells of the TNBC tumor comprises one or more Notch-activating genetic alterations, comprising the step of administering to said subject a composition comprising:

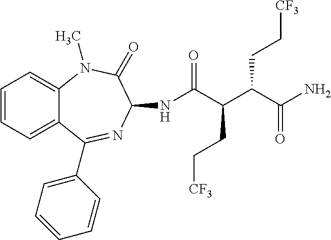 [SMILES: CN1C(=O)[C@@H](NC(=O)[C@H](CCC(F)(F)F)[C@H](CCC(F)(F)F)C(N)=O)N=C(c2ccccc2)c2ccccc21]

In one embodiment, the present invention provides a method of inhibiting tumor growth in a subject having triple-negative breast cancer (TNBC), wherein one or more cells of the TNBC tumor comprises one or more Notch-activating genetic alterations, comprising the step of administering to said subject a composition comprising:

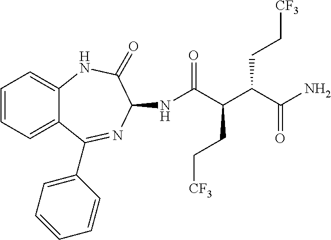 [SMILES: NC(=O)[C@@H](CCC(F)(F)F)[C@@H](CCC(F)(F)F)C(=O)N[C@H]1N=C(c2ccccc2)c2ccccc2NC1=O]

In one embodiment, inhibiting tumor growth comprises decreasing the growth of the tumor in comparison to control by 100%.

In one embodiment, the present invention provides a method of prolonging or increasing progression-free survival or overall survival in a subject having a tumor, wherein one or more cells of said tumor comprises one or more Notch-activating genetic alterations, comprising the step of administering to said subject a composition comprising one or more compounds represented by the structure of Formula (I): and/or at least one salt thereof, as described herein.

In one embodiment, the present invention provides a method of prolonging or increasing progression-free survival or overall survival in a subject having breast cancer, wherein one or more cells of said tumor comprises one or more Notch-activating genetic alterations, comprising the step of administering to said subject a composition comprising one or more compounds represented by the structure of Formula (I): and/or at least one salt thereof, as described herein.

In one embodiment, the present invention provides a method of prolonging or increasing progression-free survival or overall survival in a subject having triple-negative breast cancer (TNBC), wherein one or more cells of the TNBC tumor comprises one or more Notch-activating genetic alterations, comprising the step of administering to said subject a composition comprising one or more compounds represented by the structure of Formula (I): and/or at least one salt thereof,

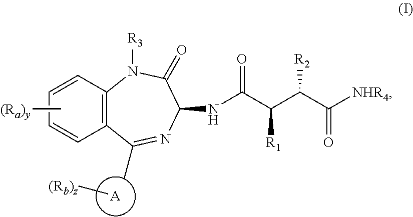

wherein:
R₁ is —CH₂CF₃ or —CH₂CH₂CF₃;
R₂ is —CH₂CF₃, —CH₂CH₂CF₃, or —CH₂CH₂CH₂CF₃;
R₃ is H, —CH₃ or Rₓ;
R₄ is H or R_{y};
Rₓ
is: —CH₂OC(O)CH(CH₃)NH₂, —CH₂OC(O)CH(NH₂)CH(CH₃)₂, —CH₂OC(O)CH((CH(CH₃)₂)NHC(O)CH(NH₂)CH(CH₃)₂,

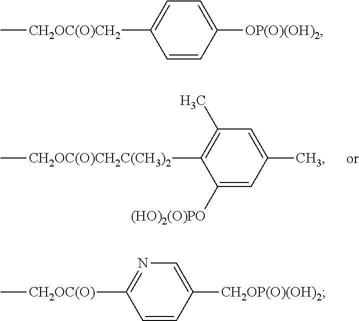

R_{y} is: —SCH₂CH(NH₂)C(O)OH, —SCH₂CH(NH₂)C(O)OH₃,
or —SCH₂CH(NH₂)C(O)OC(CH₃)₃;
Ring A is phenyl or pyridinyl;
each Rₐ is independently F, Cl, —CN, —OCH₃, C₁₋₃ alkyl, —CH₂OH, —CF₃, cyclopropyl, —OCH₃, —O(cyclopropyl) and/or —NHCH₂CH₂OCH₃;
each R_{b} is independently F, Cl, —CH₃, —CH₂OH, —CF₃, cyclopropyl, and/or —OCH₃;
y is zero, 1 or 2; and
z is zero, 1, or 2.

In one embodiment, the present invention provides a method of prolonging or extending progression-free survival or overall survival in a subject having triple-negative breast cancer (TNBC), wherein one or more cells of the TNBC tumor comprises one or more Notch-activating genetic alterations, comprising the step of administering to said subject a composition comprising one or more compounds represented by the structure of Formula (III):

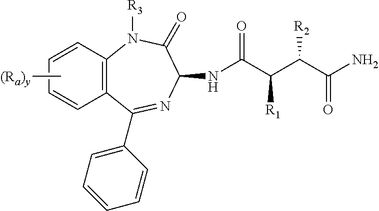

or prodrugs or salts thereof; wherein:
R₁ is —CH₂CF₃ or —CH₂CH₂CF₃;
R₂ is —CH₂CF₃, —CH₂CH₂CF₃, or —CH₂CH₂CH₂CF₃;
R₃ is H or —CH₃;
each Rₐ is independently F, Cl, —CN, —OCH₃, and/or —NHCH₂CH₂OCH₃; and
y is zero, 1, or 2.

In one embodiment, the present invention provides a method of prolonging or extending progression-free survival or overall survival in a subject having triple-negative breast cancer (TNBC), wherein one or more cells of the TNBC tumor comprises one or more Notch-activating genetic alterations, comprising the step of administering to said subject a composition comprising:

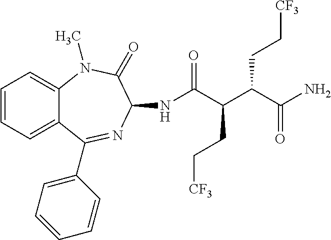 [SMILES: CN1C(=O)[C@@H](NC(=O)[C@H](CCC(F)(F)F)[C@H](CCC(F)(F)F)C(N)=O)N=C(c2ccccc2)c2ccccc21]

In one embodiment, the present invention provides a method of prolonging or increasing progression-free survival or overall survival in a subject having triple-negative breast cancer (TNBC), wherein one or more cells of the TNBC tumor comprises one or more Notch-activating genetic alterations, comprising the step of administering to said subject a composition comprising:

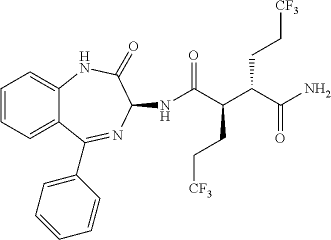 [SMILES: NC(=O)[C@@H](CCC(F)(F)F)[C@@H](CCC(F)(F)F)C(=O)N[C@H]1N=C(c2ccccc2)c2ccccc2NC1=O]

In one embodiment, any of the compositions as described herein are used in a method of increasing progression free survival (PFS). In another embodiment, any of the compositions as described herein are used in a method of increasing the duration of response (DOR). In another embodiment, any of the compositions as described herein are used in a method of increasing overall survival (OS). In another embodiment, any of the compositions as described herein are used in a method of increasing or enhancing the quality of life (QoL), which, in one embodiment, is determined by Quality of Life Instrument—Breast Cancer Patient Version (QOL-BC).

In one embodiment, the present invention provides a method of reducing tumor size or suppressing or inhibiting tumor growth in a subject having cancer, wherein said tumor or cancer does not have an activated Notch pathway, comprising the steps of administering to said subject a first composition comprising one or more compounds represented by the structure of Formula (I): and/or at least one salt thereof, as described herein and a second composition comprising an additional cytotoxic agent, which, in one embodiment, comprises an anti-cancer agent. In one embodiment, the tumor lacks a Notch GOF mutation. In one embodiment, the cancer comprises breast cancer. In one embodiment, the breast cancer comprises triple-negative breast cancer (TNBC).

In another embodiment, the present invention provides a method of reducing tumor size or suppressing or inhibiting tumor growth in a subject having breast cancer, wherein the tumor does not have an activated Notch pathway, comprising the steps of administering to said subject a first composition comprising one or more compounds represented by the structure of Formula (III) as described herein and a second composition comprising an additional cytotoxic agent. In one embodiment, the tumor lacks a Notch GOF mutation. In one embodiment, the breast cancer comprises TNBC.

In one embodiment, the anti-cancer agent comprises eribulin. In another embodiment, the anti-cancer agent comprises vinorelbine. In one embodiment, the combined therapy is administered to a subject wherein the tumor or cancer cells of said subject comprise Notch-activating genetic alterations. In another embodiment, the combined therapy is administered to a subject wherein the tumor or cancer cells of said subject do not have Notch-activating genetic alterations.

In one embodiment, the combined therapy is administered to a subject wherein said Notch-activating genetic alteration comprises a Notch GOF mutation. In another embodiment, the combined therapy is administered to a subject wherein said Notch-activating genetic alteration does not comprise a Notch GOF mutation.

In one embodiment, the triple-negative breast cancer comprises luminal triple-negative breast cancer. In another embodiment, the triple-negative breast cancer comprises basal-like triple-negative breast cancer. In one embodiment, the basal-like triple-negative breast cancer comprises immune enriched basal-like triple-negative breast cancer. In another embodiment, the basal-like triple-negative breast cancer comprises a basal triple-negative breast cancer without immune enrichment. In another embodiment, the triple-negative breast cancer comprises BRCA-mutated triple-negative breast cancer.

In one embodiment, the triple-negative breast cancer comprises luminal androgen receptor (LAR) triple-negative breast cancer. In another embodiment, the triple-negative breast cancer comprises basal-like triple-negative breast cancer. In one embodiment, the basal-like triple-negative breast cancer comprises BL1 triple-negative breast cancer. In another embodiment, the basal-like triple-negative breast cancer comprises BL2 triple-negative breast cancer. In another embodiment, the triple-negative breast cancer comprises immunomodulatory (IM) triple-negative breast cancer. In another embodiment, the triple-negative breast cancer comprises mesenchymal (M) triple-negative breast cancer. In another embodiment, the triple-negative breast cancer comprises mesenchymal stem like (MSL) triple-negative breast cancer. In another embodiment, the triple-negative breast cancer comprises unstable (UNS) triple-negative breast cancer.

In another embodiment, the cancer comprises astrocytoma, bladder cancer, breast cancer, cholangiocarcinoma (CCA), colon cancer, colorectal cancer, colorectal carcinoma, epithelial carcinoma, epithelial ovarian cancers, fibrosarcoma, gall bladder cancer, gastric cancer, glioblastoma, glioma, head and neck cancer, hepatocellular carcinoma, kidney cancer, liver cancer, lung cancer including non-small cell lung cancer (NSCLC), malignant fibrous histiocytoma (MFH), malignant pleural mesothelioma (MPM), medulloblastoma, melanoma, mesothelioma, neuroblastoma, osteosarcoma, ovarian adenocarcinoma, ovarian cancer, pancreatic adenocarcinoma, pancreatic cancer, prostate cancer, renal cell carcinoma (RCC), rhabdomyosarcoma, seminal vesicle cancer, endometrial cancer, and thyroid cancer.

As used herein, the term “cancer” includes the above categories of carcinoma, sarcoma, myeloma, leukemia, lymphoma and mixed type tumors. In particular, the term cancer includes: lymphoproliferative disorders, breast cancer, ovarian cancer, prostate cancer, cervical cancer, endometrial cancer, lung cancer, bone cancer, liver cancer, stomach cancer, bladder cancer, colon cancer, colorectal cancer, pancreatic cancer, cancer of the thyroid, head and neck cancer, cancer of the central nervous system, brain cancer, cancer of the peripheral nervous system, skin cancer, kidney cancer, as well as metastases of all the above. More particularly, as used herein the term may refer to: hepatocellular carcinoma, hematoma, hepatoblastoma, rhabdomyosarcoma, esophageal carcinoma, thyroid carcinoma, ganglioblastoma, glioblastoma, fibrosarcoma, myxosarcoma, liposarcoma, chondrosarcoma, osteogenic sarcoma, chordoma, angiosarcoma, endotheliosarcoma, Ewing's tumor, leimyosarcoma, rhabdotheliosarcoma, invasive ductal carcinoma, papillary adenocarcinoma, melanoma, basal cell carcinoma, adenocarcinoma (well differentiated, moderately differentiated, poorly differentiated or undifferentiated), renal cell carcinoma, hypernephroma, hypernephroid adenocarcinoma, bile duct carcinoma, choriocarcinoma, seminoma, embryonal carcinoma, Wilms' tumor, testicular tumor, lung carcinoma including small cell, non-small and large cell lung carcinoma, bladder carcinoma, glioma, astrocyoma, medulloblastoma, craniopharyngioma, ependymoma, pinealoma, retinoblastoma, neuroblastoma, colon carcinoma, rectal carcinoma, hematopoietic malignancies including all types of leukemia and lymphoma including: acute myelogenous leukemia, acute myelocytic leukemia, acute lymphocytic leukemia, chronic myelogenous leukemia, chronic lymphocytic leukemia, mast cell leukemia, multiple myeloma, myeloid lymphoma, Hodgkin's lymphoma, non-Hodgkin's lymphoma, Waldenstrom's Macroglobulinemia, or a combination thereof. In another embodiment, cancer comprises squamous cell carcinoma.

In another embodiment, the administration of the any of the compositions as described herein reduces the growth of the cells of a solid tumor or hematological malignancy by 40%, 50%, 60%, 70%, 80%, 90% or 95% compared to growth of the cells of the solid tumor or hematological malignancy that have not been treated with the compositions, or that have been treated with placebo, vehicle or with another anti-cancer therapy. In the case of combination treatments, the administration of any of the described combinations reduces the growth of the cells of a solid tumor or hematological malignancy compared to subjects treated with either one of the compositions, via a different cancer treatment, who have been treated with placebo or vehicle, or who have not been treated.

In another embodiment, the present invention provides methods of increasing or lengthening survival of a subject having a neoplasia. As used herein, the term “neoplasia” refers to a disease characterized by the pathological proliferation of a cell or tissue and its subsequent migration to or invasion of other tissues or organs. Neoplasia growth is typically uncontrolled and progressive, and occurs under conditions that would not elicit, or would cause cessation of, multiplication of normal cells. Neoplasias can affect a variety of cell types, tissues, or organs, including but not limited to an organ selected from the group consisting of bladder, colon, bone, brain, breast, cartilage, glia, esophagus, fallopian tube, gallbladder, heart, intestines, kidney, liver, lung, lymph node, nervous tissue, ovaries, pleura, pancreas, prostate, skeletal muscle, skin, spinal cord, spleen, stomach, testes, thymus, thyroid, trachea, urogenital tract, ureter, urethra, uterus, and vagina, or a tissue or cell type thereof. Neoplasias include cancers, such as sarcomas, carcinomas, or plasmacytomas (malignant tumor of the plasma cells).

In one embodiment, a subject as described herein is being treated with or has been previously treated with radiation therapy, chemotherapy, transplantation, immunotherapy, hormone therapy, or photodynamic therapy.

In another embodiment the present invention provides a method of treating a cancer in a subject, wherein one or more cells of said cancer comprises one or more or no genetic alterations in one or more Notch genes, wherein said genetic alteration does not activate Notch-regulated genes, comprising the step of administering to said subject a composition comprising one or more compounds represented by the structure of Formula (I), (III), (1), (2), or (22) and administering to said subject a composition comprising one or more additional anti-cancer agents.

In one embodiment, one or more of the Notch genes in the cancer cells is wild-typer. In another embodiment, the cancer cells are Notch-off and negative IHC. In another embodiment, the cancer cells have a wild-type Notch phenotype. In another embodiment, the cancer cells are not Notch activated.

In one embodiment, the term wild-type as used herein describes a wild-type gene that is not mutated. In another embodiment, the term wild-type as used herein describes a variant gene or a gene with mutations, but that maintains a wild-type Notch phenotype. In another embodiment, tumors with wild-type Notch phenotype refers to wild-type Notch function. In one embodiment, tumors with wild-type Notch phenotype may comprise Notch-related genes that have passenger mutations. In another embodiment, tumors with wild-type Notch phenotype may comprise mutations outside of the NRR and PEST hot spots.

In another embodiment, the cells of the cancer do not comprise alterations in one or more Notch genes. In another embodiment, the cancer does not overexpress Notch targets. In another embodiment, the cancer does not demonstrate a Notch signature expression. In another embodiment, the cancer does not demonstrate a 21 gene Notch signature expression, as described herein. In another embodiment, the cancer cells lack a GOF mutation. In another embodiment, the cancer cells comprise one or more Notch genes comprising one or more loss of function (LOF) mutations. In another embodiment, the cancer cell comprises one or more Notch genes that is a variant of unknown significance (VUS).

In one embodiment, the lack of activation of Notch-regulated genes is detected via a Notch activation signature, as described herein. In one embodiment, the Notch activation signature comprises a decrease or no significant change in the expression of one or more Notch-regulated genes.

### Notch-Activating Genetic Alterations

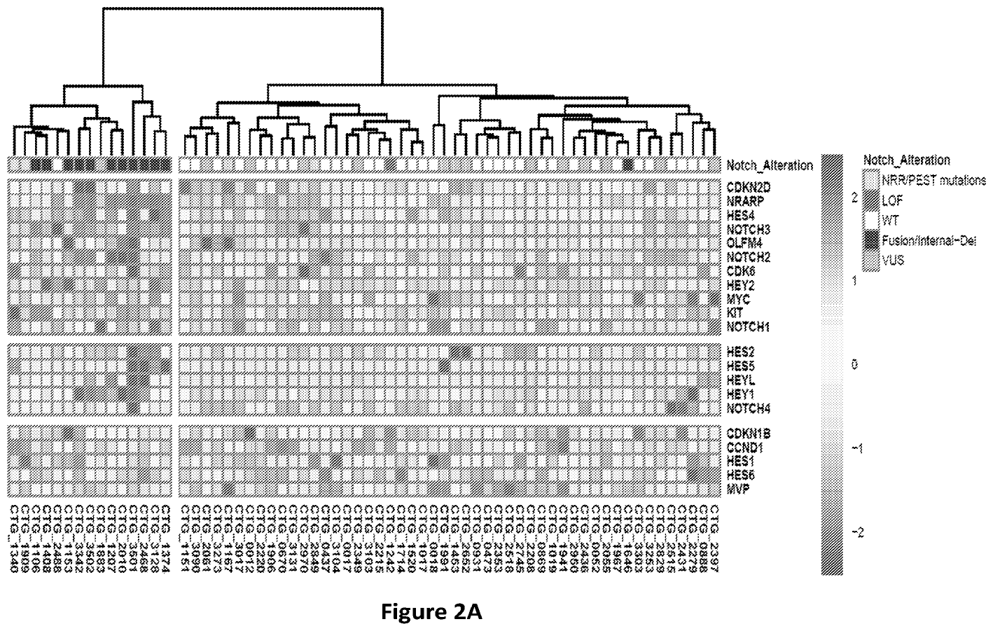

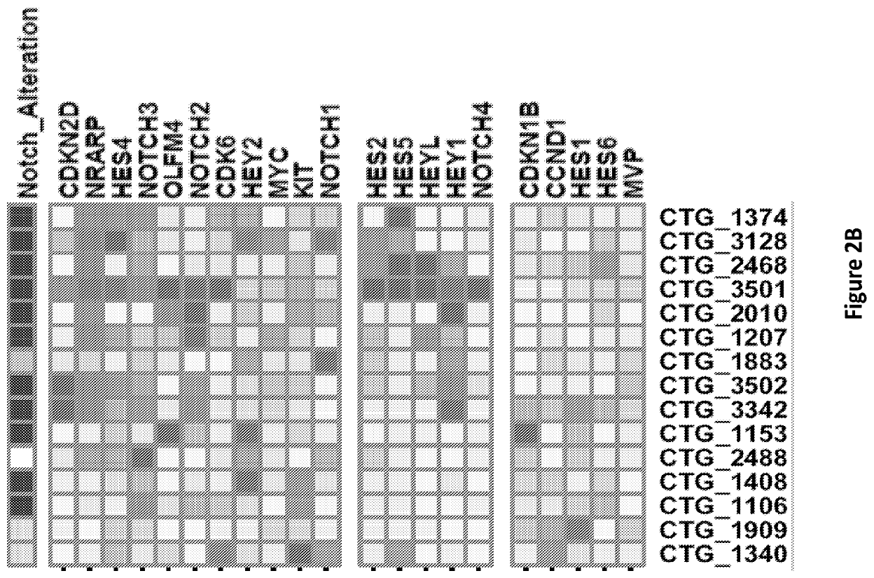

In one embodiment, a cancer as described herein comprises a Notch activating alteration. In another embodiment, a cancer as described herein comprises a Notch activating genetic alteration. In another embodiment, a cancer as described herein comprises a Notch activating mutation. In another embodiment, a cancer as described herein comprises a Notch activating genetic mutation. In another embodiment, a cancer as described herein comprises a Notch mutation. In another embodiment, a cancer as described herein comprises a Notch altering mutation. In another embodiment, a cancer as described herein comprises a Notch activation gene expression signature. In another embodiment, a cancer as described herein comprises the 21 gene Notch activation gene expression signature as described herein, for example, in FIGS. 2A-2B.

In another embodiment, a cancer as described herein is characterized by an activated Notch pathway. In another embodiment, a cancer as described herein comprises an activated Notch pathway. In another embodiment, a cancer as described herein has an activated Notch pathway.

In another embodiment, the present invention provides a method of detecting activating Notch genetic alterations in a cell or tumor cell comprising the step of evaluating the Notch activated gene expression signature, as described herein.

In one embodiment, a method as described herein comprises the step of identifying a tumor comprising an activated Notch pathway. In one embodiment, the identification step is performed prior to the administering step. In one embodiment, the identification step comprises assessing mutations or genetic alterations in one or more Notch-related genes. In another embodiment, the identification step comprises assessing mutations in one or more Notch-related genes. In another embodiment, the identification step comprises detecting activating genetic alterations. In another embodiment, the identification step comprises detecting Notch-on gene expression. In another embodiment, the identification step comprises assessing mRNA expression of Notch genes or Notch-related genes. In another embodiment, the identification step comprises assessing protein expression of Notch genes or Notch-related genes. In one embodiment, Notch-related genes are genes that are downstream of Notch and that are activated by Notch. In another embodiment, Notch-related genes are genes that are upstream of Notch and that activate Notch. In another embodiment, the identification step comprises a combination of any of the steps described hereinabove.

In another embodiment, a Notch-activating genetic alteration comprises a mutation in one or more Notch-related genes. In one embodiment, a Notch-activating genetic alteration comprises a sequence variant of one or more Notch-related genes. In one embodiment, the sequence variant comprises a fusion in in one or more Notch-related genes. In another embodiment, the genetic alteration comprises a fusion in in one or more Notch-related genes. In another embodiment, the genetic alteration comprises a gene rearrangement, which, in one embodiment, is in the ectodomain of a Notch gene. In one embodiment, the gene rearrangement removes most of the NRR, which in one embodiment, is greater than 50% of the NRR, in another embodiment, greater than 60%, 70%, 75%, 80%, 85%, 90%, 95%, 97%, 98%, 99%. In another embodiment, the gene rearrangement removes 100% of the NRR.

In one embodiment, Notch-related genes comprise Notch-regulated genes.

In one embodiment, the mutation in one or more Notch-related genes induces a gain of function (GOF) in Notch activity. In one embodiment, a subject whose cancer cells comprise one or more mutations leading to Notch GOF are administered monotherapy with a compound of Formula (I) as described herein. In another embodiment, a subject whose cancer cells comprise one or more mutations leading to Notch GOF are administered a combination therapy comprising a compound of Formula (I) as described herein and another anti-cancer compound.

In one embodiment, Notch GOF mutations are associated with one or more truncated forms of any one of the four Notch genes. In one embodiment, such truncations comprise rearrangements which, in one embodiment, remove the sequences encoding the ectodomain of the receptor. In one embodiment, these rearrangements produce Notch genes that drive the transcription of aberrant 5′-deleted transcripts encoding constitutively active polypeptides that lack the EGF-like ligand binding domain and/or parts of the NRR region.

In one embodiment, a mutation in one or more Notch-related genes comprises a mutation in a Notch gene hotspot. In one embodiment, a Notch gene hotspot comprises a negative regulatory region (NRR) domain, a proline, glutamic acid, serine and threonine rich domain (PEST) domain, or a combination thereof. In one embodiment, a mutation in one or more Notch-related genes comprises a mutation in an NRR. In one embodiment, the mutation in one or more Notch-related genes functionally inactivates the NRR of a Notch gene.

In another embodiment, a mutation in one or more Notch-related genes comprises a mutation in the PEST domain. In one embodiment, the mutation in one or more Notch-related genes functionally inactivates the PEST domain of a Notch gene. In another embodiment, a mutation in one or more Notch-related genes comprises a mutation in an NRR and a PEST domain. In one embodiment, these mutations are GOF activating mutations.

In another embodiment, the mutation in one or more Notch-related genes comprises a gene rearrangement that removes most of the Notch ectodomain, including the NRR. In one embodiment, these mutations are GOF mutations. In another embodiment, the mutation in one or more Notch-related genes comprises an internal deletion within one or more Notch genes.

In one embodiment, the mutation in one or more Notch-related genes is detected using DNA sequencing or RNA sequencing.

In another embodiment, the Notch-activating genetic alteration comprises a missense mutation. In another embodiment, the Notch-activating genetic alteration comprises a nonsense mutation. In another embodiment, the Notch-activating genetic alteration comprises an insertion. In another embodiment, the Notch-activating genetic alteration comprises a deletion. In another embodiment, the Notch-activating genetic alteration comprises a duplication. In another embodiment, the Notch-activating genetic alteration comprises a frameshift mutation. In another embodiment, the Notch-activating genetic alteration comprises a repeat expansion. In another embodiment, the Notch-activating genetic alteration comprises a gene fusion.

In another embodiment, the Notch-activating genetic alteration is manifested via a Notch activation signature. In another embodiment, the Notch-activating genetic alteration is identified by its Notch activation signature.

In one embodiment, the triple negative breast cancer comprises one or more cells with an activating Notch signature or a Notch activation signature. In one embodiment, the activating Notch signature comprises gene expression of a combination of Notch-regulated genes, which, taken together, have an overall activating effect on the Notch signaling pathway.

In one embodiment, the activated Notch pathway is identified by assessing the gene expression profile of Notch-regulated genes. In one embodiment, the gene expression profile of Notch-regulated genes comprising a Notch-activated gene signature. In another embodiment, the gene expression profile of Notch-regulated genes comprising a Notch-on activated gene signature. In another embodiment, the activated Notch pathway is identified by assessing the mRNA levels of Notch-regulated genes. In another embodiment, the activated Notch pathway is identified by assessing protein levels, which in one embodiment, is assessed using Immunohistochemical methods and, in another embodiment, a Western Blot, as is well known in the art. In another embodiment, the activated Notch pathway is identified by assessing gene mutations. In one embodiment, the gene mutations are detected using DNA sequencing. In another embodiment, the gene mutations are detected using RNA sequencing.

In one embodiment, the activating Notch signature comprises upregulation of or increase in the expression of one or more Notch-regulated genes (FIGS. 2A-B). In another embodiment, the Notch activation signature comprises an overall increase in the expression of Notch-regulated genes. In one embodiment, the Notch-regulated gene comprises HEY1, NOTCH1, HEYL, NOTCH2, OLFM4, MYC, CDK6, HEY2, KIT, NRARP, MVP, HES6, CDKN2D, NOTCH4, NOTCH3, HES4, HES5, CCND1, HES1, CDKN1B, HES2 or a combination thereof. In one embodiment, the activating Notch signature comprises a 21 gene Notch activating signature.

In one embodiment, the Notch-activating genetic alteration alters the expression of HEY1. In another embodiment, the Notch-activating genetic alteration alters the expression of NOTCH1. In another embodiment, the Notch-activating genetic alteration alters the expression of HEYL. In another embodiment, the Notch-activating genetic alteration alters the expression of NOTCH2. In another embodiment, Notch-activating genetic alteration alters the expression of OLFM4. In another embodiment, the Notch-activating genetic alteration alters the expression of MYC. In another embodiment, the Notch-activating genetic alteration alters the expression of CDK6. In another embodiment, the Notch-activating genetic alteration alters the expression of HEY2. In another embodiment, the Notch-activating genetic alteration alters the expression of KIT. In another embodiment, the Notch-activating genetic alteration alters the expression of NRARP. In another embodiment, the Notch-activating genetic alteration alters the expression of MVP. In another embodiment, the Notch-activating genetic alteration alters the expression of HES6. In another embodiment, the Notch-activating genetic alteration alters the expression of CDKN2D. In another embodiment, the Notch-activating genetic alteration alters the expression of NOTCH4. In another embodiment, the Notch-activating genetic alteration alters the expression of NOTCH3. In another embodiment, the Notch-activating genetic alteration alters the expression of HES4. In another embodiment, the Notch-activating genetic alteration alters the expression of HES5. In another embodiment, the Notch-activating genetic alteration alters the expression of CCND1. In another embodiment, the Notch-activating genetic alteration alters the expression of HES1. In another embodiment, the Notch-activating genetic alteration alters the expression of CDKN1B. In another embodiment, the Notch-activating genetic alteration alters the expression of HES2. In another embodiment, the Notch-activating genetic alteration is in any combination of the genes listed hereinabove. In one embodiment, the alteration comprises over-expression of one or more Notch-regulated genes.

In one embodiment, the Notch-related gene comprises a Notch1-related gene. In another embodiment, the Notch-related gene comprises a Notch2-related gene. In another embodiment, the Notch-related gene comprises a Notch3-related gene. In another embodiment, the Notch-related gene comprises a Notch4-related gene. In another embodiment, the Notch-related gene comprises any combination of Notch1-, Notch2-, Notch3-, and Notch4-related genes.

In another embodiment, the Notch-related gene comprises Notch1. In another embodiment, the Notch-related gene comprises Notch2. In another embodiment, the Notch-related gene comprises Notch3. In another embodiment, the Notch-related gene comprises Notch4. In another embodiment, the Notch-related gene comprises any combination of Notch1, Notch2, Notch3, and Notch4.

In one embodiment, a Notch-activating genetic alteration comprises a mutation in a gene that activates the Notch signaling pathway. In another embodiment, the Notch-related gene comprises a regulator of expression of a Notch gene.

In another embodiment, the present invention provides a Notch activation signature comprising changes in expression in the 21 Notch-related genes as described herein. In one embodiment, the Notch activation signature may be used to identify subjects responsive to treatment with Notch-regulating compounds, such as, for example, those described herein.

In another embodiment, the activated Notch pathway is identified by assessing levels of one or more Notch proteins. In one embodiment, the Notch protein levels are assessed using IHC, Western blot, or a combination thereof. In one embodiment, the level of Notch1 protein is assessed.

In one embodiment, the level of cleaved Notch1 protein is assessed. In another embodiment, the level of Notch2 protein is assessed. In another embodiment, the level of Notch3 protein is assessed. In another embodiment, the level of Notch4 protein is assessed.

In another embodiment, the mutation in one or more Notch-related genes induces a loss of function (LOF) in Notch activity. In one embodiment, a subject whose cancer cells comprise one or more mutations leading to Notch LOF are administered a combination therapy comprising a compound of Formula (I) as described herein and another anti-cancer therapy. In one embodiment, the anti-cancer therapy comprises a chemotherapy.

In another embodiment, it is not known if the mutation is a GOF or LOF Notch mutation. In one embodiment, the mutation comprises a variant of unknown significance (VUS).

### Definitions

Unless specifically stated otherwise herein, references made in the singular may also include the plural. For example, “a” and “an” may refer to either one, or one or more.

The definitions set forth herein take precedence over definitions set forth in any patent, patent application, and/or patent application publication incorporated herein by reference.

Listed below are definitions of various terms used to describe the present invention. These definitions apply to the terms as they are used throughout the specification (unless they are otherwise limited in specific instances) either individually or as part of a larger group.

As used herein, the term “administering” refers to bringing in contact with a compound of the present invention. In one embodiment, the compositions are applied locally. In another embodiment, the compositions are applied systemically. Administration can be accomplished to cells or tissue cultures, or to living organisms, for example humans.

As used herein, the terms “administering,” “administer,” or “administration” refer to deliver one or more compounds or compositions to a subject parenterally, enterally, or topically. Illustrative examples of parenteral administration include, but are not limited to, intravenous, intramuscular, intraarterial, intrathecal, intracapsular, intraorbital, intracardiac, intradermal, intraperitoneal, transtracheal, subcutaneous, subcuticular, intraarticulare, subcapsular, subarachnoid, intraspinal and intrasternal injection and infusion. Illustrative examples of enteral administration include, but are not limited to oral, inhalation, intranasal, sublingual, and rectal administration. Illustrative examples of topical administration include, but are not limited to, transdermal and vaginal administration. In particular embodiments, an agent or composition is administered parenterally, optionally by intravenous administration or oral administration to a subject.

In one embodiment, a composition of the present invention comprises a pharmaceutically acceptable composition. In one embodiment, the phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

In one embodiment, a composition of the present invention is administered in a therapeutically effective amount. In one embodiment, a “therapeutically effective amount” is intended to include an amount of a compound of the present invention alone or an amount of the combination of compounds claimed or an amount of a compound of the present invention in combination with other active ingredients effective to act as an inhibitor to a NOTCH receptor, effective to inhibit gamma secretase, or effective to treat or prevent proliferative diseases such as cancer. In one embodiment, a “therapeutically effective amount” of a composition of the invention is that amount of composition which is sufficient to provide a beneficial effect to the subject to which the composition is administered.

As used herein, “treating” or “treatment” cover the treatment of a disease-state in a subject, particularly in a human, and include: (a) preventing the disease-state from occurring in a subject, in particular, when such mammal is predisposed to the disease-state but has not yet been diagnosed as having it; (b) inhibiting the disease-state, i.e., arresting its development; and/or (c) relieving the disease-state, i.e., causing regression of the disease state.

In one embodiment, “treating” refers to, in one embodiment, therapeutic treatment and, in another embodiment, prophylactic or preventative measures. In one embodiment, the goal of treating is to prevent or lessen the targeted pathologic condition or disorder as described hereinabove. Thus, in one embodiment, treating may include directly affecting or curing, suppressing, inhibiting, preventing, reducing the severity of, delaying the onset of, reducing symptoms associated with the disease, disorder or condition, or a combination thereof. Thus, in one embodiment, “treating” refers inter alia to delaying progression, expediting remission, inducing remission, augmenting remission, speeding recovery, increasing efficacy of or decreasing resistance to alternative therapeutics, or a combination thereof. In one embodiment, “preventing” refers, inter alia, to delaying the onset of symptoms, preventing relapse to a disease, decreasing the number or frequency of relapse episodes, increasing latency between symptomatic episodes, or a combination thereof. In one embodiment, “suppressing” or “inhibiting”, refers inter alia to reducing the severity of symptoms, reducing the severity of an acute episode, reducing the number of symptoms, reducing the incidence of disease-related symptoms, reducing the latency of symptoms, ameliorating symptoms, reducing secondary symptoms, reducing secondary infections, prolonging patient survival, or a combination thereof.

In one embodiment, the term “decreasing the size of the tumor” as used herein is assessed using the “Response Evaluation Criteria In Solid Tumors” (RECIST). In one embodiment, RECIST measures reduction in tumor size by measuring the longest dimension of a target lesion. In one embodiment, the target lesion is selected on the basis of its size (lesion with the longest diameter) and its suitability for accurate repeated measurements (either by imaging techniques or clinically). In one embodiment, all other lesions (or sites of disease) are identified as non-target lesions and are also recorded at baseline. Measurements of these lesions are not required, but the presence or absence of each is noted throughout follow-up.

In one embodiment, the term “decreasing the volume of the tumor” as used herein is assessed using the radiological tumor response evaluation criteria. In one embodiment, the maximum diameter (width) of the tumor is measured in two dimensions in the translation plane and its largest perpendicular diameter on the same image (thickness), according to the World Health Organization (WHO).

According to any of the methods of the present invention and in one embodiment, a subject as described herein is human. In another embodiment, the subject is a mammal. In another embodiment, the subject is a primate, which in one embodiment, is a non-human primate. In another embodiment, the subject is murine, which in one embodiment is a mouse, and, in another embodiment is a rat. In another embodiment, the subject is canine, feline, bovine, equine, caprine, ovine, porcine, simian, ursine, vulpine, or lupine. In one embodiment, the subject is a chicken or fish.

In one embodiment, the compositions as described herein comprise the components of the composition (i.e., one or more compounds of Formula (I)) as described herein. In another embodiment, the compositions as described herein consist of the components of the composition (i.e., one or more compounds of Formula (I)) as described herein). In another embodiment, the compositions as described herein consist essentially of the components of the composition (i.e., one or more compounds of Formula (I)) as described herein.

It is to be understood that the compositions and methods of the present invention comprising the elements or steps as described herein may, in another embodiment, consist of those elements or steps, or in another embodiment, consist essentially of those elements or steps. In some embodiments, the term “comprise” refers to the inclusion of the indicated active agents, such as the gamma secretase inhibitor, as well as inclusion of other active agents, and pharmaceutically or physiologically acceptable carriers, excipients, emollients, stabilizers, etc., as are known in the pharmaceutical industry. In some embodiments, the term “consisting essentially of” refers to a composition, whose only active ingredients are the indicated active ingredients. However, other compounds may be included which are for stabilizing, preserving, etc. the formulation, but are not involved directly in the therapeutic effect of the indicated active ingredients. In some embodiments, the term “consisting essentially of” may refer to components which facilitate the release of the active ingredient. In some embodiments, the term “consisting” refers to a composition, which contains the active ingredients and a pharmaceutically acceptable carrier or excipient.

In one embodiment, “genetic alterations” as described herein comprise changes in nucleic acid sequence. In another embodiment, genetic alterations comprise changes in DNA sequence. In another embodiment, genetic alterations comprise changes in RNA sequence.

### Timing and Site of Administration

In one embodiment, in the methods of the present invention, the administration of one or more anti-cancer agents occurs prior to the administration of the compound of Formula (I). In another embodiment, in the methods of the present invention, the administration of one or more anti-cancer agents occurs concurrent with the administration of the compound of Formula (I). In another embodiment, in the methods of the present invention, the administration of one or more anti-cancer agents occurs following the administration of the compound of Formula (I). In one embodiment, concurrent administration comprises administering a single composition comprising the anti-cancer agent and compound of Formula (I). In another embodiment, concurrent administration comprises administering separate compositions.

In one embodiment, the administration of the anti-cancer agents occurs at the same site as the administration of the compound of Formula (I).

In one embodiment, the compound of Formula (I) is administered several days before and after the administration of the anti-cancer agent. In one embodiment, the compound of Formula (I) is administered 1, 2, 3, 4, or 5 days prior to the administration of the anti-cancer agent. In one embodiment, the compound of Formula (I) is administered 1, 2, 3, 4, or 5 days subsequent to the administration of the anti-cancer agent. In another embodiment, the compound of Formula (I) is administered one day before and up to 9 days following anti-cancer agent administration. In another embodiment, the compound of Formula (I) is administered one day before and on days 1, 8, and 9 following anti-cancer agent administration. In another embodiment, the compound of Formula (I) is administered one day before and 9 days following anti-cancer agent administration. In another embodiment, the compound of Formula (I) is administered one day before and daily for 9 days following anti-cancer agent administration. In another embodiment, the compound of Formula (I) is administered one day before and on day 9 following anti-cancer agent administration.

In some embodiments, one or more compositions of the present invention are administered at least once during a treatment cycle. In some embodiments, the compositions of the present invention are administered to the subject on the same days. In some embodiments, the compositions of the present invention are administered to the subject on the different days. In some embodiments, one or more compositions of the present invention are administered to the subject on the same days and on different days according to treatment schedules.

In particular embodiments, one or more compositions of the present invention are administered to the subject over one or more treatment cycles. A treatment cycle can be at least two, at least three, at least four, at least five, at least six, at least seven, at least 14, at least 21, at least 28, at least 48, or at least 96 days or more. In one embodiment, a treatment cycle is 28 days. In certain embodiments, the compositions are administered over the same treatment cycle or concurrently over different treatment cycles assigned for each composition. In various embodiments, the treatment cycle is determined by a health care professional based on conditions and needs of the subject.

In some embodiments, a composition is administered on at least one day, at least two days, at least three days, at least four days, at least five days, at least six days, at least seven days, at least eight days, at least nine days, at least ten days, at least eleven days, at least twelve days, at least 13 days, at least 14 days, at least 21 days, or all 28 days of a 28 day treatment cycle. In particular embodiments, a composition is administered to a subject once a day. In other particular embodiments, a composition is administered twice a day.

In one embodiment, one or more of the compositions as described herein are administered in one to four doses per day. In one embodiment, one or more of the compositions as described herein are administered once per day. In another embodiment, one or more of the compositions as described herein are administered twice per day. In another embodiment, one or more of the compositions as described herein are administered three times per day. In another embodiment, one or more of the compositions as described herein are administered four times per day. In another embodiment, one or more of the compositions as described herein are administered once every two days, once every three days, twice a week, once a week, once every 2 weeks, once every 3 weeks.

In one embodiment, one or more of the compositions as described herein are administered for 7 days to 28 days. In another embodiment, one or more of the compositions as described herein are administered for 7 days to 8 weeks. In another embodiment, one or more of the compositions as described herein are administered for 7 days to 50 days. In another embodiment, one or more of the compositions as described herein are administered for 7 days to six months. In another embodiment, one or more of the compositions as described herein are administered for 7 days to one and half years. In another embodiment, one or more of the compositions as described herein are administered for 14 days to 12 months. In another embodiment, one or more of the compositions as described herein are administered for 14 days to 3 years. In another embodiment, one or more of the compositions as described herein are administered for several years. In another embodiment, one or more of the compositions as described herein are administered for one month to six months.

In one embodiment, one or more of the compositions as described herein are administered for 7 days. In another embodiment, one or more of the compositions as described herein are administered for 14 days. In another embodiment, one or more of the compositions as described herein are administered for 21 days. In another embodiment, one or more of the compositions as described herein are administered for 28 days. In another embodiment, one or more of the compositions as described herein are administered for 50 days. In another embodiment, one or more of the compositions as described herein are administered for 56 days. In another embodiment, one or more of the compositions as described herein are administered for 84 days. In another embodiment, one or more of the compositions as described herein are administered for 90 days. In another embodiment, one or more of the compositions as described herein are administered for 120 days.

The number of times a composition is administered to a subject in need thereof depends on the discretion of a medical professional, the disorder, the severity of the disorder, and the subject's response to the formulation. In some embodiments, a composition disclosed herein is administered once to a subject in need thereof with a mild acute condition. In some embodiments, a composition disclosed herein is administered more than once to a subject in need thereof with a moderate or severe acute condition. In the case wherein the subject's condition does not improve, upon the doctor's discretion the composition may be administered chronically, that is, for an extended period of time, including throughout the duration of the subject's life in order to ameliorate or otherwise control or limit the symptoms of the subject's disease or condition.

In the case wherein the subject's status does improve, upon the doctor's discretion the composition may administered continuously; or, the dose of drug being administered may be temporarily reduced or temporarily suspended for a certain length of time (i.e., a “drug holiday”). The length of the drug holiday varies between 2 days and 1 year, including by way of example only, 2 days, 3 days, 4 days, 5 days, 6 days, 7 days, 10 days, 12 days, 15 days, 20 days, 28 days, 35 days, 50 days, 70 days, 100 days, 120 days, 150 days, 180 days, 200 days, 250 days, 280 days, 300 days, 320 days, 350 days, and 365 days. The dose reduction during a drug holiday may be from 10%-100%, including by way of example only 10%, 15%, 20%, 25%, 30%, 35%, 40%, 45%, 50%, 55%, 60%, 65%, 70%, 75%, 80%, 85%, 90%, 95%, and 100%.

### Kits

The present invention further comprises combinations of the compositions of the present invention and, optionally, one or more additional agents in kit form, e.g., where they are packaged together or placed in separate packages to be sold together as a kit, or where they are packaged to be formulated together.

In certain embodiments, the kit comprises a therapeutic or prophylactic composition containing an effective amount of the compound of Formula (I), as described herein, which in one embodiment, comprises 4 mg of the compound of Formula (I). In certain embodiments, the kit comprises a sterile container which contains therapeutic or prophylactic agents; such containers can be boxes, ampules, bottles, vials, tubes, bags, pouches, blister-packs, or other suitable container forms known in the art. Such containers can be made of plastic, glass, laminated paper, metal foil, or other materials suitable for holding medicaments.

If desired, the composition(s) are provided together with instructions for administering the composition(s) to a subject having triple-negative breast cancer (TNBC). The instructions will generally include information about the use of the composition for reducing tumor size or volume or suppressing or inhibiting tumor growth. In other embodiments, the instructions include at least one of the following: description of the therapeutic agent; dosage schedule and administration for reducing tumor size or volume or suppressing or inhibiting tumor growth; precautions; warnings; indications; counter-indications; overdosage information; adverse reactions; animal pharmacology; clinical studies; and/or references. The instructions may be printed directly on the container (when present), or as a label applied to the container, or as a separate sheet, pamphlet, card, or folder supplied in or with the container.

In another embodiment, the present invention provides a method of identifying a candidate subject for treatment with a compound represented by the structure of Formula (III), (IV), (1), or (2), as described herein comprising the step of evaluating Notch gene function in said subject. In one embodiment, evaluating Notch gene function comprises determining if there are Notch mutations. In one embodiment, the Notch mutations are in a PEST region of a Notch gene. In another embodiment, the Notch mutations are in the NRR of a Notch gene. In another embodiment, evaluating Notch gene function comprises determining the expression of Notch-regulated genes. In one embodiment, the genes are downstream of Notch in the Notch signaling pathway.

In another embodiment, the present invention further provides a kit for identifying a candidate subject for treatment with a compound represented by the structure of Formula (III), (IV), (1), or (2), as described herein comprising an evaluator of Notch gene function. In one embodiment, the evaluator comprises RNA-seq or another RNA sequencing tool to reveal the presence and quantity of RNA in a biological sample at a given moment. In another embodiment, other methods of evaluating the quantity of downstream Notch protein RNA may be utilized, as are well known in the art. In another embodiment, the evaluator comprises a DNA sequencing method, as are known in the art. In one embodiment, instructions for use are included in the kit.

In another embodiment, the present invention provides a method of treating a proliferative disorder in a subject comprising the steps of a) evaluating Notch gene function; and b) treating said subject with a compound represented by the structure of Formula (III), (IV), (1), or (2), as described herein, if the result of step a) indicates that proliferative cells comprise a Notch gain of function phenotype. In one embodiment, Notch gene function is evaluated by detecting RNA expression of Notch genes that are expressed in healthy tissue corresponding to the proliferative tissue. In another embodiment, Notch gene function is evaluated by sequencing DNA of Notch genes, wherein if said DNA comprises a mutation in or near PEST region or NRR of a Notch gene, then said Notch gene function is considered a GOF.

In another embodiment, the present invention further provides a kit for treating a proliferative disorder in a subject comprising a) an evaluator of Notch gene function and b) a composition comprising a compound represented by the structure of Formula (III), (IV), (1), or (2), as described herein, and optionally another composition comprising an anti-cancer therapeutic compound. In one embodiment, the evaluator comprises RNA-seq or another RNA sequencing tool to reveal the presence and quantity of RNA in a biological sample at a given moment. In another embodiment, other methods of evaluating the quantity of downstream Notch protein RNA may be utilized, as are well known in the art. In another embodiment, the evaluator comprises a DNA sequencing method, as are known in the art. In one embodiment, instructions for use are included in the kit.

While certain features of the invention have been illustrated and described herein, many modifications, substitutions, changes, and equivalents will now occur to those of ordinary skill in the art. It is, therefore, to be understood that the appended claims are intended to cover all such modifications and changes as fall within the true spirit of the invention.

### EXAMPLES

### Example 1

### Targeting Notch Altered TNBC PDX Models with Compound (1)

### Methods

In vivo studies: TNBC PDX tumors (Champions Oncology Inc.) of known Notch gene characterization via Next Generation Sequencing (RNA-Seq, DNA WES), were used in this study. Tumors were implanted in female athymic nude mice. Once tumors reached a size of 150-300 mm³, mice (n=5/group) were randomized to Vehicle or Compound (1) treatment arms (3 mg/kg, PO, 4on/3off) until tumors reached 1500 mm³ or day 60.

Immunohistochemistry (IHC): Formalin-Fixed Paraffin Embedded (FFPE) slides of tumors were stained using commercially available Notch antibodies (described below). FFPE tissues were cut onto charged slides at 4 μm thickness and baked at 60° C. for 1 hr. IHC was done on a Leica Bond III immunostainer following Epitope Retrieval 2 for 40 min. Staining for total NOTCH2 levels were assessed by IHC using D76A6 XP® Rabbit mAb (#5732, Cell Signaling Technology). Staining for cleaved Notch1 (Val1744) levels were assessed by IHC using D3B8 Rabbit mAb (#4147, Cell Signaling Technology). Staining with diaminobenzidine (DAB) was performed using the Polymer Refine Detection Kit (Leica). Slides were counterstained with hematoxylin. The stain results were reviewed and called by a board-certified pathologist.

Gene expression analysis: Available tumor RNA-seq data was aligned to the reference human genome (hg19) using STAR (PMID: 23104886). RNA-seq data was analyzed for the gene expression levels of 21 genes that have been published as downstream targets of Notch mediated transcription. Moreover, the 4 Notch genes are part of this list since it has been reported that some Notch genes can directly transcribe other Notch genes. Gene expression levels were calculated using featureCounts (PMID: 24227677). Samples were normalized to each other using DESeq2 (PMID: 25516281). Hierarchical clustering was preformed using Euclidean distance with Ward's method for linkage.

Notch Fusion detection: RNA-seq data from tumors was used to detect expressed Notch fusion genes using the fusion-catcher bioinformatic pipeline.

Notch Internal deletion detection: RNA-seq data cannot be used effectively for detection of large Notch intra-genic deletions. Tumors seen to have a Notch-on gene expression signal (left cluster, FIG. 2) but lacking detectable fusions or NRR/PEST mutations were subject to targeted high depth RNA sequencing (Archer, Inc.). This type of sequencing detected Notch1 internal deletions (Exon1-exon28 fusion) in CTG-3128 and CTG-2468.

### Results

### Compound (1) is Most Effective on TNBC Tumors with Notch Activation

A list of 21 Notch target genes was evaluated to create a gene expression signature-profile for Notch activation (“Notch On”) in a cohort of 64 TNBC PDX tumor models. 14 of the models (˜22%) bearing the Notch-on signature (FIG. 2A left side, FIG. 2B), were enriched with Notch1, Notch2, Notch3, and/or Notch4 genetic alterations such as rearrangements (fusions, activating internal deletions) as well as missense mutations in the NRR and PEST domains (known hotspots for activating mutations). Most of the Notch-on tumors had a strong corresponding Notch Immunohistochemistry stain (IHC-FFPE) whereas the five Notch-off tumors lacked any IHC signal (Table 1). These results strongly suggest that the Notch-on signature is indeed detecting tumors with an active Notch pathway.

Ten TNBC PDX models with known Notch genetic status were selected for study, five with Notch variants of unknown significance (VUS) missense mutations (not expected to be GOF, because mutations were not in NRR/PEST) and five with NRR/PEST mutated/rearranged (M/R) Notch genes. Of the 10 models, five had a predicted “Notch-on” 21 gene expression signature and were expected to respond to Compound (1), and five lacked this signature and were predicted to be non-responsive to Compound (1).

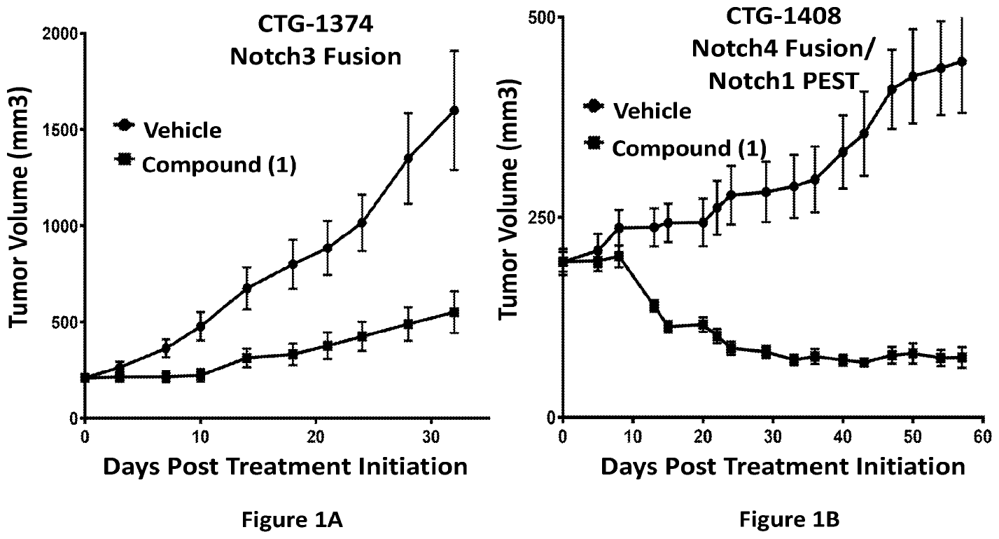

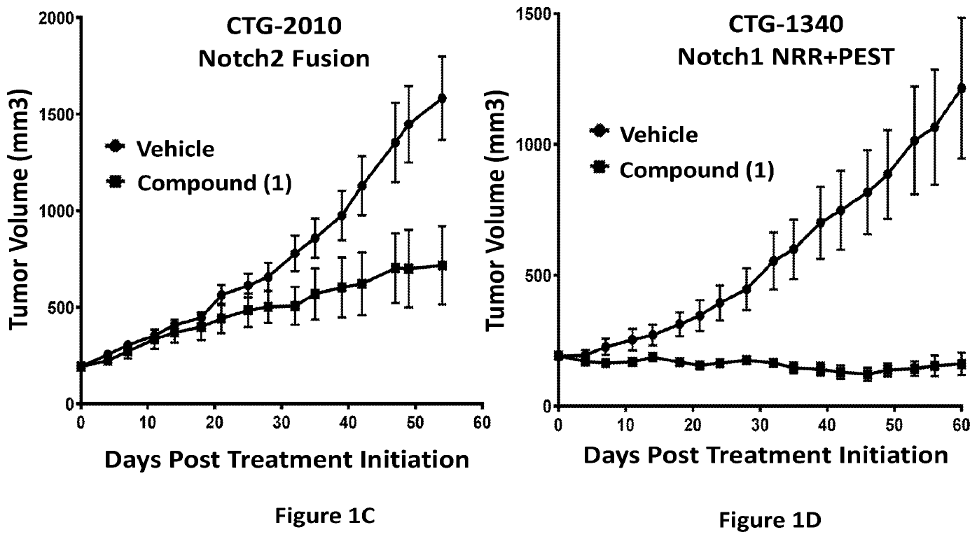

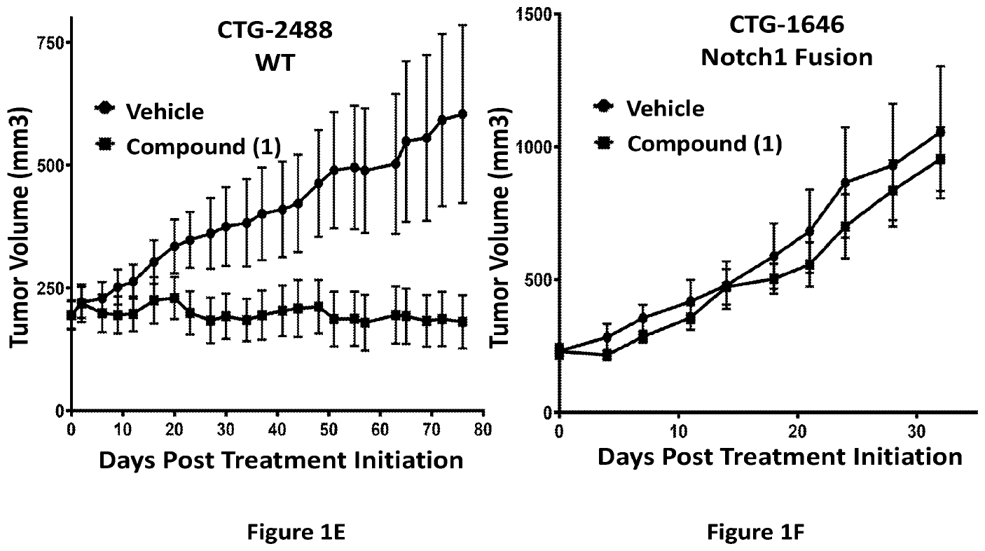

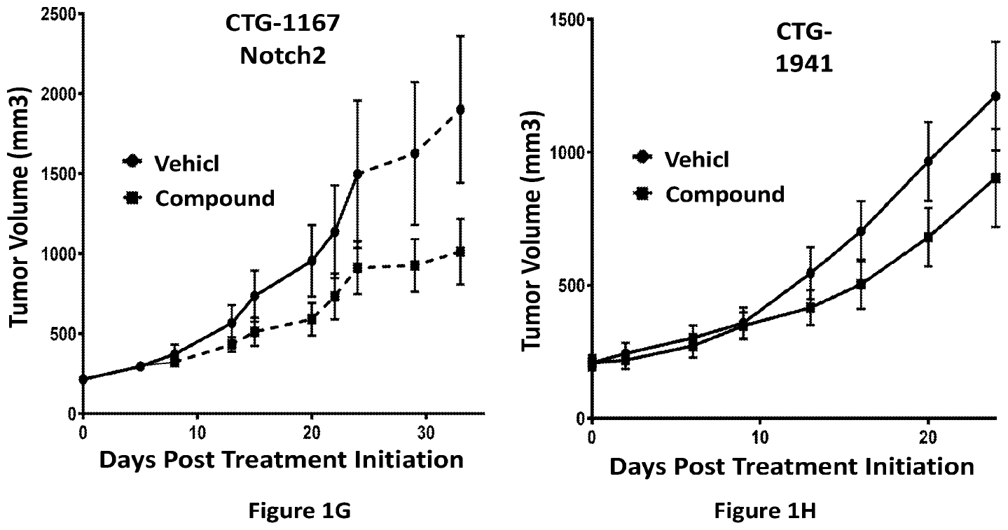

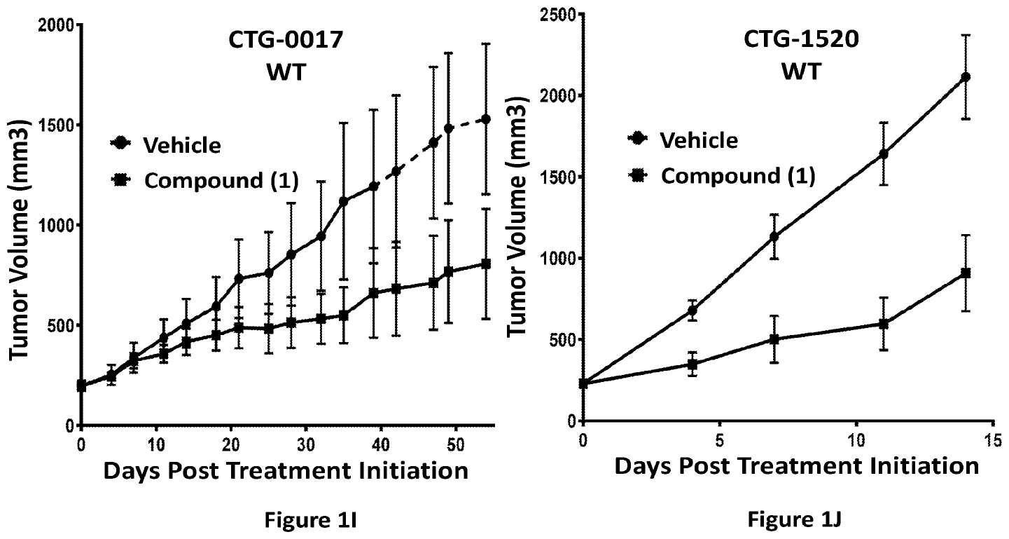

Compound (1) was more potent than vehicle in inhibiting the growth of tumors with a putative Notch-on signature, resulting in high % TGI responses (Tumor Growth Inhibition) (FIGS. 1A-E, Table 1). Within the five Notch-on signature models examined, mutation/rearrangement (M/R) in Notch genes were present in Notch1 (CTG-1340, 106% TGI p<0.0001); Notch2 (CTG-2010, 62% TGI p=0.036); Notch3 (CTG-1374, 75% TGI p=0.0174); or Notch4/Notch1 (CTG-1408, 147% TGI p<0.0001) (Table 1). CTG-2488, one of the PDX models bearing a Notch-on signature, responded well to Compound (1), but had no rearranged Notch genes or Notch mutations (WT Notch genes) (103% TGI p<0.0001). Sequencing (targeted Notch RNAseq and DNA WES) also did not reveal any known activating Notch alterations. This suggests that CTG-2488 may contain an as of yet unknown type of activating Notch gene alteration or that the pathway is being activated by events upstream to Notch, and results in a Notch-on signature. In contrast, treatment of tumors lacking the Notch-on signature with Compound (1) was no more effective than treatment with vehicle in most cases (FIGS. 1F-1J, Table 1: Notch WT (CTG-1520, 64% TGI p=0.13); Notch1 with a predicted loss of function fusion (CTG-1646, 12% TGI p=0.53), Notch VUS (CTG-1941, 30% TGI p=0.44), Notch2 PEST (CTG-1167, 45% TGI p=0.17) and Notch WT (CTG-0017, 43% TGI p=0.0104).

TABLE 1
Summary of TNBC PDX Characteristics and Response to Compound (1)

| | | Predicted | Notch-on | | | Compound |
|---|---|---|---|---|---|---|
| | | Mutation | Activation | IHC | % TGI, | (1) |
| Model | Mutation | Outcome | Signature * | Positive | P value | response |
| CTG-1340 | Notch1 | Potential | Yes | Notch1 | 106%, | High |
| | NRR + | GOF | | | <0.0001 | |
| | PEST | | | | | |
| CTG-1374 | Notch3 | ? | Yes | TBD | 75.5%, | High |
| | Fusion | | | | 0.0174 | |
| CTG-1408 | Notch4 | ? | Yes | Notch1 | 147%, | High |
| | Fusion | Potential | | | <0.0001 | |
| | Notch1 | GOF | | | | |
| | PEST | | | | | |
| CTG-2010 | Notch2 | ? | Yes | Notch2 | 62%, | Moderate |
| | Fusion | | | | 0.036 | |
| CTG-2488 | WT | Non- | Yes | TBD | 103% | High |
| | | GOF | | | <0.0001 | |
| CTG-3128 | Notch1 ** | Potential | Yes | Notch1 | TBD | TBD |
| | ECD | GOF | | | | |
| | deletion | | | | | |
| CTG-2468 | Notch1 ** | Potential | Yes | Notch1 | TBD | TBD |
| | ECD | GOF | | Notch2 | | |
| | deletion | | | | | |
| CTG-3501 | Notch2 | ? | Yes | Notch2 | TBD | TBD |
| | Fusion | | | | | |
| CTG-3502 | Notch2 | ? | Yes | Notch2 | TBD | TBD |
| | Fusion | | | | | |
| CTG-3342 | Notch2 | ? | Yes | Notch2 | TBD | TBD |
| | Fusion | | | | | |
| CTG-1153 | Notch2 | ? | Yes | Notch2 | TBD | TBD |
| | Fusion | | | | | |
| CTG-1106 | Notch2 | ? | Yes | Notch2 | TBD | TBD |
| | Fusion | | | | | |
| CTG-1909 | Notch1 | Potential | Yes | Notch1 | TBD | TBD |
| | PEST | GOF | | | | |
| CTG-1883 | VUS | Non- | Yes | Notch1 | TBD | TBD |
| | | GOF | | | | |
| CTG-1646 | Notch1 | LOF | No | Negative | 12%, NS | Inactive |
| | Fusion out | | | | 0.53 | |
| | of frame | | | | | |
| CTG-1167 | Notch2 | Potential | No | Negative | 45%, NS | Inactive |
| | PEST | GOF | | | 0.44 | |
| CTG-1941 | VUS | Non- | No | Negative | 30%, NS | Inactive |
| | | GOF | | | 0.44 | |
| CTG-0017 | WT | Non- | No | Negative | 43%, | Inactive |
| | | GOF | | | 0.0104 | |
| CTG-1520 | WT | Non- | No | Negative | 64%, NS | Moderate |
| | | GOF | | | 0.13 | |
| CTG-1207 | Notch2 | ? | Yes | TBD | TBD | TBD |
| (ER+/PR+/ | Fusion | | | | | |
| HER2−) | | | | | | |

NRR, PEST: Notch mutation hotspot domains (NRR stronger than PEST); LOF: Loss of Function; GOF: Gain of Function; VUS: Variant of unknown significance; % TGI: Tumor Growth Inhibition (high >75%); ECD: extracellular domain
* Putative Notch activation signature based on gene expression of 21 Notch target genes
** Internal deletion of exons 2-27 in Notch1 gene which disrupts the NRR and has been described as a strong GOF event. The detection of this alteration was done retrospectively by targeted RNA sequencing of the Notch genes. This type of alteration has been documented in TNBC cell lines HCC1599 and MDA-MB157.

The Notch activation gene expression signature correlated with a positive response to Compound (1) in Patient-Derived Xenograft (PDX) models of TNBC that were taken from Champions' TumorGraft® Database (FIGS. 2A-2B, Table 1). The activating Notch gene expression signature (pattern) cluster is enriched with Notch fusions & GOF mutations.

Tumors from PDX models which harbor a Notch-on signature (FIG. 2B), such as CTG-1374, CTG-1408, CTG-1340, CTG-2488 and CTG-2010 were more likely to be inhibited by treatment with Compound (1) (Table 1). Other TNBC tumors bearing the Notch-on signature, are also expected to be inhibited by treatment with Compound (1). Compound (1) treatment did not significantly inhibit tumor growth in TNBC tumors such as CTG-1646, CTG-1167 and CTG-1941, which do not harbor the Notch-on signature (FIG. 2A, center and right side).

The results demonstrated that in TNBC PDX models, the presence of a Notch-on activating gene expression signature and/or Notch activating genetic alterations, correlates with a positive Notch IHC stain and a potent response to Compound (1). Furthermore, the Notch-on gene expression signature can also be used to identify non-TNBC breast cancers with activating Notch genetic alterations. For example, CTG-1207 (a ER+/PR+/HER2− tumor) clusters together with the Notch-on TNBC tumors and contains a Notch2 Fusion (FIG. 2B).

### Example 2

### Combination Treatment with Compound (1) and Eribulin Prevents Tumor Re-Growth in Notch-Activated TNBC PDX Model

Notch-activated TNBC PDX tumors (CTG-1374) were implanted into mice. Treatment with either vehicle, Compound (1) (3 mg/kg PO 4on/3off)), Eribulin (0.5 mg/kg IV QW) or the combination of Compound (1) & Eribulin was initiated when the average tumor volume was ˜200 mm³.

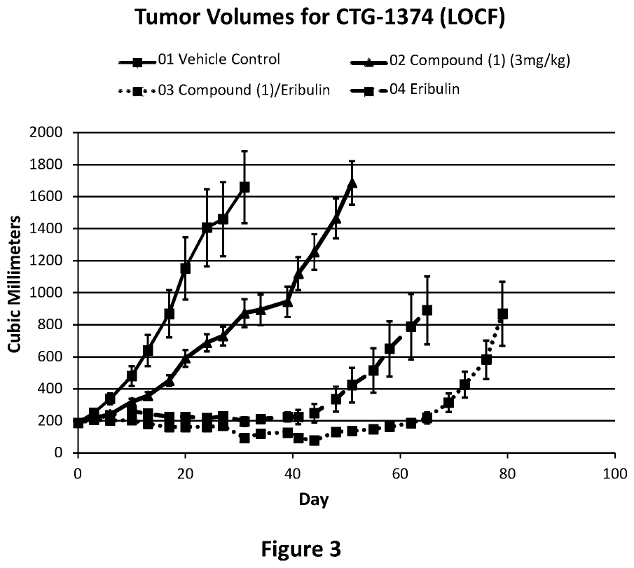

By day 28, there was a better partial response (subjects with >30% reduction in tumor volume compared to day 0) to the drug combination compared to Eribulin administration alone, with 11/13 vs 5/15 PRs, respectively (FIG. 3, Table 3).

Treatment was halted on day 28. The rate of outgrowth of tumors that had been treated with the combination of Compound (1) and Eribulin was delayed compared to the rate of outgrowth of tumors that had been treated with Eribulin alone (FIG. 3, Table 2), indicating a benefit of combined treatment with Compound (1) and Eribulin.

Once the outgrowth arms reached an average tumor volume of ˜650 mm³, the mice were re-randomized into 2 treatment arms with Eribulin alone (0.25 mg/kg IV QW), or Compound (1) (3 mg/kg PO 4on/3off in combination with Eribulin.

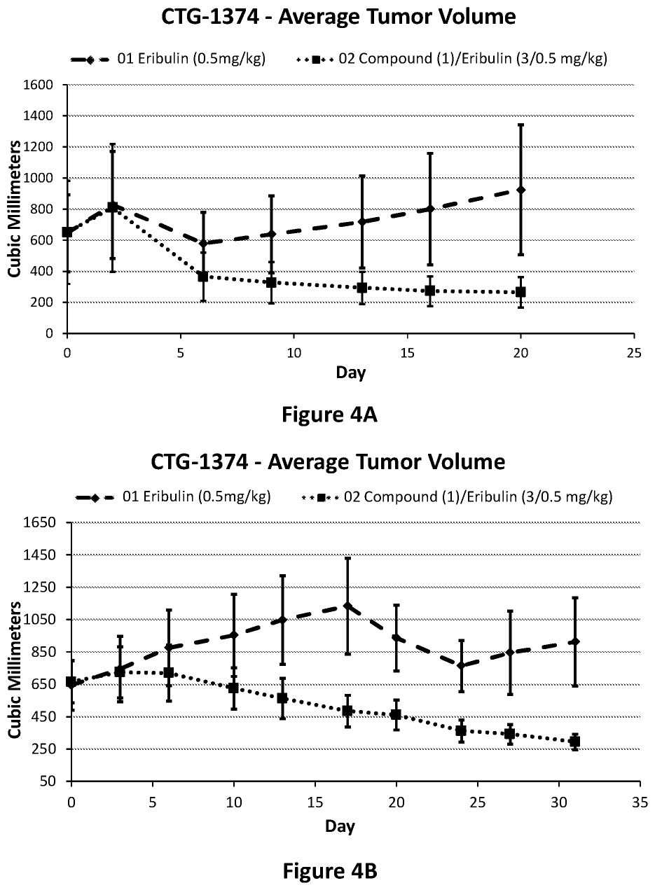

Administration of the combination of Compound (1) and Eribulin compared to Eribulin alone in TNBC PDX tumor-implanted mice previously treated with Compound (1) and Eribulin (FIG. 4B) or previously treated with Eribulin alone (FIG. 4A), not only halted tumor growth, but resulted in tumor regression (FIGS. 4A & 4B).

In contrast, administration of Eribulin alone to TNBC PDX tumor-implanted mice previously treated with Compound (1) and Eribulin (FIG. 4B) or previously treated with Eribulin alone (FIG. 4A) did not prevent further tumor growth.

TABLE 2
Average tumor volume in re-growth phase (mm³)

| | Mice/group | Day 39* | Day 51 | Day 65 |
|---|---|---|---|---|
| Compound | 15 | 944 | 1686 | NA |
| (1) | | | | |
| Eribulin | 15 | 225 | 422 | 890 |
| Compound | 13 | 125 | 136 | 219 |
| (1) + | | | | |
| Eribulin | | | | |

TABLE 3
Response in treatment and re-growth phases

| | #mice/ | | | |
|---|---|---|---|---|
| | Response | CR | PR | PD |
| Treatment Phase | Compound (1) | None | None | 15/15 |
| | Eribulin | None | 5/15 | 5/15 |
| | Compound (1) + | None | 11/13 | None |
| | Eribulin | | | |
| Re-growth Phase | Eribulin | 0/15 | 3/15 | 11/15 |
| (No treatment) | Compound (1) + | 1/13 | 1/13 | 2/13 |
| | Eribulin | | | |

These data demonstrate that the combination of Compound (1) with Eribulin slows tumor regrowth after cessation of treatment in a Notch-activated TNBC PDX model. Combined treatment of TNBC mice with Compound (1) and Eribulin additionally has beneficial effects on tumor regression after treatment re-initiation in mice previously treated with Eribulin.

### Example 3

### Compound (1) Clinical Study Protocol for TNBC

### Study Rationale and Hypothesis

Compound (1) is a potent and selective inhibitor of gamma secretase-mediated Notch signaling that is currently under development as an antitumor/antiangiogenic agent as monotherapy for the treatment of various cancers. A large body of experimental evidence supports the causal role of Notch pathway deregulation in cancer development and progression.

Breast cancer is the most common cancer diagnosed among US women and is the second leading cause of cancer-related deaths. TNBC represents 1 of the 4 main molecular subtypes of invasive BC accounting for 10-20% of total cases. It is significantly more common in African American premenopausal women and women with a breast cancer type 1 susceptibility gene (BRCA1) mutation. TNBC is biologically heterogeneous but can be mainly identified by a negative phenotype for the estrogen receptor (ER) and progesterone receptor and a lack of gene amplification/protein overexpression for the human epidermal growth factor receptor 2 (HER2). These biologic characteristics confer a higher aggressiveness and relapse risk than that observed in all other BC subtypes.

Due to the loss of the aforementioned tumor cell receptors, patients with TNBC do not benefit from hormonal therapy or treatments targeting the oncogenic HER2 pathway. The standard of care for patients with recurrent and/or metastatic disease is cytotoxic chemotherapy (taxane- or anthracycline-containing regimens for TNBC, and platinum-based chemotherapy for BRCA1/2 mutation-associated TNBC), leading to a median survival of approximately 13 months from the time of recurrence or diagnosis of distant metastases. A recent meta-analysis of first line treatment in metastatic TNBC in phase 3 studies reported pooled objective response rate (ORR) of 23%, median overall survival (OS) of 17.5 months, and median progression-free survival (PFS) of 5.4 months with single-agent chemotherapy.

Currently, two poly (ADP-ribose) polymerase (PARP) inhibitors olaparib and talazoparib are approved for TNBC patients with BRCA mutations. Atezolizumab in combination with nabpaclitaxel was recently approved for programmed death-ligand 1 (PD-LI)—positive locally advanced or metastatic TNBC.

The Notch pathway is activated during mammary gland development and has been implicated as a key driver in BC. The frequency of Notch mutations or gene rearrangement was reported at 5 to 16% in small cohorts of TNBC tumors and high level of Notch expression was associated with poorer overall survival. In addition, elevated Hes4 expression, a marker of Notch activation, is associated with poorer prognosis in TNBC. In addition, Notch receptors expression and activation strongly correlate with the aggressive clinicopathological and biological phenotypes of BC (e.g., invasiveness and chemoresistance), which are relevant characteristics of TNBC subtype.

In nonclinical models, Compound (1) has a broad-spectrum antitumor activity against solid tumor xenografts of diverse histological types at tolerable doses, including breast carcinoma. Compound (1) exerted its antitumor activity through direct inhibition of cell proliferation and indirectly via inhibition of tumor angiogenesis. In TNBC patient-derived xenograft (PDX) tumor models, the presence of activating Notch mutations/fusions correlated with potent response to Compound (1) monotherapy.

The current study is designed to evaluate the efficacy and safety of Compound (1) monotherapy in subjects with Notch-activated recurrent or metastatic TNBC or Notch-activated endocrine refractory BC; Notch activation will be determined by a Next Generation Sequencing (NGS) test.

TABLE 4
Objectives and Endpoints (Primary and Secondary only)

| | |
|---|---|
| Objectives | Endpoints |

Primary

| | |
|---|---|
| To evaluate the efficacy of Compound (1) | Proportion of subjects who demonstrate an |
| monotherapy in subjects with | overall response rate (ORR) defined as |
| Notch-activated recurrent or metastatic | partial response (PR) + complete response |
| TNBC | (CR) as assessed by investigator based on |
| | Response Evaluation Criteria in Solid |
| | Tumors (RECIST) v1.1 |

Secondary

| | |
|---|---|
| Efficacy - Secondary | |
| | Clinical benefit response rate (CBR) |
| | defined as CR + PR + stable disease (SD) |
| | by investigator review based on |
| | RECIST v1.1 |
| | Duration of response (DOR) by |
| | investigator review based on RECIST v1.1 |
| | Progression free survival (PFS) |
| | Proportion of subjects who have PFS at 6 |
| | months |
| | Overall survival (OS) |
| | Quality of life (QoL) as determined by |
| | European Organization for Research and |
| | Treatment of Cancer Quality of Life |
| | Questionnaire 30 questions (EORTC |
| | QLQ-C30) and Breast Cancer 45 questions |
| | (EORTC QLQ-BR45) |
| To evaluate the safety and tolerability of | |
| Compound (1) monotherapy in subjects | Frequency, duration and severity of |
| with recurrent or metastatic TNBC | treatment-emergent adverse events |
| | (TEAEs) and serious adverse events |
| | (SAEs) |
| | Incidence of clinically significant |
| | abnormalities in laboratory parameters, |
| | ECGs, vital signs and physical |
| | examination |

### Definitions of Efficacy Endpoints

Primary: ORR, defined as the proportion of subjects who have a best overall response (BOR) of CR or PR as determined using RECIST v1.1. BOR is defined as the best response recorded between the date of first dose of IP and the date of subsequent anti-cancer therapy. A BOR of CR or PR requires confirmation of the assessment no earlier than 4 weeks later.

Other efficacy endpoints are as follows:
Progression free is defined as the interval from the start of study treatment to the earlier of the first documentation of disease progression or death from any cause.
Clinical Benefit Response is defined as SD+PR+CR.
DOR, defined as the interval from the first documentation of CR or PR to the earlier of the first documentation of disease progression (per RECIST v1.1) or death from any cause.
OS, defined as the time from the date of start of treatment to the date of death from any cause. Subjects who are lost to follow-up and those not known to have died by the cut-off date for analysis will be censored on the date the subject was last known alive, or the data cut-off date, whichever is earlier.

### Overall Design

This is an open-label multicenter, Phase 2, Simon two-stage optimal design for targeted therapy study of single arm Compound (1) monotherapy in subjects with Notch-activated recurrent or metastatic TNBC or Notch-activated endocrine refractory BC whose disease has recurred or progressed after 3 or fewer lines of prior therapy.

Using the Simon two-stage optimal design (targeting a response of 23% or greater), up to 26 subjects will be enrolled in Stage 1, and 41 additional subjects in Stage 2 (for a total of 67 subjects) provided at least 4 subjects (out of 26) responded in Stage 1. Stage 1 of the Simon two-stage will only include Notch-activated TNBC subjects. Based on results of Stage 1, Stage 2 may also include Notch-activated endocrine refractory BC subjects.

Prior to entering the study, to determine eligibility, potential candidates who sign a separate pre-screening informed consent, will undergo pre-screening assessment for evaluation of Notch mutational status from a persistent locally advanced or metastatic lesion. If historical genotyping results from the last 2 years that identify a Notch-activated genetic alteration are available from a commercially available NGS assay or laboratory developed test (LDT), these results are acceptable to use for determining eligibility. In Europe any commercially available CE marked device can be used.

If acceptable genotyping results are not available, testing of a sample collected from a locally advanced or metastatic lesion must be conducted during pre-screening using a commercially available NGS assay, LDT or other validated investigational use only (IUO) clinical trial assay capable of detecting genetic alterations in the NOTCH 1/2/3/4 genes. For this purpose, tumor biopsy from within the last 2 years or fresh biopsy will be needed.

After signing an informed consent, all subjects will then undergo screening assessments to determine study eligibility over a 28-day Screening period. Eligibility criteria for study entry will be assessed locally by the investigator; radiographic scans at screening and from the prior 12 months will be collected and held for possible future retrospective independent evaluation. Tumor tissue will need to be provided during the Screening period for confirmatory study-specific testing.

Starting on Cycle 1, Day 1, eligible subjects will be treated weekly (QW), on Days 1, 8, and 22 of each 28-day cycle with the investigational product (IP), Compound (1) monotherapy 6 mg intravenously (IV). Treatment will continue until the occurrence of unequivocal radiographic disease progression per RECIST v1.1 as assessed by the investigator, or clear clinical progression as assessed by the investigator, or unacceptable toxicity, or other reasons for discontinuation.

All subjects will receive premedication with H1- and H2-blockers per institution guideline and steroids as prophylaxis. Toxicity post-Compound (1) administration will be managed.

During the Treatment period, subjects will undergo radiographic assessments every 8 weeks (±3 days) for review by the investigator. Radiographic scans will also be collected and held for possible future retrospective independent evaluation. A repeat of tumor imaging will be required for the purposes of confirmation of response (i.e., partial response, and/or complete response). The confirmation scan should be no earlier than 4 weeks following the first indication of response.

Samples from tumor biopsies will be collected at screening from a locally advanced or metastatic lesion (fresh or archival within 2 years), and upon confirmation of disease progression (provided that biopsy collection is medically safe and not contraindicated). If an archival tumor block or 25 unstained slides are not available, the patient will be required to have a fresh tumor sample obtained at screening. Biopsy samples will be evaluated by NGS for genomics, immunohistochemistry (IHC) for Notch intracellular domain (NICD) stain, and other biomarkers potentially related to sensitivity to Compound (1) or TNBC prognosis.

All subjects will undergo end of study (EOS) visit 30 days post last treatment with IP and will be contacted by phone to determine survival status. In subjects who discontinued IP due to toxicity, radiographic imaging will be done every 3 months until disease progression or until the subject initiates another anti-cancer therapy. The end of the study is defined as the date of the last visit of the last subject in the study.

### Rationale for Study Population

The primary target population for this study is subjects with recurrent or metastatic TNBC. This population is selected based on the mechanism of action of Compound (1), nonclinical and preliminary clinical data with Compound (1), as well as the pathophysiology of TNBC. Based on preliminary nonclinical and clinical emerging data, Notch-activated TNBC patient populations are selected to receive Compound (1) as monotherapy.

In TNBC patient-derived xenograft (PDX) tumor models, the presence of an activated Notch pathway signature and Notch mutations/fusions correlates with a significant response to Compound (1) monotherapy. Therefore, the Sponsor will test the hypothesis that subjects diagnosed with TNBC bearing Notch-activated gene alterations responds to Compound (1) monotherapy.

Importantly for the purposes of this study, the definition of TNBC has been evolving over the past few years. According to the 2010 guidelines by the American Society of Clinical Oncology (ASCO) and the College of American Pathologists (CAP), to qualify for TNBC, estrogen receptor (ER)/progesterone receptor positivity was defined as ≤1% positively stained cells by IHC. However, several studies show that tumors with 1%<ER<10% behave similarly to those with ER≤1%. Therefore, it is recommended to define TNBC as human epidermal growth factor receptor 2 (HER2) negative BC with ER and/or progesterone receptor less than 10%. Thus, subjects with less than 10% IHC staining of ER/progesterone receptor will be eligible to participate.

In addition to TNBC patients, there is evidence that a small percentage of endocrine refractory BC subjects also have Notch-activated gene alterations. As a result, this study will also enroll Notch-activated endocrine refractory BC subjects.

### Justification for Dose

The dose selected for this study (6 mg QW) is based on nonclinical studies, prior clinical studies and preliminary outcome from the ongoing ACCURACY study conducted by Ayala (NCT03691207).

In Study CA216001, 94 subjects were treated with Compound (1), of whom 83 subjects received Compound (1) on a weekly (QW) schedule at doses ranging from 0.3 mg to 8.4 mg (with 43 subjects on the 4 mg QW and 14 subjects on the 6 mg QW dosing schedule), and 11 subjects receiving Compound (1) every 2 weeks (Q2W schedule) at doses of 4 mg or 6 mg. Overall, Compound (1) was safe and tolerated in doses up to 4 mg QW administration. The most frequently reported treatment related adverse events (AEs) were diarrhea (62.8% overall; 67.4% in 4 mg QW vs. 71.4% in 6 mg QW dose group), hypophosphatemia (53.2% overall; 60.5% in 4 mg QW vs. 78.6% in 6 mg QW), fatigue (44.7% overall; 34.9% in 4 mg QW vs. 78.6% in 6 mg QW), and nausea (43.6% overall; 41.9% in 4 mg QW vs. 71.4% in 6 mg QW). As dose limiting toxicities (DLTs) were observed in the 6 mg QW (2 cases—Grade 3 vomiting/Grade 3 lipase elevation and Grade 3 diarrhea) and 8.4 mg QW (3 cases—Grade 5 liver failure, recurrent Grade 3 hypersensitivity reaction and Grade 3 vomiting, each of which occurred after 2 doses of Compound (1)) dose levels, the previous developer recommended that the maximum tolerated dose (MTD) for a QW dosing schedule to be 4 mg, and for Q2W dosing schedule to be 6 mg.

PK results from Study CA216001 indicate that Compound (1) exposure (Cₘₐₓ and area under the curve [AUC]) increases approximately linearly for QW dosing from 4 mg to 6 mg. Furthermore, pharmacodynamic results (Hes1 expression in PB) from Study CA216001 indicate greater maximum effect and duration of target inhibition when increasing the QW dose from 4 mg to 6 mg. Mean Hes1 inhibition is maintained greater than 50% throughout a 7 day period at 6 mg, where it recovers to under 50% by day 7 at 4 mg.

In Study CA6216002, 20 subjects with T-ALL and T-LL received Compound (1) 6 mg QW. Overall, this dose was well tolerated in this population, and the expansion cohort was opened at this dose level.

The previous developer decided to discontinue development of Compound (1) due to business reasons and not due to any safety concerns associated with the use of Compound (1).

In the Phase 2 ACCURACY study, preliminary results have been reported. As of Oct. 11, 2019, the safety analysis set included 29 subjects with adenoid cystic carcinoma. The most frequently reported AEs (>30% of all treated subjects), regardless of causality, were nausea, fatigue, diarrhea and vomiting. SAEs were reported for 14 subjects (48.3%) with pneumonia reported by 3 subjects (10.3%), all other events were single incidences. Treatment-related SAEs included infusion site reactions (2 reactions in one subject) and keratoacanthoma (each, 1 subject, 3.4%). The ACCURACY study is ongoing and expanded to include Compound (1) 6 mg QW dosing schedule.

The observed safety profile of Compound (1) in the ACCURACY study to date suggests a lower rate of TEAEs of Grade 3 and above than previously reported for Compound (1) Phase 1 studies conducted by the previous developer. Notably, using the toxicity management guidelines for gastrointestinal (GI) adverse events in this protocol, the rate of Grade 3 diarrhea was reduced to 3.4% vs. 19.1% in the ongoing ACCURACY study vs. study CA216001, respectively. This suggests that with a rigorous control of GI toxicity, a dose of 6 mg is safe to administer to subjects with advanced cancer.

### Disclosure Statement

This is a single group treatment study with no masking.

### Number of Subjects

Using the Simon two-stage optimal design (targeting a response of 23% or greater), up to 26 evaluable Notch-activated TNBC subjects will be enrolled in Stage 1, and 41 additional evaluable subjects (Notch-activated TNBC and possibly endocrine refractory) in Stage 2 (for a total of 67 subjects) provided at least 4 subjects (out of 26) responded in Stage 1. This design allows determination of Compound (1) anti-tumor activity while minimizing the expected sample size.

### Intervention Group

IP: Compound (1) 6 mg IV administered QW, on Days 1, 8, 15 and 22 of each 28-day cycle.

### Study Duration for Each Subject

For each subject, the study is expected to last as follows:

TABLE 5

| | |
|---|---|
| Screening period: | Up to 28 days |
| Treatment period: | Until disease progression, unacceptable toxicity, |
| | or consent withdrawal |
| EOS: | 30 days after the last administration of IP |
| Long term follow-up: | Every 3 months for the first year after EOS, |
| | then every 6 months for the next 2 years and |
| | then annually. |

### Data Monitoring Committee: Yes

Eligibility Criteria

### Inclusion Criteria

To be eligible for participation in this study, subjects must meet all the following criteria: Age
At least 18 years of age (inclusive) at the time of signing the Informed Consent Form (ICF).

### Type of Subject and Disease Characteristics

### All Subjects

1. Have at least one measurable lesion per RECIST v1.1.
2. Have formalin-fixed paraffin-embedded (FFPE) tissue available from a metastatic lesion; a tumor block or 25 unstained slides from an archived (within 2 years) or fresh tumor samples (core or punch needle biopsy) are acceptable.
3. Documented tumor progression following no more than 3 lines of systemic chemotherapy or immunotherapy for metastatic disease. Of note, neoadjuvant and adjuvant therapy will not count as prior lines of therapy.

### TNBC Subjects

4. Histologically confirmed diagnosis of inoperable locally advanced or metastatic TNBC defined as ER and progesterone receptor staining <10%, and HER2-negative defined as IHC 0 to 1+
   Note, if IHC is equivocal then do fluorescence in situ hybridization (FISH) or in situ hybridization (ISH); negative will be acceptable.
   Note: if FISH or ISH is equivocal then further assessment is allowed.
5. Documented Notch activation from tumor biopsy results from within the last 2 years from a commercially available NGS assay, LDT or other validated IUO clinical trial assay.

### Endocrine Refractory Subjects (Stage 2 Only)

6. Histologically confirmed diagnosis of inoperable locally advanced or metastatic hormone receptor positive BC defined as ER and/or progesterone receptor staining ≥10% and HER2-negative as defined in inclusion 7 above.
7. Have prior documented Notch activation from prior tumor biopsy results from a from a commercially available NGS assay, LDT or other validated IUO clinical trial assay.

### Gender and Reproductive Considerations

8. Female or Male subjects.
9. Women of childbearing potential (WOCBP) must have a negative serum or urine pregnancy test (minimum sensitivity 25 IU/L or equivalent units of HCG) within 24 hours prior to the start of IP and must agree to have a pregnancy test at least every cycle (4 weeks). An extension up to 72 hours is permissible in situations where results cannot be obtained within the standard 24-hour window.
Contraception use by men or women should be consistent with local regulations regarding the methods of contraception for those participating in clinical studies.
10. WOCBP must agree to use a highly effective birth control during the study (prior to the first dose with Compound (1) and for 120 days after the last dose), if conception is possible during this interval. Female subjects are considered to not be of childbearing potential if they have a history of hysterectomy or are post-menopausal defined as no menses for 12 months without an alternative medical cause.
11. Male subjects with partners who are WOCBP should use a combination of methods of contraception for the women along with a male condom during the study and for 120 days after the last dose of IP, unless permanently sterile by bilateral orchidectomy.

### Informed Consent

12. Capable of giving signed informed consent form (ICF) which includes compliance with the requirements and restrictions listed in the ICF and in this protocol.

### Exclusion Criteria

The subjects must be excluded from participating in the study if they meet any of the following criteria:

### Medical Conditions

1. A known additional malignancy that is progressing or requires active treatment that is considered medically active and may interfere in the ability to detect responses in this subject. Exceptions include basal cell carcinoma of the skin, squamous cell carcinoma of the skin that have undergone potentially curative therapy or in situ cervical cancer.
2. BC that, in the opinion of the investigator, is considered amenable to potentially curative treatment.
3. Symptomatic central nervous system (CNS) metastases. Subjects with asymptomatic CNS metastases as well as those with previously treated CNS metastases are eligible for enrollment in the study if at least 28 days has elapsed since definitive treatment (either surgery, whole brain radiotherapy, stereotactic radiation), steroid therapy is either not required or dose has been weaned off over last 14 days, and the subject is deemed clinically stable by the investigator.
4. Current or recent (within 2 months of IP administration) gastrointestinal (GI) disease or disorders that increase the risk of diarrhea, such as inflammatory bowel disease and Crohn's disease. Non-chronic conditions (e.g., infectious diarrhea) that are completely resolved for at least 2 weeks prior to starting IP are not exclusionary.
5. Developed immune-mediated colitis with immunotherapy unless resolved to G1 or lower and without requirement of steroid treatment for at least 14 days prior to first dose of IP.
6. Peripheral neuropathy D Grade 2 for at least 14 days prior to first dose of IP.
7. Evidence of uncontrolled, active infection, requiring systemic anti-bacterial, anti-viral or anti-fungal therapy <7 days prior to administration of IP such as known active infection with hepatitis B, hepatitis C, or human immunodeficiency virus (HIV).
8. Unstable or severe uncontrolled medical condition (e.g., unstable cardiac or pulmonary function or uncontrolled diabetes) or any important medical illness or abnormal laboratory finding that would, in the investigator's judgment, increase the risk to the subject associated with his or her participation in the study.
9. Pregnant or breastfeeding or expecting to conceive children within the projected duration of the study.

### Diagnostic Assessments

10. Eastern Cooperative Oncology Group (ECOG) performance status ≥2.
11. Abnormal organ and marrow function defined as:
   a. neutrophils <1000/mm³,
   b. platelet count <75,000/mm³,
   c. hemoglobin <8 g/dL,
   d. total bilirubin >1.5□upper limit of normal (ULN) (except known Gilbert's syndrome),
   e. aspartate aminotransferase (AST) and alanine aminotransferase (ALT) >2.5□ULN OR >5□ULN for subjects with liver metastases,
   f. creatinine clearance (CrCl)<50 mL/min (calculation of CrCl will be based on acceptable institution standard),
   g. uncontrolled triglyceride ≥Grade 2 elevations per CTCAE v5.0 (>300 mg/dL or >3.42 mmol/L).
12. Myocardial infarction within 6 months prior to enrollment or has New York Heart Association (NYHA) Class III or IV heart failure, uncontrolled angina, severe uncontrolled ventricular arrhythmias, or electrocardiographic evidence of acute ischemia or active conduction system abnormalities.
13. Mean QT interval corrected for heart rate using Fridericia's formula (QTcF) ≥480 msec.

### Prior/Concurrent Therapy

14. Completed palliative radiation therapy <7 days prior to initiating IP.
15. Prior treatment with gamma secretase inhibitors. Prior treatment with anti-Notch antibodies may be allowed upon discussion with the Sponsor's medical monitor.
16. Last chemotherapy, biologic, or investigational therapy agent at least 4 weeks or 5 halflives (whichever is shorter) prior to initiating IP; at least 6 weeks if the last regimen included BCNU or mitomycin C. Prior treatment with investigational monoclonal antibody will be reviewed case-by-case by the Sponsor.
17. Receiving chronic systemic steroid therapy (in dosing exceeding 10 mg/day of prednisone or equivalent) or any other form of immunosuppressive therapy within 7 days prior to the first dose of IP. The use of physiologic doses of corticosteroids may be approved after consultation with the Sponsor.
18. Use of strong inhibitors of CYP3A4 within 1 week or 5 half-lives (whichever is longer) or strong inducers of CYP3A4 within 2 weeks or 5 half-lives (whichever is longer).
19. Use of any herbal supplements within 1 week prior to IP administration.
20. Use of medications causing Torsades de Pointes within 1 week or 5 half-lives (whichever is longer).
21. Use of strong inhibitors of CYP3A4 within 1 week or 5 half-lives (whichever is longer) or strong inducers of CYP3A4 within 2 weeks or 5 half-lives (whichever is longer).
22. Subjects who require the use of any of the aforementioned treatments for clinical management should be removed from the study. Subjects may receive other medications that the investigator deems to be medically necessary.
23. The Exclusion Criteria describes other medications that are prohibited in this study.
24. There are no prohibited therapies during the post-treatment follow-up period.

### Other Exclusions

25. Concurrent enrolment in another clinical study, unless it is an observational (noninterventional) clinical study or during the follow-up period of an interventional study.
26. Life expectancy of less than 3 months.
27. Hypersensitivity to Compound (1) and any of its excipients.

### Investigational Product Administered

Compound (1) is a potent and selective inhibitor of gamma secretase-mediated Notch signaling:

TABLE 6

| Intervention Name | Compound (1) |
|---|---|
| Type | Small Molecule |
| Dose | Solution for infusion |
| Formulation | |
| Unit Dose | 1.2 mg/mL |
| Strength(s) | |
| Dosage Level(s) | 6 mg (1.2 mg/mL × 5 mL) |
| | Frequency: Weekly (QW) on Days 1, 8, 15 and 22 of each 28-day cycle |
| Route of | Compound (1) will be administered using an IV infusion pump over 60 |
| Administration | minutes; time windows of −5 minutes to +15 minutes are permitted. The |
| | connection between the tubing from the infusion pump to the subject |
| | should be close to the insertion of the intravenous catheter. |
| | The exact duration of infusion should be recorded in both source |
| | documents and eCRFs. The start time of dose administration will be |
| | called “0” hour. If an infusion is extended, interrupted or discontinued |
| | prior to completion, the duration and the reason for any dose extension, |
| | interruption or discontinuation will be recorded in the eCRF. |
| Preparation | Prior to IV administration, the Compound (1) 6 mg (1.2 mg/mL × 5 mL) |
| | is diluted in a 250 mL infusion bag of 0.9% Sodium Chloride Injection, |
| | USP (normal saline) or 5% Dextrose Injection, USP (D5W). |
| | Only diethylhexyl phthalate-free bags and infusion sets can be used to |
| | administer solutions. Aseptic practices should be followed when |
| | handling, preparing, and administering the infusion solutions because |
| | the drug vial does not contain antibacterial preservatives or |
| | bacteriostatic agents. A sufficient excess of drug product is included in |
| | each vial to account for withdrawal losses. |
| | Refer to Study Pharmacy Manual for more details. |
| IMP definition | A new drug that is used in a clinical investigation (FDA) |
| Sourcing | Compound (1) is manufactured by Bristol-Myers Squibb. |
| | Investigational product will be provided to the site centrally by the |
| | Sponsor or designated representative (Fisher Clinical Services, USA). |
| Packaging and | Compound (1) is supplied as a single-use sterile solution 5 mL per vial |
| Labeling | (1.2 mg/mL; equivalent of 6 mg per vial) for IV administration; The |
| | secondary packaging and labeling of IP will be performed by Fisher |
| | Clinical Services. |
| | All packaging and labeling operations for IP will be performed |
| | according to Good Manufacturing Practices for Medicinal Products and |
| | the relevant regulatory requirements. Label text for the Compound (1) |
| | vial will at a minimum include the protocol number, the contents of the |
| | vial, batch number, storage conditions, and Sponsor name and address. |

### Dose Modification Criteria for Compound (1)

TABLE 7
Dose modification associated with Compound (1) is described in the following table:

| Dose Modification Criteria for | |
|---|---|
| Compound (1) - | |
| Related Adverse Events | Compound (1) modification at next dose |
| Grade 4 neutropenia lasting ≥7 days | Decrease dose to 4 mg for first episode and to 2.4 |
| | mg for second episode. |
| Grade 3 or 4 febrile neutropenia lasting >24 | Decrease dose to 4 mg for first episode and to 2.4 |
| hours | mg for second episode. |
| Grade 4 thrombocytopenia or ≥ Grade 3 | Decrease dose to 4 mg for first episode and to 2.4 |
| thrombocytopenia with significant | mg for second episode |
| bleeding | |
| ≥Grade 2 or higher diarrhea not adequately | Interrupt Compound (1) until resolution to ≤Grade 1. |
| controlled with loperamide at maximum | Decrease dose to 4 mg for first episode and to 2.4 |
| doses or the addition of corticosteroids | mg for second episode. |
| | If treatment interruption is longer than 28 days, |
| | the subject will be discontinued from the study. |
| Diagnosed or suspected clinically | Interrupt Compound (1), perform appropriate |
| significant | diagnosis and treatment. |
| GI bleeding or unexplained drop in | If GI bleeding is considered related to Compound |
| hemoglobin | (1), Decrease to 4 mg for first episode and to 2.4 |
| | mg for second episode 1 dose level. |
| | If treatment interruption is longer than 28 days, |
| | the subject will be discontinued from the study. |
| QTcF >500 msec confirmed by at least | Interrupt if needed to optimize electrolyte |
| one repeat ECG and at least 50 msec above | management. If persists after electrolyte |
| baseline | optimization (including dose modification of |
| | Compound (1) if necessary), discontinue. |
| | If treatment interruption is longer than 28 days, |
| | the subject will be discontinued from the study. |
| AST or ALT >5 times the institutional | Interrupt Compound (1) until resolution to ≤Grade 1 |
| ULN | and then decrease dose to 4 mg for first |
| | episode and to 2.4 mg for second episode. |
| | If treatment interruption is longer than 28 days, |
| | the subject will be discontinued from the study. |
| Hy's law cases (e.g. drug induced liver | Immediately discontinue |
| injury; DILI) | |
| Triglyceride Grade 3 elevations that persist | Interrupt Compound (1) until resolution to ≤Grade 1 |
| after 4 weeks of medical management | and then decrease dose to 4 mg for first |
| | episode and to 2.4 mg for second episode. |
| | If treatment interruption is longer than 28 days, |
| | the subject will be discontinued from the study. |
| Infusion-related reaction | For dose modification and management of |
| | infusion-related reactions |
| Any other Compound (1)-related ≥Grade 3 | Interrupt Compound (1) until resolution to ≤Grade 1 |
| nonhematologic adverse event except | and then decrease dose to 4 mg for first |
| electrolyte abnormalities that may be | episode and to 2.4 mg for second episode. |
| managed with supplements | If treatment interruption is longer than 28 days, |
| | the subject will be discontinued from the study. |

### Toxicity Management Guidelines

### Treatment of Infusion Reactions

In case of hypersensitivity reactions, the investigator should institute treatment measures deemed medically appropriate in accordance with current medical practice and treatment guidelines.
Grade 1 allergic reaction/hypersensitivity (e.g., transient flushing, rash, drug fever <38° C.):
   [symbol] Supervise at the bedside.
Grade 2 allergic reaction/hypersensitivity (e.g., urticaria, drug fever □38° C., rash, flushing, dyspnea):
   [symbol] Interrupt the infusion and disconnect infusion tubing from subject,
   [symbol] Administer IV antihistamines (diphenhydramine 25 to 50 mg and famotidine 20 to 40 mg or class equivalents),
   [symbol] After recovery from symptoms, resume the infusion at a half of the infusion rate and if no further symptoms appear, complete the administration of the dose. A target infusion time of up to 3 hours may be appropriate in some cases.
Grade 3 or 4 allergic reaction/hypersensitivity (e.g., symptomatic bronchospasm requiring parenteral medication(s) with or without urticaria; allergy-related edema/angioedema; anaphylaxis; hypotension):
   Stop the infusion with Compound (1) and disconnect infusion tubing from subject,
   [symbol] Administer epinephrine, antihistamines, and nebulized bronchodilators as medically indicated,
   [symbol] Consider IV steroids which may prevent recurrent or ongoing reactions,
   [symbol] Report as a serious adverse event,
   [symbol] Discontinue the subject from the study for a Grade 4 or for a second episode of a Grade 3 reaction.

Other symptoms associated with hypersensitivity reactions include facial flushing, chest pain and tightness, back pain and GI symptoms, leg pain and cough.

Retreatment after a Grade 3 or greater hypersensitivity reaction despite premedication should be discussed between the Sponsor's Medical Monitor and investigator prior to retreatment. If treatment interruption is longer than 28 days, the subject will be discontinued from the study.

### Guidelines for Management of Diarrhea

The following are guidelines for the management of diarrhea and are not meant to replace the clinical judgment of the investigator/treating physician(s) or an institutional diarrhea management protocol which adheres to most current medical standards.

1. Treat with Loperamide

Loperamide should be started at the earliest sign of (1) a poorly formed or loose stool, (2) the occurrence of 1 to 2 more bowel movements than usual in 1 day, or (3) an increase in stool volume or liquidity. Loperamide may be taken in the following manners: 4 mg at the first onset of diarrhea, then 2 mg every 2 hours around-the-clock until diarrhea free for at least 12 hours. Subjects may take loperamide 4 mg every 4 hours during the night. This dosing regimen is higher than the standard dose of loperamide, but is typical for the treatment of diarrhea caused by anticancer therapy. These doses should not be used for more than 48 hours due to the risk of paralytic ileus. Subjects should be provided with loperamide at the initial treatment visit so that they have sufficient supply on hand in case antidiarrheal support is required. It is important that loperamide is taken as instructed, as some cases of higher-grade diarrhea have occurred in subjects not taking the maximum doses, and these cases have improved after loperamide was taken more frequently. For subjects that cannot tolerate loperamide or do not get adequate relief with maximum doses, standard doses of LOMOTIL® (diphenoxylate/atropine) may be added or used instead of loperamide. Additional antidiarrheal measures, such as octreotide, may be used at the discretion of the investigator or treating physician.

2. Treat with Dexamethasone

For Grade 2 or higher diarrhea that is not adequately controlled with loperamide and dose interruption, administration of corticosteroid may be considered at the discretion of the treating physician/investigator.

3. Interrupt AL 101 Dosing

For Grade 2 or higher diarrhea that is not controlled (i.e., to Grade 1) with loperamide, dosing of Compound (1) should be interrupted, as continued dosing is likely to result in increased severity of diarrhea. In addition, evaluation of infectious causes should be considered. Based on the mechanism of action and preliminary clinical experience, full gastrointestinal (GI) tract recovery will likely take longer than the time for diarrhea to resolve. Thus, interruption for 5 days to 7 days beyond the resolution of diarrhea should be considered. Depending on the severity, time to onset, and time to resolution of diarrhea, reduction of Compound (1) dose and/or frequency, omission of future doses, or corticosteroid co-administration should be considered.

4. Increase Fluid Intake and, if Applicable, Consider Stopping Antihypertensive Therapy and Nonsteroidal Anti-Inflammatory Drugs

Hypotension and/or renal insufficiency can occur in the setting of volume depletion from severe diarrhea. At the onset of any diarrhea, subjects should be instructed to increase fluid intake to help maintain fluid and electrolyte balance during episodes of diarrhea. Parenteral hydration should be started if oral hydration is not sufficient. The investigator should consider interrupting antihypertensive therapy and nonsteroidal anti-inflammatory drugs, if medically appropriate.

### Guidelines for the Management of Hepatotoxicity

Hepatic function abnormality is defined as any increase in ALT or AST to greater than 3×ULN and concurrent increase in total bilirubin to be greater than 2×ULN. Concurrent findings are those that derive from a single blood draw or from separate blood draws taken within 8 days of each other. Follow-up investigations and inquiries will be initiated promptly by the investigational site to determine whether the findings are reproducible and/or whether there is objective evidence that clearly supports causation by a disease (e.g., cholelithiasis and bile duct obstruction with distended gallbladder) or an agent other than the IP.

Cases where a subject shows an AST or ALT≥3□ULN and total bilirubin ≥2×ULN may need to be reported as SAEs. These cases should be reported as SAEs if, after evaluation they meet the criteria for a Hy's Law case or if any of the individual liver test parameters fulfill any of the SAE criteria. IP should be interrupted immediately if any Hy's law cases.

### Potential Drug-Induced Liver Injury (DILI)/Hy's Law

Wherever possible, timely confirmation of initial liver-related laboratory abnormalities should occur prior to the reporting of a potential DILI event. All occurrences of potential DILIs, meeting the defined criteria, must be reported as SAEs. The criteria for identifying p-DILI events depend on whether the subject's baseline liver biochemistry is normal or abnormal.

### Guidelines for the Management of Colitis

TABLE 8
Guidelines for the management of colitis are as follows:

| | Dose | |
|---|---|---|
| Grade | Modification | Management |
| Any | | Subjects should be thoroughly evaluated to rule out any |
| Grade | | alternative etiology (e.g., disease progression, other |
| | | medications, infections including testing for Clostridium |
| | | difficile toxin, etc.) |
| | | Steroids should be considered in the absence of clear |
| | | alternative etiology, even for low grade events, in order |
| | | to prevent potential progression to higher grade event |
| | | Use analgesics carefully; they can mask symptoms of |
| | | perforation and peritonitis |
| Grade 1 | No dose | Actively monitor frequency, consistency and appearance of |
| | modification | stools (especially for presence of mucus or blood) and for the |
| | | emergence of abdominal pain or cramps |
| Grade 2 | Decrease dose by | Promptly start prednisone 1 to 2 mg/kg/day or IV |
| | 40% to a final dose | equivalent |
| | of 2.4 mg | If event is not responsive within 3-5 days or worsens |
| | | despite prednisone at 1-2 mg/kg/day or IV equivalent, |
| | | GI consult should be obtained for consideration of |
| | | further workup such as imaging and/or colonoscopy to |
| | | confirm colitis and rule out perforation or other cause |
| | | such as |
| | | C. difficle colitis |
| | | Prompt treat with IV methylprednisolone 2- |
| | | 4 mg/kg/day |
| | | started or, for C. difficile, treat with appropriate |
| | | antibiotics. |
| | | Caution: Important to rule out bowel perforation |
| | | Consult study physician if no resolution to ≤Grade 1 in |
| | | 3-4 days |
| | | Once improving, gradually taper steroids over ≥28 days and |
| | | consider prophylactic antibiotics, antifungals and anti PCP |
| | | treatment (please refer to current NCCN guidelines for |
| | | treatment of cancer-related infections [Category 2B |
| | | recommendation]) |
| Grade 3-4 | Permanently | Promptly initiate empiric IV methylprednisolone 2 to 4 |
| | discontinue IP | mg/kg/day or equivalent |
| | | Monitor stool frequency and volume and maintain |
| | | hydration |
| | | Urgent GI consult and imaging and/or colonoscopy as |
| | | appropriate |
| | | Caution: Ensure GI consult to rule out bowel perforation |
| | | Once improving, gradually taper steroids over ≥28 days and |
| | | consider prophylactic antibiotics, antifungals and anti PCP |
| | | treatment (please refer to current National Comprehensive |
| | | Cancer Network [NCCN] guidelines for treatment of cancer- |
| | | related infections [Category 2B recommendation]) |

### Concomitant Therapy

Concomitant medication is defined as any prescription or over-the-counter preparation, including vitamins and supplements. Use of concomitant medication from 28 days before Day 1 of Cycle 1 through 30 days after the last dose of IP must be recorded onto the eCRF from the subject's medical file. This will include trade name or generic name, strength, unit, route of administration, dosage form, frequency, indication, start and stop date(s) of administration.

### Premedication to Prevent Hypersensitivity Reaction

Histamine is a major mediator of anaphylactic/anaphylactoid responses in man, such as those induced by Cremophor EL, an excipient in Compound (1). The premedication regimen below is based on clinical experience with other compounds containing Cremophor EL. In order to prevent a hypersensitivity reaction, all subjects initiating Compound (1) treatment will be premedicated approximately 1 hour prior to the infusion of Compound (1) with the following regimen:

H1-blocker (for example, diphenhydramine 25 to 50 mg oral or equivalent), and H2-blocker (for example, famotidine 20 to 40 mg oral or equivalent).

For subjects who remain on study for more than 4 doses of Compound (1) without any evidence of infusion-related reaction, modification of the premedication regimen may be considered at the discretion of the investigator, with notification of the Sponsor's Medical Monitor. At this time, 1 of the 2 histamine blockers may be discontinued; if there is still no evidence of infusion-related reaction with the next 2 doses of Compound (1), the other may be discontinued. If under this discontinuation plan, the subject has an infusion-related reaction resulting in medical treatment, premedication with H1- and/or H2-blockers (as appropriate) should be resumed for subsequent doses.

If a subject experiences a Grade 3 or 4 infusion-related reaction despite pretreatment with the H1- and H2-blockers then the subject, if re-treated, should also be premedicated with corticosteroids in addition to the H1- and H2-blockers). In the event that a subject has a repeat Grade 3 or 4 infusion-related reaction despite premedication with H1- and H2-blockers and steroid, the subject must not receive any further treatment with Compound (1), unless agreed by the Sponsor/Medical Monitor and investigator that it is in the subject's best interest to continue treatment (e.g. subject has had a response to therapy) and appropriate safety measures can be implemented. Such measures may include dose reduction, increased infusion time, initial lower infusion rate with gradual increases, and/or premedication with multiple doses of dexamethasone. These measures have been used to allow re-treatment after infusion reactions with other agents, including IXEMPRA and Taxol.

Premedication with Corticosteroids

All subjects will receive premedication with corticosteroids as prophylaxis utilizing the following regimen.
8 mg dexamethasone Per Os (PO) the night before each infusion
8 mg dexamethasone PO or IV within 30 minutes prior to dosing
8 mg PO every 8 hours for an additional 4 doses starting about 4 to 8 hours after the infusion is finished
Repeat for the first 4 doses (first cycle).
   If there are no GI toxicities following the first 4 infusions, the number of additional doses following the future infusions can be decreased from 4 to 2 doses of 8 mg PO every 8 hours.

Further tapering should only be considered if, in the opinion of the investigator, steroid side effects are an issue and after discussion with the Sponsor's Medical Monitor.

Other steroids, such as budesonide or prednisone may be used utilizing “prednisone equivalent” conversions.

### Allowed Medications

All treatments that the investigator considers necessary for a subject's welfare may be administered at the discretion of the investigator in keeping with the community standards of medical care. All concomitant medication will be recorded on the eCRF including all prescription, over-the-counter (OTC), herbal supplements, and IV medications and fluids. If changes occur during the study period, documentation of drug dosage, frequency, route, and date may also be included on the eCRF.

All concomitant medications received within 28 days before the first dose of IP and 30 days after the last dose of IP should be recorded. Concomitant medications administered after 30 days after the last dose of IP should be recorded for SAEs and Adverse events of special interest (AESIs).

In addition, all prior therapies for BC should be recorded.

Allowed concomitant therapies:
Glucocorticoids.
In case of toxicity (e.g., GI AEs), per discretion of the investigator and in consultation with the Sponsor's Medical Monitor, dexamethasone (for example
4-8 mg every 6 hours for up to 72 hours, starting 12 hours before Compound (1) administration or per institution guidelines) will be permitted.
Palliative radiation therapy to a limited field (e.g., painful bone metastasis, painful lumps), if it is not the sole site of measurable and/or assessable disease, is allowed any time during study participation with prior approval of the Sponsor's Medical Monitor.

### Discontinuation of Investigational Product

In some instances, it may be necessary for a subject to permanently discontinue IP.

Dosing of IP must be interrupted for any serious adverse reactions assessed by the investigator as related to IP.

Discontinuation of IP does not represent withdrawal from the study. Subjects may withdraw or be withdrawn from IP at any time.

A subject may discontinue IP for reasons including but not limited to:
Adverse event
Death
Lost to follow-up
Non-compliance with study drug
Physician decision
Pregnancy
Progressive disease
Protocol deviation
Study terminated by Sponsor
Withdrawal by subject

### Efficacy

### Efficacy Assessments

### Response Evaluation Criteria in Solid Tumors (RECIST) v1.1

The primary efficacy endpoint, ORR, will be evaluated using RECIST v1.1 (Eisenhauer, 2009, incorporated herein by refernece). A repeat of tumor imaging will be required for the purposes of confirmation of response (i.e., partial response, and/or complete response). The confirmation scan should be no earlier than 4 weeks following the first indication of response. Imaging guidelines are as follows:

TABLE 9

| Study Period | Schedule | Imaging (RECIST 1.1) | |
|---|---|---|---|
| Screening | Within 28 days prior | At screening, the following imaging scans are | |
| | to day 1 | required: | |
| | | 1. | Brain MRI (preferred) or CT (acceptable) with |
| | | | contrast |
| | | 2. | CT of chest |
| | | 3. | CT of abdomen |
| | | 4. | CT of pelvis |
| | | 5. | Bone scan |
| Treatment | Every 8 weeks (±3 | Follow-up scans at 8-week intervals, the following | |
| | days) until disease | image scans are required: | |
| | progression | 1. | Brain MRI/CT with contrast for known or |
| | | | suspected disease, can be done every 12 weeks; |
| | | | same modality as screening should be used |
| | | | throughout the study. |
| | | 2. | CT of chest |
| | | 3. | CT of abdomen |
| | | 4. | CT of pelvis |
| | | 5. | Bone scan to be done every 16 weeks, if bone |
| | | | metastases at Screening |
| Early | 30 days post last IP | At Early Discontinuation or EOS, the following image | |
| discontinuation | (±7 days) | scans are required: | |
| or EOS Visit | | 1. | Brain MRI/CT with contrast for known or |
| | | | suspected disease; same modality throughout the |
| | | | study. |
| | | 2. | CT of chest |
| | | 3. | CT of abdomen |
| | | 4. | CT of pelvis |
| | | 5. | Bone scan, if indicated for known or suspected |
| | | | disease |
| Long-Term | Every 3 months (±7 | Only in subjects who discontinued IP due to toxicity, | |
| Follow-up³ | days) until disease | the following Long-Term Follow-up image scans are | |
| | progression or until | required: | |
| | the subject initiates | 1. | Brain MRI/CT with contrast for known or |
| | another anti-cancer | | suspected disease; same modality throughout the |
| | therapy | | study. |
| | | 2. | CT of chest |
| | | 3. | CT of abdomen |
| | | 4. | CT of pelvis |
| | | 5. | Bone scan, if indicated for known or suspected |
| | | | disease |
| Confirmation | The confirmation | For confirmation no earlier than 4 weeks following a | |
| of Response | scan should be no | PR or CR. the following are required: | |
| Scan | earlier than 4 weeks | 1. | Brain MRI/CT with contrast for known or |
| | following first | | suspected disease; same modality throughout the |
| | indication of | | study. |
| | response | 2. | CT of chest |
| | (PR or CR) | 3. | CT of abdomen |
| | | 4. | CT of pelvis |
| | | 5. | Bone scan, if indicated for known or suspected |
| | | | disease |

### Patient Reported Outcome: European Organization for Research and Treatment of Cancer Quality of Life Questionnaire 30 Questions (EORTC QLQ-C30) and Breast Cancer 45 Questions (EORTC QLQ-BR45)

The EORTC QLQ-C30 and EORTC breast cancer module QLQ-BR45 will be administered during the study every 4 weeks before IP administration.

EORTC QLQ-C30 was developed as an instrument to measure cancer subjects' physical, psychological and social functions (Kaasa, 1995, Eur J Cancer 31A(13-14): 2260-2263, incorporated herein by reference). The questionnaire is composed of 5 multi-item scales (physical, role, social, emotional and cognitive functioning) and 9 single items (pain, fatigue, financial impact, appetite loss, nausea/vomiting, diarrhea, constipation, sleep disturbance and quality of life). It is validated and reliable and has been used successfully in various types of cancer, including BC.

EORTC QLQ-BR23 was developed as an add-on instrument to EORTC QLQ-C30, to measure specifically measure breast cancer quality of life using 23 items. Recently, it was updated to include 45 items (EORTC QLQ-BR45) to include 23 items from the QLQ-BR23 and 22 new items (Bjelic-Radisic, 2018 Annals of Oncology 29(suppl_8), incorporated herein by reference). The new items contain two multi-item scales: a target symptom scale and a satisfaction scale. The target symptom scale can be divided into three subscales: endocrine therapy, endocrine sexual and skin/mucosa scale (Bjelic-Radisic, 2020m Ann Oncol 31(2): 283-288, incorporated herein by reference). The new EORTC QLQ-BR45 module that provides a more accurate and comprehensive assessment of the impact of new and scalable treatments on patients' QoL.

### Safety

### Safety and Tolerability Analysis

All safety analyses will be made on the Safety Analysis population.

The safety assessment will be based on the frequency of AEs, the incidence of clinically significant abnormalities of laboratory values, concomitant medication use, vital signs, pain assessment and physical examination data.

### Safety Assessments

Planned time points for all safety assessments are provided in the Schedule of Activities.

### Medical History and Prior Therapies

Any clinically significant diseases in the prior 3 years including any co-morbid conditions requiring active treatment as well as significant surgeries will be documented. This includes prior medical history and treatment regimen for TNBC.

Abnormal physical examination finding and/or the diagnosis of concomitant disease resulting from assessment at Screening must be also documented in the medical history section. Information on all interventions (systemic therapy, surgery, radiation treatment) related to the subject's cancer will also be collected. Radiology and photography reports from imaging conducted as routine care will be collected if available from the last 3 years.

### Eastern Cooperative Oncology Group (ECOG) Performance Status

The Eastern Cooperative Oncology Group (ECOG) Performance Status will be used to assess subjects' performance status (see following table).

TABLE 10
ECOG PERFORMANCE STATUS

| Grade | ECOG |
|---|---|
| 0 | Fully active, able to carry on all pre-disease performance without restriction |
| 1 | Restricted in physically strenuous activity but ambulatory and able to carry out work of |
| | a light or sedentary nature, e.g., light housework, office work |
| 2 | Ambulatory and capable of all self-care but unable to carry out any work activities. Up |
| | and about more than 50% of waking hours |
| 3 | Capable of only limited self-care, confined to bed or chair more than 50% of waking |
| | hours |
| 4 | Completely disabled. Cannot carry on any self-care. Totally confined to bed or chair |
| 5 | Dead |

### 1. Physical Examination

The complete physical examination will include appearance, eyes, ears, nose, head, throat, neck, lungs, heart, abdomen, extremities, skin, and musculoskeletal system.

Treatment directed physical examination.

### 2. Vital Signs

Vital signs (to be taken before blood collection for laboratory tests) will be measured at all study visits and will include heart rate, respiratory rate, temperature, and blood pressure (systolic and diastolic). Blood pressure and heart rate will be done at rest as per standard practice at the investigational site.

### 3. Electrocardiogram

Single 12-lead ECG will be obtained and evaluated locally. Additional timepoints may be added, as clinically indicated.

Subjects should be in the supine position after the subject has rested for at least 5 minutes. In the event of possible ECG findings, additional ECG reads could be added at follow-up visits.

### 4. Clinical Safety Laboratory Assessments

Clinical laboratory tests to be performed according to the Schedule of Activities.

### Adverse Events of Special Interest

AESI observed with Compound (1) are detailed in the sections below and include hepatic function abnormalities, colitis, anaphylaxis, keratoacanthoma, and hepatic toxicities including drug induced liver injury (DILI).

### 1. Colitis

In study CA216001, DLTs consisting of Grade 3 colonic ulceration/Grade 3 diarrhea were reported, both indicating the potential for development of colitis. Intestinal inflammation is thought to be an on-target effect that requires close monitoring and potential dose reductions. Intense abdominal pain, severe diarrhea and the presence of blood and/or mucous in the stools are indicative of potential colitis. Signs and symptom of colitis should prompt work up to rule out an infectious etiology. The gold standard for the diagnosis of colitis pathological and thus requires a biopsy, but in the absence of an infectious etiology, colitis should be the exclusion diagnosis.

### 2. Anaphylaxis

In study CA216001 1 subject developed G3 anaphylaxis, a DLT at the 4 mg dose.

### 3. Keratoacanthoma

Two cases of keratoacanthoma (a well-differentiated variant of SCC, sometimes considered benign) were reported with Compound (1) (4 mg QW):
In the BMS study CA216001, a Grade 2 keratoacanthoma (subject 3-37) was assessed as related Compound (1) and occurred 3-5 months after initiation of Compound (1).
In the ongoing AL-ACC-01 (ACCURACY) study, a Grade 1 keratoacanthoma (subject no. 1101-002), was assessed by both the investigator and the Sponsor as related to Compound (1) and unexpected.

All subjects should be closely monitored for skin changes by the investigators throughout the study. Any changes suspicious for malignancy should be evaluated by a dermatologist and treated appropriately. In addition, to remove additional risk factors for developing keratoacanthoma, all subjects will be counseled to avoid excessive sun and UV exposure during the study.

### 4. Hepatic Function Abnormalities (Hepatotoxicity)

In study CA216001 a G5 (fatal) case of liver toxicity was reported at the 8.4 mg dose in the escalation phase of the study (refer to the Investigator's Brochure).

Hepatic function abnormality is defined as any increase in ALT or AST to greater than 3×ULN and concurrent increase in total bilirubin to be greater than 2×ULN. Concurrent findings are those that derive from a single blood draw or from separate blood draws taken within 8 days of each other.

Cases where a subject shows an AST or ALT≥3□ULN and total bilirubin ≥2×ULN may need to be reported as SAEs. These cases should be reported as SAEs if, after evaluation they meet the criteria for a Hy's Law case or if any of the individual liver test parameters fulfill any of the SAE criteria.

### Biomarkers

### Activating Notch Alteration Detection by NGS Assay

The planned use of a commercially available NGS assay, LDT or other validated IUO clinical trial assay capable of detecting activating genetic alterations in the NOTCH 1/2/3/4 genes is to identify for inclusion those subjects most likely to benefit from therapy with Compound (1). Notch mutational status from prior tests with commercially available or LDT NGS assay as assessed at enrollment may be confirmed centrally. In the US, any FDA-cleared/approved, validated CTA, or LDT NGS assay is acceptable. In Europe any commercially available CE marked device can be used.

### Other Biomarkers

Predictive biomarkers of response or resistance to the IP will be explored, such as but not limited to:
IHC: Tumor specimens will be stained for the NICD and potentially other biomarkers.
NGS: Mutational analysis will be performed in tumor tissue samples and potentially in blood (cfDNA, CTCs).

Biomarkers indicative of drug activity may be explored, such as but not limited to:
CTC enumeration.
Expression of pharmacodynamic biomarkers of Notch inhibition in tumor tissue and/or CTCs.

Samples may be stored according to local regulations following the last subject's last visit for the study to enable further analysis of biomarker responses to Compound (1).

### Sample Collection

Tumor tissue and blood will be collected at the times designated in the Schedule of Activities. Tumor tissue for biomarker analysis will be required for study participation at screening (block or 25 unstained slides). Tumor biopsies will also be taken at disease progression (end of study). Screening biopsies may be fresh, or archival from within 2 years. Progression biopsies do not need to be done if no tumor is accessible for biopsy or that biopsy poses too great of a risk to the subject. (If the only tumor accessible for biopsy is also the only lesion that can be used for RECIST v1.1 response evaluation, then the subject may be exempt from biopsy).

### Stipulations for Tumor Biopsy

Subjects provide 2 separate tumor biopsies, at screening and progression (end of study). Screening biopsies may be fresh, or archival from within 2 years. Progression biopsies do not need to be done if either investigator or person performing the biopsy judges that no tumor is accessible for biopsy or that biopsy poses too great of a risk to the subject. (If the only tumor accessible for biopsy is also the only lesion that can be used for RECIST v1.1 response evaluation, then the subject may be exempt from biopsy).

### Benefit Assessment

Nonclinical and clinical data provide rationale for evaluating the potential clinical benefits of Compound (1) in subjects with TNBC for whom available standard of care is not providing durable response as defined by complete (CR) or partial response (PR). In a Phase 1 study (CA216003, NCT01653470), clinical activity of Compound (1) was shown in an unselected heavily treated patient population with solid tumors; confirmed objective responses were reported for 8 of the 22 subjects (36.4%) with TNBC who were treated with Compound (1) in combination with various chemotherapy agents (1 subject with a CR and 7 subjects with PR). It is estimated that treatment with Compound (1) may have a positive impact in patients with Notch-activated recurrent or metastatic TNBC, who may thus derive benefit from this treatment.

1. A method of reducing tumor size, suppressing tumor growth, or inhibiting tumor growth in a subject having breast cancer characterized by an activated Notch pathway, comprising the step of administering to said subject a composition comprising one or more compounds represented by the structure of Formula (III): 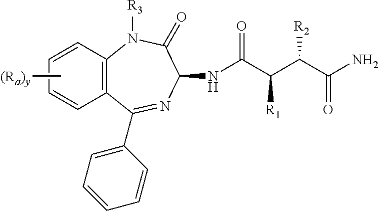
or prodrugs or salts thereof; wherein:
R₁ is —CH₂CF₃ or —CH₂CH₂CF₃;
R₂ is —CH₂CF₃, —CH₂CH₂CF₃, or —CH₂CH₂CH₂CF₃;
R₃ is H or —CH₃;
each Rₐ is independently F, Cl, —CN, —OCH₃, and/or —NHCH₂CH₂OCH₃; and
y is zero, 1, or 2.

2. (canceled)

3. The method of claim 1, wherein:
R₁ is —CH₂CF₃ or —CH₂CH₂CF₃; and
R₂ is —CH₂CF₃ or —CH₂CH₂CF₃.

4. The method of claim 1, wherein:
y is zero or 1.

5.-6. (canceled)

7. The method of claim 1, wherein said compound comprises: (2R,3S)-N-((3S)-1-Methyl-2-oxo-5-phenyl-2,3-dihydro-1H-1,4-benzodiazepin-3-yl)-2,3-bis(3,3,3-trifluoropropyl)succinamide (1); (2R,3S)-N-((3S)-2-Oxo-5-phenyl-2,3-dihydro-1H-1,4-benzodiazepin-3-yl)-2,3-bis(3,3,3-trifluoropropyl)succinamide (2); (2R,3S)-N-((3S)-1-Methyl-2-oxo-5-phenyl-2,3-dihydro-1H-1,4-benzodiazepin-3-yl)-2-(2,2,2-trifluoroethyl)-3-(3,3,3-trifluoropropyl)succinamide (3); (2R,3S)-N-((3S)-1-Methyl-2-oxo-5-phenyl-2,3-dihydro-1H-1,4-benzodiazepin-3-yl)-3-(2,2,2-trifluoroethyl)-2-(3,3,3-trifluoropropyl)succinamide (4); (2R,3S)-N-((3S)-1-(2H3)Methyl-2-oxo-5-phenyl-2,3-dihydro-1H-1,4-benzodiazepin-3-yl)-2,3-bis(3,3,3-trifluoropropyl)succinamide (5); (2R,3S)-N-((3S)-7-chloro-1-methyl-2-oxo-5-phenyl-2,3-dihydro-1H-1,4-benzodiazepin-3-yl)-2,3-bis(3,3,3-trifluoropropyl)succinamide (6); (2R,3S)-N-((3S)-8-methoxy-1-methyl-2-oxo-5-phenyl-2,3-dihydro-1H-1,4-benzodiazepin-3-yl)-2,3-bis(3,3,3-trifluoropropyl)succinamide (7); (2R,3S)-N-((3S)-8-fluoro-1-methyl-2-oxo-5-phenyl-2,3-dihydro-1H-1,4-benzodiazepin-3-yl)-2,3-bis(3,3,3-trifluoropropyl)succinamide (8); (2R,3S)-N-((3S)-7-methoxy-1-methyl-2-oxo-5-phenyl-2,3-dihydro-1H-1,4-benzodiazepin-3-yl)-2,3-bis(3,3,3-trifluoropropyl)succinamide (9); (2R,3S)-N-((3S)-7-fluoro-1-methyl-2-oxo-5-phenyl-2,3-dihydro-1H-1,4-benzodiazepin-3-yl)-2,3-bis(3,3,3-trifluoropropyl)succinamide (10); (2R,3S)-N-((3S)-8-chloro-1-methyl-2-oxo-5-phenyl-2,3-dihydro-1H-1,4-benzodiazepin-3-yl)-2,3-bis(3,3,3-trifluoropropyl)succinamide (11); (2R,3S)-N-((3S)-9-methoxy-2-oxo-5-phenyl-2,3-dihydro-1H-1,4-benzodiazepin-3-yl)-2,3-bis(3,3,3-trifluoropropyl)succinamide (12); (2R,3S)-N-((3S)-8-methoxy-2-oxo-5-phenyl-2,3-dihydro-1H-1,4-benzodiazepin-3-yl)-2,3-bis(3,3,3-trifluoropropyl)succinamide (13); (2R,3S)-N-((3S)-7-methoxy-2-oxo-5-phenyl-2,3-dihydro-1H-1,4-benzodiazepin-3-yl)-2,3-bis(3,3,3-trifluoropropyl)succinamide (14); (2R,3S)-N-((3S)-8-cyano-9-methoxy-2-oxo-5-phenyl-2,3-dihydro-1H-1,4-benzodiazepin-3-yl)-2,3-bis(3,3,3-trifluoropropyl)succinamide (15); (2R,3S)-N-((3S)-8,9-dichloro-2-oxo-5-phenyl-2,3-dihydro-1H-1,4-benzodiazepin-3-yl)-2,3-bis(3,3,3-trifluoropropyl)succinamide (16); (2R,3S)-N-((3S)-9-fluoro-2-oxo-5-phenyl-2,3-dihydro-1H-1,4-benzodiazepin-3-yl)-2,3-bis(3,3,3-trifluoropropyl)succinamide (17); (2R,3S)-N-((3S)-9-chloro-2-oxo-5-phenyl-2,3-dihydro-1H-1,4-benzodiazepin-3-yl)-2,3-bis(3,3,3-trifluoropropyl)succinamide (18); (2R,3S)-N-((3S)-2-oxo-5-phenyl-2,3-dihydro-1H-1,4-benzodiazepin-3-yl)-3-(4,4,4-trifluorobutyl)-2-(3,3,3-trifluoropropyl)succinamide (19); (2R,3S)N-((3S)-8-Methoxy-2-oxo-5-phenyl-2,3-dihydro-1H-1,4-benzodiazepin-3-yl)-3-(4,4,4-trifluorobutyl)-2-(3,3,3-trifluoropropyl)succinamide (20); or (2R,3S)-N-((3S)-9-((2-Methoxyethyl)amino)-2-oxo-5-phenyl-2,3-dihydro-1H-1,4-benzodiazepin-3-yl)-2,3-bis(3,3,3-trifluoropropyl)succinamide (21).

8. The method of claim 1, wherein said compound comprises:

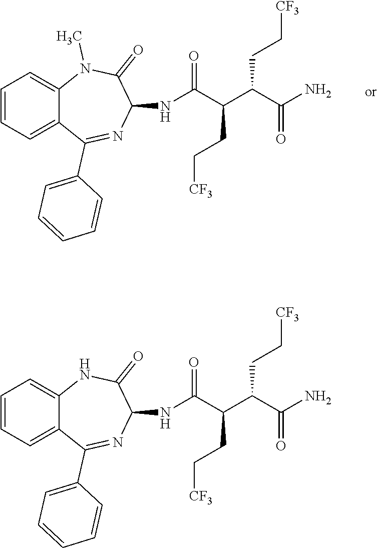 [SMILES: CN1C(=O)[C@@H](NC(=O)[C@H](CCC(F)(F)F)[C@H](CCC(F)(F)F)C(N)=O)N=C(c2ccccc2)c2ccccc21 ; NC(=O)[C@@H](CCC(F)(F)F)[C@@H](CCC(F)(F)F)C(=O)N[C@H]1N=C(c2ccccc2)c2ccccc2NC1=O]

9. (canceled)

10. The method of claim 1, wherein said breast cancer comprises triple negative breast cancer (TNBC).

11. The method of claim 10, wherein TNBC comprises luminal androgen receptor (LAR) TNBC, basal-like TNBC, immunomodulatory (IM) TNBC, mesenchymal (M) TNBC, mesenchymal stem like (MSL) TNBC, or unstable (UNS) TNBC.

12. The method of claim 11, wherein said basal-like TNBC comprises BL1 TNBC or BL2 TNBC.

13. (canceled)

14. The method of claim 1, wherein a tumor characterized by an activated Notch pathway is identified by assessing a) genetic alterations in one or more Notch-regulated genes, b) the gene expression profile or mRNA levels of one or more Notch genes or Notch-regulated genes, c) the levels of one or more Notch proteins or Notch-regulated proteins, or a combination thereof.

15. (canceled)

16. The method of claim 14, wherein said genetic alteration comprises a gain of function (GOF) activating mutation in one or more Notch-related genes, an internal deletion within one or more Notch genes, a functional inactivation of the negative regulatory region (NRR) in one or more Notch-related genes, a functional inactivation of the proline, glutamic acid, serine and threonine rich (PEST) domain in one or more Notch-related genes, a fusion in one or more Notch-related genes, a gene rearrangement in the ectodomain of a Notch gene, or a combination thereof.

17.-20. (canceled)

21. The method of claim 14, wherein said Notch-regulated gene comprises HEY1, NOTCH1, HEYL, NOTCH2, OLFM4, MYC, CDK6, HEY2, KIT, NRARP, MVP, HES6, CDKN2D, NOTCH4, NOTCH3, HES4, HES5, CCND1, HES1, CDKN1B, HES2, or a combination thereof.

22.-23. (canceled)

24. The method of claim 14, wherein the level of cleaved Notch1 protein, or the level of Notch2 protein, Notch3 protein, Notch 4 protein, or combination thereof is assessed.

25.-26. (canceled)

27. The method of claim 1, wherein said composition ern is administered at a dose of 0.3 mg, 0.6 mg, 1.2 mg, 2.4 mg, 4 mg, 6 mg, or 8.4 mg; is administered once a week or once every two weeks; is administered intravenously; or a combination thereof.

28.-29. (canceled)

30. The method of claim 1, further comprising the step of administering a glucocorticoid, a cytotoxic agent, one or more additional therapeutic agents, or a combination thereof.

31.-32. (canceled)

33. The method of claim 30, wherein said glucocorticoid comprises dexamethasone, which is optionally administered prophylactically.

34. (canceled)

35. The method of claim 33, wherein said dexamethasone is administered a) every 6 hours for up to 72 hours, b) at a dose of 4-8 mg, c) orally or intravenously, or d) a combination thereof.

36.-39. (canceled)

40. The method of claim 30, wherein said cytotoxic agent comprises eribulin, vinorelbine, sacituzumab govitecan, or a combination thereof.

41. (canceled)

42. The method of claim 1, wherein said tumor growth comprises tumor outgrowth after treatment withdrawal.

43. The method of claim 1, wherein said subject is exposed to two or more treatment cycles and wherein the tumor growth inhibition is in the second treatment cycle for said subject.

44.-46. (canceled)
